(19) United States
(12) Patent Application Publication
(10) Pub. No.: US 2025/0221676 A1
(43) Pub. Date: Jul. 10, 2025

(54) RADIOGRAPHIC IMAGING APPARATUS

(71) Applicant: CANON KABUSHIKI KAISHA, Tokyo (JP)

(72) Inventors: MASATAKA SUZUKI, Kanagawa (JP); TAKAHIRO KOYANAGI, Kanagawa (JP); TAKASHI FUKUSHIMA, Kanagawa (JP); YOSHITAKA OTSUBO, Kanagawa (JP); TAKAHIRO YAMAMOTO, Kanagawa (JP); SATOMI KYOGOKU, Kanagawa (JP); YURI YOSHIMURA, Tokyo (JP); KAITO UEHATA, Kanagawa (JP)

(21) Appl. No.: 19/093,096

(22) Filed: Mar. 27, 2025

Related U.S. Application Data
(63) Continuation of application No. PCT/JP2023/035049, filed on Sep. 27, 2023.

(30) Foreign Application Priority Data
Sep. 29, 2022 (JP) 2022-156674
Sep. 29, 2022 (JP) 2022-156675
Nov. 1, 2022 (JP) 2022-175708
Nov. 2, 2022 (JP) 2022-176219
Jun. 30, 2023 (JP) 2023-108651
Aug. 3, 2023 (JP) 2023-127129
Sep. 15, 2023 (JP) 2023-149789

(51) Int. Cl. A61B 6/42; G01T 1/20

(52) U.S. Cl. CPC A61B 6/4233; G01T 1/2018

(57) ABSTRACT

A radiographic imaging apparatus includes a radiation detection panel that includes an effective imaging area configured to detect radiation transmitted through a subject and incident on an incident surface, and a housing that accommodates the radiation detection panel. The housing includes a thick section that is thick in a direction normal to the incident surface and located at one end of the housing, and a thin section that is thinner than the thick section and overlaps the effective imaging area at least in part as seen in the direction normal to the incident surface. The thick section includes a grip portion of recessed shape.

### CROSS-REFERENCE TO RELATED APPLICATIONS

This application is a Continuation of International Patent Application No. PCT/JP2023/035049, filed Sep. 27, 2023, which claims the benefit of Japanese Patent Applications No. 2022-175708, filed Nov. 1, 2022, No. 2022-176219, filed Nov. 2, 2022, No. 2022-156674, filed Sep. 29, 2022, No. 2022-156675, filed Sep. 29, 2022, No. 2023-108651, filed Jun. 30, 2023, No. 2023-127129, filed Aug. 3, 2023, No. 2023-149789, filed Sep. 15, 2023, and International Patent Application No. PCT/JP2023/035049, filed Sep. 27, 2023 all of which are hereby incorporated by reference herein in their entirety.

### BACKGROUND OF THE INVENTION

### Field of the Invention

The present invention relates to a radiographic imaging apparatus.

### Background Art

Radiographic imaging apparatuses that detect the intensity distribution of radiation transmitted through an object to obtain a radiographic image are widely used in the field of medical diagnosis. For such radiographic imaging apparatuses, thin and easy-to-handle imaging apparatuses are demanded to enable quick and wide-range imaging of body parts.

To address such a challenge, WO 2020/105706 discusses a radiographic imaging apparatus with a radiation detection unit of reduced thickness. Japanese Patent Application Laid-Open No. 2011-197641 discusses a radiographic imaging apparatus equipped with a grip portion that takes into consideration stability during transportation.

Imaging a subject, such as a patient, using a radiographic imaging apparatus involves an operation where a user, such as a technician, inserts the radiographic imaging apparatus toward the imaging site of the subject, for example. During the insertion operation, careful and accurate operation may be needed, for example, as the subject and the imaging site can contact the imaging apparatus via clothing, cloth, a bag that accommodates the imaging apparatus, etc.

### CITATION LIST

### Patent Literature

PTL 1: WO 2020/105706
PTL 2: Japanese Patent Application Laid-Open No. 2011-197641

The radiographic imaging apparatuses discussed in WO 2020/105706 and Japanese Patent Application Laid-Open No. 2011-197641 are expected to improve handleability by reducing the thickness of the radiation detection unit. However, the radiographic imaging apparatus discussed in WO 2020/105706 can be difficult for the user to adequately grip with fingers, for example. Moreover, simply providing a grip portion as in Japanese Patent Application Laid-Open No. 2011-197641 may not enable the user to maintain adequate grip, for example, when inserting or removing the apparatus into/from a gap between the subject and the contact surface on which the subject lies in a supine position.

### SUMMARY OF THE INVENTION

The present invention has been achieved in view of the foregoing circumstances, and it is an object thereof to provide a radiographic imaging apparatus that improves the user's workability.

Further features of the present invention will become apparent from the following description of exemplary embodiments with reference to the attached drawings.

### BRIEF DESCRIPTION OF THE DRAWINGS

FIG. 1A is a diagram illustrating an example of the appearance of a radiographic imaging apparatus according to a first exemplary embodiment.

FIG. 1B is a diagram illustrating the example of the appearance of the radiographic imaging apparatus according to the first exemplary embodiment.

FIG. 2 is a sectional view illustrating a configuration of the radiographic imaging apparatus according to the first exemplary embodiment.

FIG. 3A is a partial sectional view of a thick section of the radiographic imaging apparatus according to the first exemplary embodiment.

FIG. 3B is a plan view of the thick section of the radiographic imaging apparatus according to the first exemplary embodiment.

FIG. 4A is a diagram illustrating an example of the appearance of a radiographic imaging apparatus according to a second exemplary embodiment.

FIG. 4B is a diagram illustrating the example of the appearance of the radiographic imaging apparatus according to the second exemplary embodiment.

FIG. 5 is a sectional view of grip portions of the radiographic imaging apparatus according to the second exemplary embodiment.

FIG. 6 is a partial sectional view of a thick section of the radiographic imaging apparatus according to the second exemplary embodiment.

FIG. 7A is a plan view of a grip portion of the radiographic imaging apparatus according to the second exemplary embodiment.

FIG. 7B is a plan view of a grip portion of the radiographic imaging apparatus according to the second exemplary embodiment.

FIG. 8A is a diagram illustrating an example of the appearance of a radiographic imaging apparatus according to a third exemplary embodiment.

FIG. 8B is a diagram illustrating the example of the appearance of the radiographic imaging apparatus according to the third exemplary embodiment.

FIG. 9 is a perspective view illustrating a configuration of a grip portion of the radiographic imaging apparatus according to the third exemplary embodiment.

FIG. 10 is a sectional view of the grip portion of the radiographic imaging apparatus according to the third exemplary embodiment.

FIG. 11 is a diagram illustrating an example of the appearance of a radiographic imaging apparatus according to a fourth exemplary embodiment.

FIG. 12A is a diagram illustrating a sectional view of a configuration of the radiographic imaging apparatus according to the fourth exemplary embodiment.

FIG. 12B is a diagram illustrating an enlarged view of the configuration of the radiographic imaging apparatus according to the fourth exemplary embodiment.

FIG. 13 is a diagram illustrating a configuration example of a thin end portion of the radiographic imaging apparatus according to the fourth exemplary embodiment.

FIG. 14A is a diagram illustrating a configuration example of the thin end portion of the radiographic imaging apparatus according to the fourth exemplary embodiment.

FIG. 14B is a diagram illustrating a configuration example of the thin end portion of the radiographic imaging apparatus according to the fourth exemplary embodiment.

FIG. 15A is a diagram illustrating another example of the appearance of the radiographic imaging apparatus according to the fourth exemplary embodiment.

FIG. 15B is a diagram illustrating another example of the appearance of the radiographic imaging apparatus according to the fourth exemplary embodiment.

FIG. 16A is a diagram illustrating another example of the appearance of the radiographic imaging apparatus according to the fourth exemplary embodiment.

FIG. 16B is a diagram illustrating another example of the appearance of the radiographic imaging apparatus according to the fourth exemplary embodiment.

FIG. 17 is a diagram illustrating an example of the appearance of a radiographic imaging apparatus according to a fifth exemplary embodiment.

FIG. 18A is a diagram illustrating a sectional view of a configuration of the radiographic imaging apparatus according to the fifth exemplary embodiment.

FIG. 18B is a diagram illustrating an enlarged view of the configuration of the radiographic imaging apparatus according to the fifth exemplary embodiment.

FIG. 19 is a diagram illustrating a configuration example of a thin end portion of the radiographic imaging apparatus according to the fifth exemplary embodiment.

FIG. 20A is a diagram illustrating a configuration example of the thin end portion of the radiographic imaging apparatus according to the fifth exemplary embodiment.

FIG. 20B is a diagram illustrating a configuration example of the thin end portion of the radiographic imaging apparatus according to the fifth exemplary embodiment.

FIG. 21 is a diagram illustrating an example of the appearance of a radiographic imaging apparatus according to a sixth exemplary embodiment.

FIG. 22A is a diagram illustrating a sectional view of a configuration of the radiographic imaging apparatus according to the sixth exemplary embodiment.

FIG. 22B is a diagram illustrating an enlarged view of the configuration of the radiographic imaging apparatus according to the sixth exemplary embodiment.

FIG. 23 is a diagram illustrating a configuration example of a thin end portion of the radiographic imaging apparatus according to the sixth exemplary embodiment.

FIG. 24 is a diagram illustrating an example of the appearance of a radiographic imaging apparatus according to a seventh exemplary embodiment.

FIG. 25 is a sectional view illustrating a configuration of the radiographic imaging apparatus according to the seventh exemplary embodiment.

FIG. 26 is a plan view illustrating the configuration of the radiographic imaging apparatus according to the seventh exemplary embodiment.

FIG. 27 is a diagram illustrating modification 1 of a corner portion of the radiographic imaging apparatus according to the seventh exemplary embodiment.

FIG. 28A is a diagram illustrating modification 2 of a housing of the radiographic imaging apparatus according to the seventh exemplary embodiment.

FIG. 28B is a diagram illustrating modification 2 of the housing of the radiographic imaging apparatus according to the seventh exemplary embodiment.

FIG. 29A is a diagram illustrating an example of the appearance of a radiographic imaging apparatus according to an eighth exemplary embodiment.

FIG. 29B is a diagram illustrating the example of the appearance of the radiographic imaging apparatus according to the eighth exemplary embodiment.

FIG. 30 is a diagram illustrating modification 1 of a groove of the radiographic imaging apparatus according to the eighth exemplary embodiment.

FIG. 31A is a diagram illustrating an example of the appearance of a radiographic imaging apparatus according to a ninth exemplary embodiment.

FIG. 31B is a diagram illustrating the example of the appearance of the radiographic imaging apparatus according to the ninth exemplary embodiment.

FIG. 32 is a diagram illustrating modification 1 of a protrusion of the radiographic imaging apparatus according to the ninth exemplary embodiment.

FIG. 33A is a diagram illustrating an example of the appearance of a radiographic imaging apparatus according to a tenth exemplary embodiment.

FIG. 33B is a diagram illustrating the example of the appearance of the radiographic imaging apparatus according to the tenth exemplary embodiment.

FIG. 34 is a sectional view illustrating a configuration of the radiographic imaging apparatus according to the tenth exemplary embodiment.

FIG. 35 is a sectional view illustrating the configuration of the radiographic imaging apparatus according to the tenth exemplary embodiment.

FIG. 36 is a diagram illustrating a modification of the radiographic imaging apparatus according to the tenth exemplary embodiment.

FIG. 37A is a diagram illustrating an example of the appearance of a radiographic imaging apparatus according to an eleventh exemplary embodiment.

FIG. 37B is a diagram illustrating the example of the appearance of the radiographic imaging apparatus according to the eleventh exemplary embodiment.

FIG. 38A is a diagram illustrating an example of the appearance of a radiographic imaging apparatus according to a twelfth exemplary embodiment.

FIG. 38B is a diagram illustrating the example of the appearance of the radiographic imaging apparatus according to the twelfth exemplary embodiment.

FIG. 38C is a diagram illustrating the example of the appearance of the radiographic imaging apparatus according to the twelfth exemplary embodiment.

FIG. 39 is a sectional view illustrating a configuration of the radiographic imaging apparatus according to the twelfth exemplary embodiment.

FIG. 40A is a diagram illustrating modification 1 of the configuration of the radiographic imaging apparatus according to the twelfth exemplary embodiment.

FIG. 40B is a diagram illustrating modification 1 of the configuration of the radiographic imaging apparatus according to the twelfth exemplary embodiment.

FIG. 40C is a diagram illustrating modification 1 of the configuration of the radiographic imaging apparatus according to the twelfth exemplary embodiment.

FIG. 41 is a diagram illustrating modification 2 of the configuration of the radiographic imaging apparatus according to the twelfth exemplary embodiment.

FIG. 42A is a diagram illustrating an example of the appearance of a radiographic imaging apparatus according to a thirteenth exemplary embodiment.

FIG. 42B is a diagram illustrating the example of the appearance of the radiographic imaging apparatus according to the thirteenth exemplary embodiment.

FIG. 43 is a diagram illustrating modification 1 of a configuration of the radiographic imaging apparatus according to the thirteenth exemplary embodiment.

FIG. 44A is a diagram illustrating an example of the appearance of a radiographic imaging apparatus according to a fourteenth exemplary embodiment.

FIG. 44B is a diagram illustrating the example of the appearance of the radiographic imaging apparatus according to the fourteenth exemplary embodiment.

FIG. 45A is a diagram illustrating an example of the appearance of a radiographic imaging apparatus according to a fifteenth exemplary embodiment.

FIG. 45B is a diagram illustrating the example of the appearance of the radiographic imaging apparatus according to the fifteenth exemplary embodiment.

FIG. 45C is a diagram illustrating the example of the appearance of the radiographic imaging apparatus according to the fifteenth exemplary embodiment.

FIG. 45D is a diagram illustrating the example of the appearance of the radiographic imaging apparatus according to the fifteenth exemplary embodiment.

FIG. 46A is a diagram illustrating modification 1 of a configuration of the radiographic imaging apparatus according to the fifteenth exemplary embodiment.

FIG. 46B is a diagram illustrating modification 1 of the configuration of the radiographic imaging apparatus according to the fifteenth exemplary embodiment.

FIG. 46C is a diagram illustrating modification 1 of the configuration of the radiographic imaging apparatus according to the fifteenth exemplary embodiment.

FIG. 46D is a diagram illustrating modification 1 of the configuration of the radiographic imaging apparatus according to the fifteenth exemplary embodiment.

FIG. 47 is a diagram illustrating an example of the appearance of a radiographic imaging apparatus according to a sixteenth exemplary embodiment.

FIG. 48 is a diagram illustrating an example of a schematic configuration of a radiographic imaging system according to a seventeenth exemplary embodiment.

FIG. 49 is a view of a radiographic imaging apparatus according to the seventeenth exemplary embodiment seen from a rear side.

FIG. 50A is a diagram illustrating an example of an internal configuration of the radiographic imaging apparatus according to the seventeenth exemplary embodiment as seen from the rear side.

FIG. 50B is a diagram illustrating the example of the internal configuration of the radiographic imaging apparatus according to the seventeenth exemplary embodiment as seen from the rear side.

FIG. 51 is a sectional view illustrating a configuration of the radiographic imaging apparatus according to the seventeenth exemplary embodiment.

FIG. 52A is a diagram illustrating an example of an internal configuration of a radiographic imaging apparatus according to an eighteenth exemplary embodiment as seen from a rear side.

FIG. 52B is a diagram illustrating the example of the internal configuration of the radiographic imaging apparatus according to the eighteenth exemplary embodiment as seen from the rear side.

FIG. 53 is a sectional view illustrating a configuration of the radiographic imaging apparatus according to the eighteenth exemplary embodiment.

FIG. 54 is a diagram illustrating an example of a schematic configuration of a radiographic imaging system according to a nineteenth exemplary embodiment.

FIG. 55 is a sectional view illustrating a configuration of a radiographic imaging apparatus according to the nineteenth exemplary embodiment.

### DESCRIPTION OF THE EMBODIMENTS

Exemplary embodiments of the present invention will be specifically described with reference to the attached drawings. It should be noted that dimensions and detailed structures described in the exemplary embodiments of the present invention are not limited to those described in the description or illustrated in the drawings. As employed herein, radiations shall include not only X-rays but x-rays, β-rays, γ-rays, particle beams, and cosmic rays as well.

### First Exemplary Embodiment

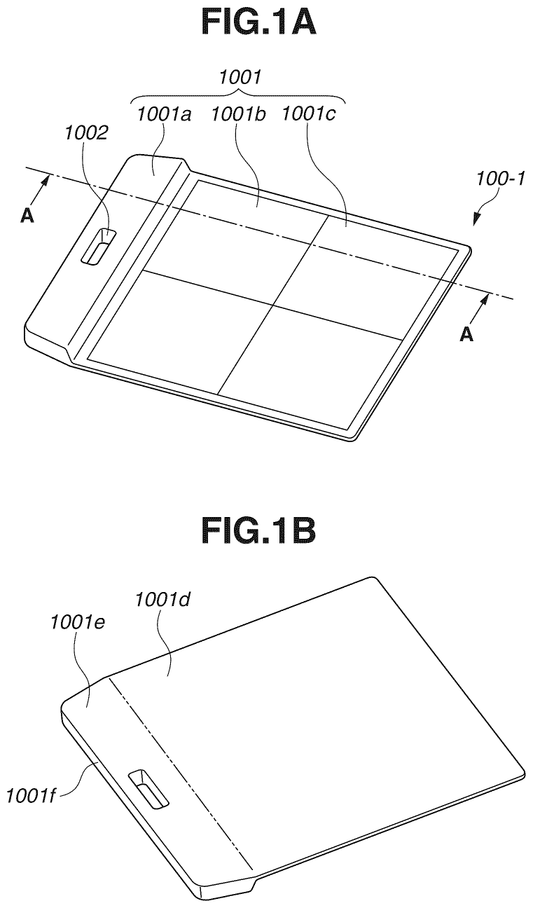

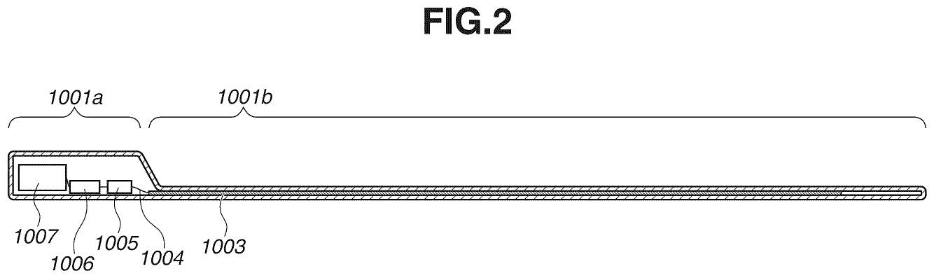

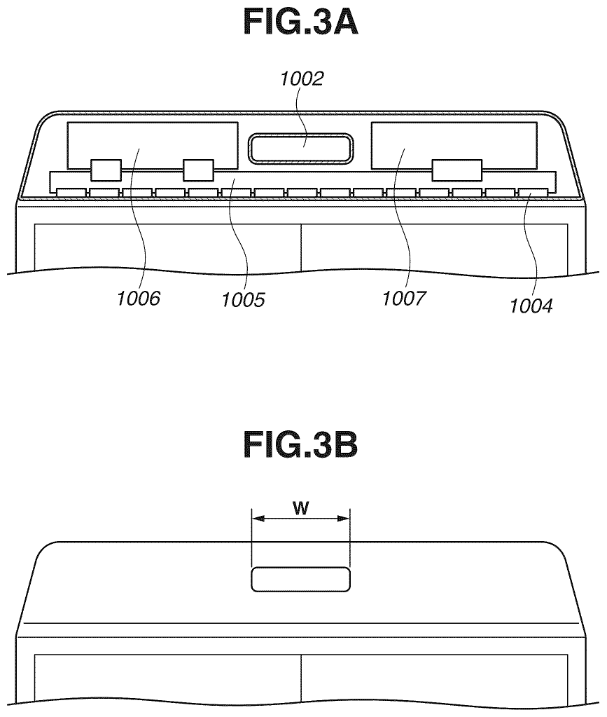

FIGS. 1A and 1B are diagrams illustrating an example of the appearance of a radiographic imaging apparatus 100-1 according to a first exemplary embodiment. Specifically, FIG. 1A is an external perspective view of the radiographic imaging apparatus 100-1 seen in an incident direction of radiation. FIG. 1B is an external perspective view as seen from a side opposite to the incident direction. FIG. 2 is a sectional view of the radiographic imaging apparatus 100-1 taken along line A-A illustrated in FIG. 1A, seen in the direction of the arrows. FIG. 3A is a partially sectional plan view of a thick section seen in the incident direction of the radiation. FIG. 3B is a partial plan view of the thick section seen in the incident direction of the radiation.

The radiographic imaging apparatus 100-1 detects radiation emitted from a not-illustrated radiation generation apparatus and transmitted through a subject, using a radiation detection panel 1003. A radiographic image obtained by the radiographic imaging apparatus 100-1 is transferred to outside, displayed on a monitor or the like, and used for diagnosis etc.

The interior of the radiographic imaging apparatus 100-1 is covered by a housing 1001 including a thick section 1001a and a thin section 1001b.

The radiation detection panel 1003 includes a phosphor layer that converts the amount of radiation into light and an imaging detection panel that detects light as an electric charge.

The imaging detection panel includes a plurality of pixel devices two-dimensionally arranged on an insulating substrate, each including a conversion element for converting the amount of radiation into the amount of charge and a switch element for transferring an electrical signal based on the charge. The insulating substrate is suitably formed of glass, a flexible plastic, or the like, for example. For the phosphor layer, materials such as CsI (cesium iodine) are suitably used. A phosphor protective film for protecting the phosphor from moisture may also be provided.

The radiation detection panel 1003 is connected to a reading circuit 1005, a control substrate 1006, and the like via flexible circuit boards 1004. The reading circuit 1005 reads the electrical signals from the pixel devices of the radiation detection panel 1003. The control substrate 1006 performs electrical signal control, direct-current voltage conversion, and the like of driving circuits and the like for supplying driving signals having a voltage to make the switch elements conductive to the switch elements.

In the foregoing description, the radiation detection panel 1003 is described to be of so-called indirect conversion type, including the phosphor layer and the pixel devices. However, this is not restrictive. For example, the radiation detection panel 1003 may be of so-called direct conversion type, including a conversion element unit where conversion elements formed of a-Se or the like and electrical elements such as thin-film transistors (TFTs) are two-dimensionally arranged.

The radiation detection panel 1003 is disposed inside the thin section 1001b. As another component, an impact absorption layer is located between the incident surface side and the radiation detection panel 1003 to protect the radiation detection panel 1003 from external impact and the like. The impact absorption layer is suitably formed of a foamed resin, gel, etc., whereas other materials may also be used.

To achieve portability and strength in a compatible manner, the housing 1001 is suitably formed of a magnesium alloy, fiber-reinforced plastic, plastic, or the like, whereas other materials may also be used. In particular, an effective imaging area surface 1001c of the radiation detection panel is suitably formed of a carbon fiber-reinforced plastic or the like with high radiation transmittance and excellent lightweight properties, whereas other materials may also be used. A thin section rear surface 1001d is suitably formed of a radiation-shielding material containing one of heavy metals Pb, Ba, Ta, Mo, and W, or stainless steel, for example, whereas other materials may also be used.

When imaging a subject, such as a patient, the radiographic imaging apparatus can be placed immediately behind the imaging site of the subject. In doing so, due to a step created by the thickness of the radiographic imaging apparatus, the subject and the edge of the radiographic imaging apparatus come into contact to cause a reaction force, and the subject may feel discomfort.

Conventionally, radiographic imaging apparatuses have often been provided in sizes compliant with International Organization for Standardization (ISO) 4090:2001, often with a thickness of approximately 15 mm to 16 mm. In the present exemplary embodiment, the thin section 1001b has a thickness of 8.0 mm. This reduces the step created by the thickness of the radiographic imaging apparatus 100-1 and can ease the reaction force occurring between the subject and the end portion of the radiographic imaging apparatus 100-1 during imaging.

To obtain such effects, the thin section 1001b does not need to be limited to the thickness of 8.0 mm and can be thinner. In particular, thicknesses less than 10.0 mm have been confirmed to be effective.

As illustrated in FIGS. 2 and 3A, the thick section 1001a includes the reading circuit 1005, the control substrate 1006, and a secondary battery 1007 (such as a lithium-ion battery, an electric double layer capacitor, and an all-solid-state battery). A wireless module unit for performing transmission and reception with not-illustrated external devices, an external connection terminal unit intended for power supply and data communication from external devices, a user interface unit that implements status control and a display, and the like are also disposed.

To improve user grip, the thick section 1001a is provided with a grip portion 1002. As illustrated in FIGS. 2 and 3B, the grip portion 1002 has the shape of a hole that runs through to the rear side of the thick section 1001a. The grip portion 1002 desirably has a width W of 60 mm or more, which allows for two to three fingers to be placed in it, assuming that the distal interphalangeal joint of a finger is approximately 20 mm in width.

The provision of the foregoing grip portion 1002 in the thick section 1001a can improve the grippability and portability of the radiographic imaging apparatus 100-1. This facilitates the user to handle the radiographic imaging apparatus 100-1 when performing operations such as insertion and removal into/from directly under a subject in a supine position, and enables quick imaging.

For easier placement of fingers when the rear surface of the radiographic imaging apparatus 100-1 is in contact with the floor surface, it is even more suitable to provide a gap between a thick section side surface 1001f and the floor surface. For example, to provide the gap, a thick section rear surface 1001e may be inclined relative to the thin section rear surface 1001d, instead of configuring the thick section rear surface 1001e and the thin section rear surface 1001d on the same plane.

### Second Exemplary Embodiment

Next, a grip portion of a radiographic imaging apparatus according to a second exemplary embodiment will be described. A description of configurations similar to those of the first exemplary embodiment will be omitted as appropriate.

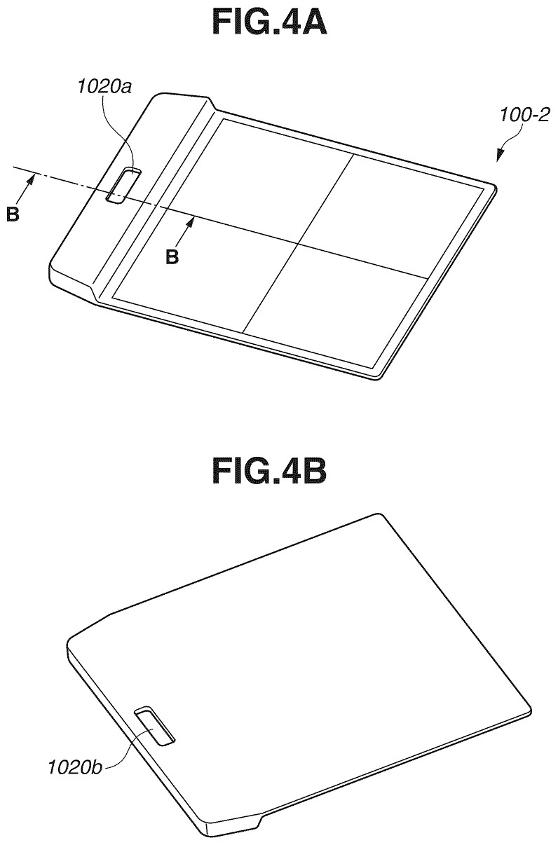

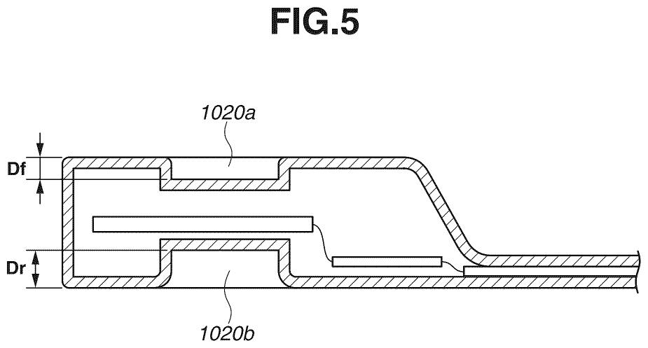

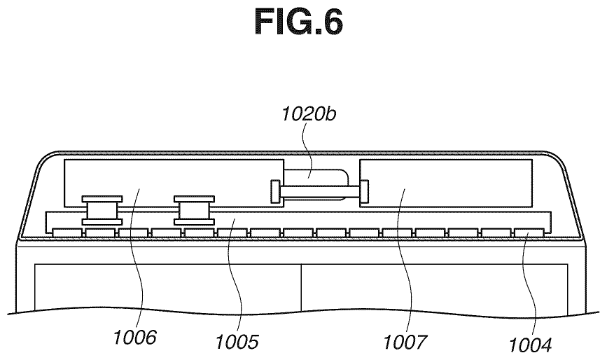

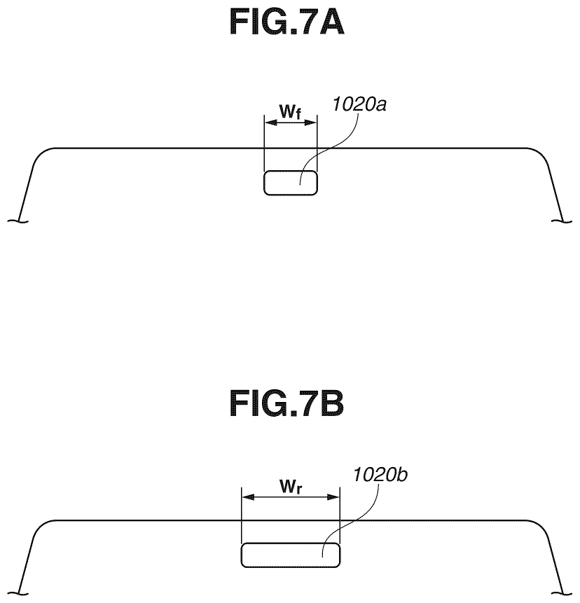

FIGS. 4A and 4B are diagrams illustrating an example of the appearance of a radiographic imaging apparatus 100-2 according to the second exemplary embodiment. Specifically, FIG. 4A is an external perspective view of the radiographic imaging apparatus 100-2 seen in the incident direction of radiation. FIG. 4B is an external perspective view as seen from the side opposite to the incident direction. FIG. 5 is a sectional view of grip portions 1020a and 1020b taken along line B-B illustrated in FIG. 4A, seen in the direction of the arrows. FIG. 6 is a partial sectional view of a thick section 1001a seen in the incident direction of the radiation. FIG. 7A is a partial plan view of the thick section 1001a seen in the incident direction of the radiation. FIG. 7B is a partial plan view of the thick section 1001a seen from the side opposite to the incident direction.

The radiographic imaging apparatus 100-2 illustrated in FIGS. 4A and 4B includes a grip portion 1020a of recessed shape in the incident surface side of the thick section 1001a, and a grip portion 1020b of recessed shape in the rear side opposed to the incident surface. Since the grip portions 1020a and 1020b have a recessed shape, the grip portions 1020a and 1020b and the structures included in a thick section 1001a, such as a control substrate 1006 and a secondary battery 1007, can be located at positions overlapping in a plan view seen in a direction normal to the incident surface as illustrated in FIG. 6. This can provide more layout space for the control substrate 1006 and the secondary battery 1007 included within compared to the grip portion that is a through hole.

The grip portions 1020a and 1020b can be made deeper by configuring the structures located at positions overlapping with the grip portions 1020a and 1020b in a plan view as thin items, such as a bare board without mounted parts and an FFC.

In gripping the radiographic imaging apparatus 100-2 and inserting or removing the radiographic imaging apparatus 100-2 into/from a gap between a subject and the contact surface where the subject lies in a supine position, the grip portion 1020a is gripped with the thumb (first finger) and the grip portion 1020b with the other fingers. As illustrated in FIGS. 5, 7A, and 7B, a depth Df of the grip portion 1020a and a depth Dr of the grip portion 1020b are therefore desirably Df≤Dr, and a width Wf of the grip portion 1020a and a width Wr of the gription 1020b Wf≤Wr. Moreover, considering the lengths and widths of the distal joints of the fingers, it is desirable that Df+Dr≥5 mm, Wf≥20 mm, and Wr≥60 mm.

### Third Exemplary Embodiment

Next, a grip portion of a radiographic imaging apparatus according to a third exemplary embodiment will be described. A description of configurations similar to those of the first and second exemplary embodiments will be omitted as appropriate.

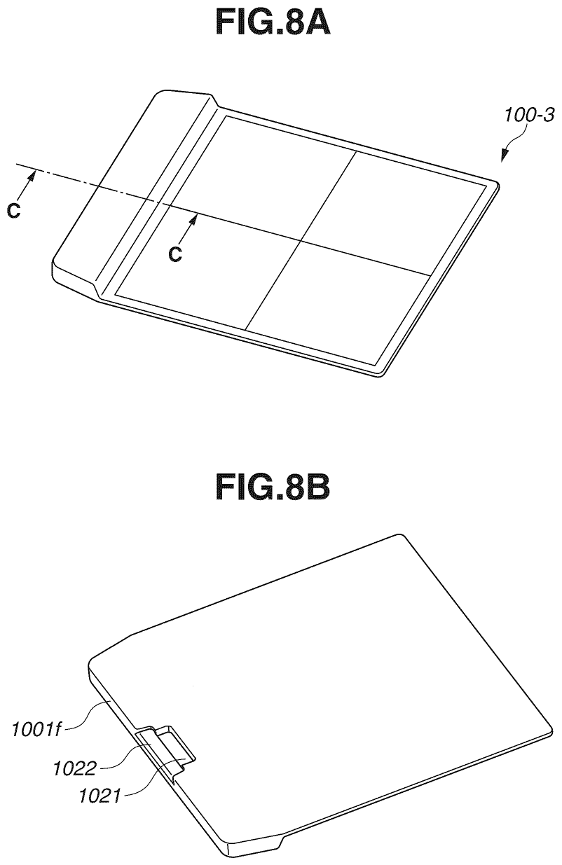

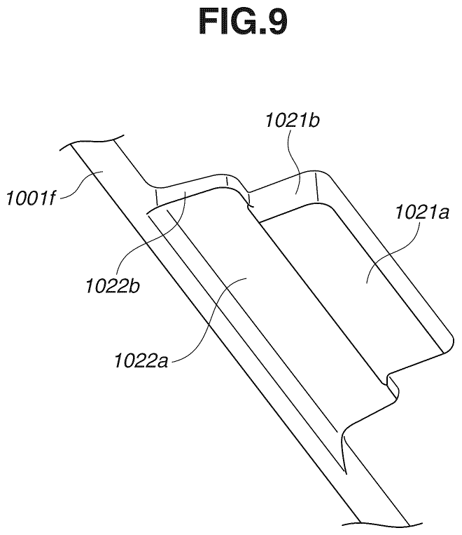

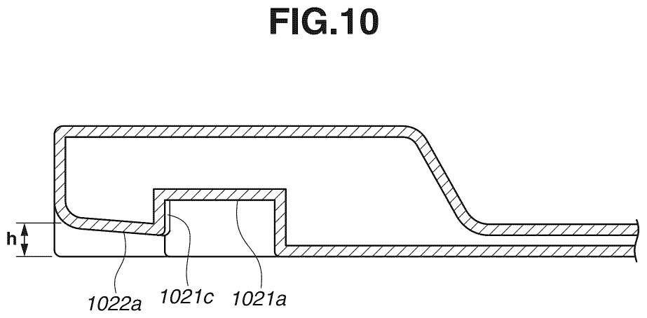

FIGS. 8A and 8B are diagrams illustrating an example of the appearance of a radiographic imaging apparatus 100-3 according to the third exemplary embodiment. Specifically, FIG. 8A is an external perspective view of the radiographic imaging apparatus 100-3 seen in the incident direction of radiation. FIG. 8B is an external perspective view as seen from the side opposite to the incident direction. FIG. 9 is a perspective view of a grip portion 1021 and a hand access portion 1022. FIG. 10 is a sectional view of the grip portion 1021 taken along line C-C illustrated in FIG. 8A, seen in the direction of the arrows.

As illustrated in FIGS. 8A and 8B, the radiographic imaging apparatus 100-3 has the grip portion 1021 disposed in the rear side of a thick section 1001a opposite to the incident direction of the radiation. The hand access portion 1022 is disposed in a thick section side surface 1001f.

As illustrated in FIGS. 9 and 10, the grip portion 1021 includes a bottom surface 1021a, side surfaces 1021b, and a side surface 1021c. The hand access portion 1022 includes a bottom wall 1022a and side walls 1022b. The surface of the bottom wall 1022a is an example of a hand access surface. The surfaces of the side walls 1022b are an example of side walls.

The side walls 1022b according to the present exemplary embodiment are orthogonal to the incident surface of the radiation. The bottom wall 1022a adjoins the thick section side surface 1001f and the side surface 1021c of the grip portion 1021. The side walls 1022b adjoin the bottom wall 1022a, the thick section side surface 1001f, and a thin section rear surface 1001d. The bottom wall 1022a widens from the grip portion 1021 toward the thick section side surface 1001f. Moreover, the bottom wall 1022a is inclined to approach the incident surface from the grip portion 1021 toward the thick section side surface 1001f. To facilitate the insertion of fingertips, the hand access portion 1022 desirably has a height h of h≥5 mm.

The foregoing inclination provides the effect of facilitating finger access to the hand access portion 1022 from the housing side surface and increasing the side wall height of the grip portion 1021 so that fingers can be easily placed in the grip portion 1021.

Since the bottom wall 1022a of the hand access portion 1022 adjoins the side surface 1021c of the grip portion 1021, fingers can be easily placed without visual observation in a continuous action of sliding the fingers from the hand access portion 1022 to the grip portion 1021.

While the preferred first to third exemplary embodiments of the present invention have been described above, these exemplary embodiments are not restrictive, and various modifications and changes can be made without departing from the gist thereof. Moreover, the foregoing exemplary embodiments may be combined as appropriate.

The first to third exemplary embodiments of the present invention include the features described in the following supplementary notes.

### [Supplementary Note 1]

A radiographic imaging apparatus including
a radiation detection panel that includes an effective imaging area configured to detect radiation transmitted through a subject and incident on an incident surface, and
a housing that accommodates the radiation detection panel,
wherein the housing includes a thick section that is thick in a direction normal to the incident surface and located at one end of the housing and a thin section that is thinner than the thick section and overlaps the effective imaging area at least in part as seen in the direction normal to the incident surface, and
wherein the thick section includes a grip portion of recessed shape.

### [Supplementary Note 2]

The radiographic imaging apparatus according to supplementary note 1,
wherein the thick section includes a thick incident surface where the radiation is incident and a thick rear surface opposed to the thick incident surface, and
wherein the grip portion is disposed in at least either of the thick incident surface and the thick rear surface.

### [Supplementary Note 3]

The radiographic imaging apparatus according to supplementary note 2, wherein the grip portion includes an incident-side grip portion that is a grip portion disposed in the thick incident surface and a rear-side grip portion that is a grip portion disposed in the thick rear surface.

### [Supplementary Note 4]

The radiographic imaging apparatus according to supplementary note 3, wherein the incident-side grip portion has a length of 20 mm or more and the rear-side grip portion has a length of 60 mm or more in a direction along a border between the thin section and the thick section.

### [Supplementary Note 5]

The radiographic imaging apparatus according to supplementary note 3 or 4, wherein a depth of the rear-side grip portion from the thick rear surface is greater than a depth of the incident-side grip portion from the thick incident surface.

### [Supplementary Note 6]

The radiographic imaging apparatus according to any one of supplementary notes 3 to 5, wherein a sum of a/the depth of the incident-side grip portion from the thick incident surface and a/the depth of the rear-side grip portion from the thick rear surface is 5 mm or more.

### [Supplementary Note 7]

The radiographic imaging apparatus according to any one of supplementary notes 2 to 6,
wherein the thick section includes a thick side surface connecting the thick incident surface and the thick rear surface, and
wherein a hand access portion of recessed shape is disposed to adjoin the thick side surface and the thick rear surface.

### [Supplementary Note 8]

The radiographic imaging apparatus according to supplementary note 7, wherein the hand access portion includes a hand access surface adjoining the grip portion and the thick side surface.

### [Supplementary Note 9]

The radiographic imaging apparatus according to supplementary note 8, wherein a depth of the hand access surface from the thick rear surface is smaller than that of the grip portion.

### [Supplementary Note 10]

The radiographic imaging apparatus according to any one of supplementary notes 2 to 9, wherein the thick rear surface is inclined with respect to a surface of the thin section opposed to the incident surface.

### [Supplementary Note 11]

The radiographic imaging apparatus according to any one of supplementary notes 1 to 10,
wherein the thick section includes a control unit configured to control the radiation detection panel and a power supply unit configured to supply power to each component of the radiographic imaging apparatus, and
wherein the grip portion is located at a position overlapping at least either of the control unit and the power supply unit as seen in the direction normal to the incident surface.

According to the features described in the foregoing supplementary notes 1 to 11, a radiographic imaging apparatus that has favorable grippability and improved user workability is provided.

### Fourth Exemplary Embodiment

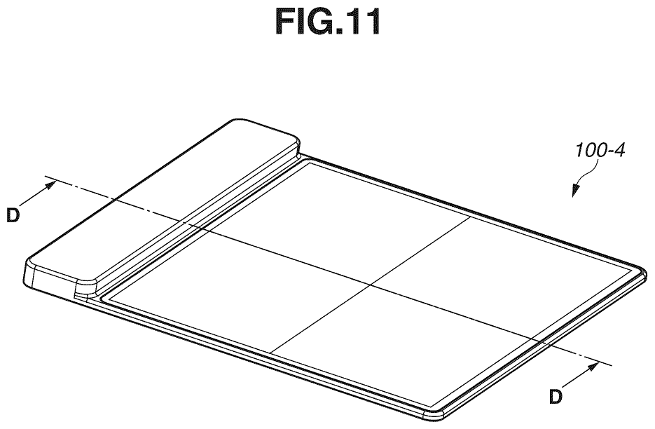

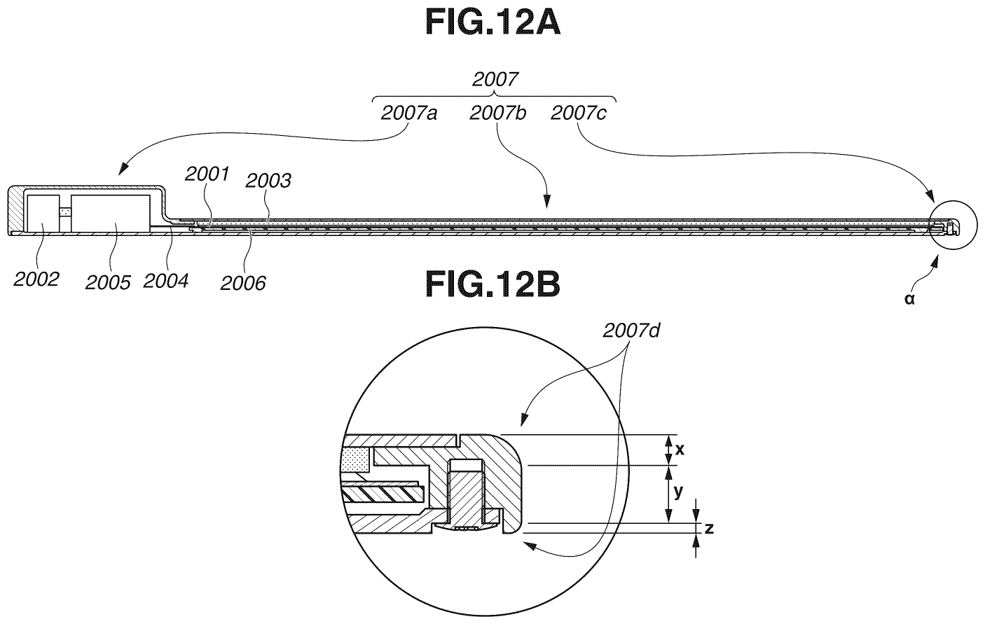

FIG. 11 is a diagram illustrating an example of the appearance of a radiographic imaging apparatus 100-4 according to a fourth exemplary embodiment. Specifically, FIG. 11 illustrates the appearance of the radiographic imaging apparatus 100-4 with a radiation detection panel 2001 built in. FIG. 12A illustrates a sectional view of the radiographic imaging apparatus 100-4 taken along line D-D illustrated in FIG. 11. FIG. 12B illustrates an enlarged view of part α in FIG. 12A.

The radiographic imaging apparatus 100-4 detects radiation emitted from a not-illustrated radiation generation apparatus and transmitted through a subject, using the radiation detection panel 2001. A radiographic image obtained by the radiographic imaging apparatus 100-4 is transferred to outside, displayed on a monitor or the like, and used for diagnosis etc.

The radiation detection panel 2001 according to the present exemplary embodiment is of an indirect conversion system, including a sensor substrate on which a large number of photoelectric conversion elements (sensors) are arranged, a phosphor layer (scintillator layer) that is located on the sensor substrate, and a phosphor protective film. However, this is not restrictive. For example, the radiation detection panel 2001 may be of so-called direct conversion type, including a conversion element unit where conversion elements formed of a-Se or the like and electrical elements such as TFTs are two-dimensionally arranged.

The radiation detection panel 2001 includes some or all of the photoelectric conversion elements (sensors) in its effective imaging area. The effective imaging area is an area that is capable of radiographic imaging and where the radiographic image is actually generated. In the present exemplary embodiment, the effective imaging area has a substantially rectangular shape as seen in a direction normal to an incident surface of the radiographic imaging apparatus 100-4 where the radiation is incident. However, the shape is not limited thereto, and may be substantially polygonal.

The phosphor protective film is formed of a material with low moisture permeability, and is provided to protect the phosphor from deliquescence due to moisture.

The radiation detection panel 2001 is connected to a flexible circuit board 2004. A control substrate 2005 that reads detection signals from the radiation detection panel 2001 and processes the read detection signals is further connected to the flexible circuit board 2004. The radiographic imaging apparatus 100-4 includes a housing (exterior casing) 2007 that accommodates the radiation detection panel 2001. The sensor substrate of the radiation detection panel 2001 is suitably formed of, but not limited to, materials such as glass and flexible plastic.

The housing 2007 includes a thick section 2007a that is located at one end of the housing 2007 and thick in the direction normal to the incident surface, and a thin section 2007b that is thinner than the thick section 2007a. The effective imaging area of the radiation detection panel 2001 is located in the thin section 2007b as seen in the direction normal to the incident surface. A thin end portion 2007c that is an end portion of the thin section 2007b as seen in the direction normal to the incident surface includes sloped portions 2007d. In other words, the thin section 2007b includes the sloped portions 2007d at a side other than those adjoining the thick section 2007a.

The thick section 2007a includes at least a part of the control substrate 2005. The thick section 2007a also includes a battery 2002 for supplying necessary power to the components of the radiographic imaging apparatus 100-4. Examples of the battery 2002 include a lithium-ion battery, an electric double layer capacitor, and an all-solid-state battery, whereas other batteries may be used. The thin section 2007b can be made thin by accommodating relatively thick components in the thick section 2007a. The thin section 2007b may be configured as a flexible component, in which case rigid components such as the battery 2002 are suitably accommodated in the thick section 2007a.

To achieve portability and strength in a compatible manner, the housing 2007 is suitably formed of a magnesium alloy, aluminum alloy, fiber-reinforced plastic, plastic, or the like, whereas other materials may also be used. In particular, the incident surface of the thin section 2007b where the effective imaging area is located and the radiation is incident is suitably formed of a material with high radiation transmittance and excellent lightweight properties. For example, a carbon fiber-reinforced plastic or the like is used, whereas other materials may also be used.

A cushioning material 2003 is disposed between the radiation detection panel 2001 and the incident surface of the housing 2007, whereby the radiation detection panel 2001 is protected from external force and the like. The cushioning material 2003 can be formed of a foamed resin, gel, etc., whereas other materials may also be used. A support base 2006 is provided to support the radiation detection panel 2001. The support base 2006 is suitably formed of a material with excellent lightweight properties. Examples include magnesium alloys, aluminum alloys, fiber-reinforced plastic, and plastic, whereas other materials may also be used.

Next, the shape of the housing 2007 according to the present exemplary embodiment will be described. Conventionally, radiographic imaging apparatuses have often been provided in sizes compliant with International Organization for Standardization (ISO) 4090:2001. Many radiographic imaging apparatuses are therefore formed with a thickness of approximately 15 mm to 16 mm.

When imaging a subject using a radiographic imaging apparatus, the radiographic imaging apparatus can be placed immediately behind the imaging site of the subject. In doing so, due to a step created by the thickness of the radiographic imaging apparatus, the end portion of the radiographic imaging apparatus comes into contact with the subject to cause a reaction force, and the subject may feel discomfort. Moreover, a large insertion force may be needed, which can hinder quick imaging operation.

In the present exemplary embodiment, the thin section 2007b has a housing thickness of 8.0 mm. This reduces the step created by the thickness of the radiographic imaging apparatus 100-4 and can ease the reaction force occurring between the subject and the edge of the radiographic imaging apparatus 100-4 during imaging. In particular, to obtain such effects, the housing thickness of the thin section 2007b of less than 10.0 mm has been confirmed to be effective.

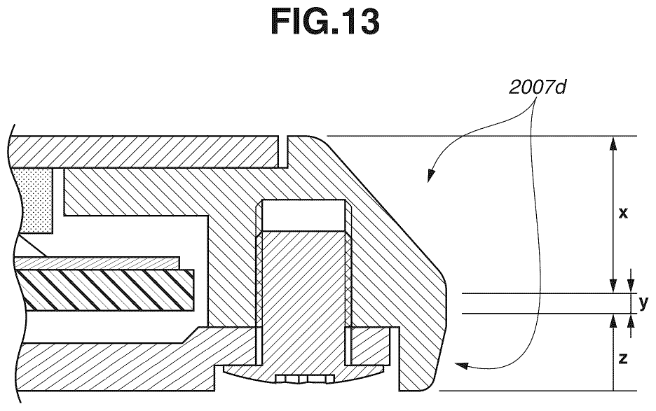

In the present exemplary embodiment, not only is the radiographic imaging apparatus 100-4 provided with the thin section 2007b, but also the sloped portions 2007d are provided on the edge of the thin end portion 2007c that serves as the insertion leading edge during insertion among the plurality of edges of the thin section 2007b. The provision of the sloped portions 2007d can configure the end portion of the radiographic imaging apparatus 100-4 small in thickness. The sloped portions 2007d can be disposed on both the incident surface side of the housing 2007 and the surface (bottom surface) opposed to the incident surface. The sloped portions 2007d are not limited to the round-cornered shape of FIG. 12B, and may have a chamfered shape as illustrated in FIG. 13.

As illustrated in FIGS. 12B and 13, the heights of the sloped portions 2007d and the side surface of the housing 2007 in the radiation incident direction are referred to as height x, height y, and height z. As illustrated in FIG. 13, height x is desirably larger compared to heights y and z. By contrast, height y is desirably the smallest. Height x is desirably more than or equal to one half of the thickness of the thin section 2007b. The side surface constituting height y is desirably located closer to the bottom side. Such a configuration improves the ease of insertion of the radiographic imaging apparatus 100-4.

The foregoing configuration can increase the contact area between the radiographic imaging apparatus 100-4 and the subject during the insertion operation of the radiographic imaging apparatus 100-4. Moreover, reaction force can be caused in directions different from the insertion direction as well. This reduces the pressure that the subject such as a patient feels from the reaction force, and is expected to reduce the discomfort of the subject such as a patient. Furthermore, the provision of the sloped portion 2007d on the bottom side as well is expected to provide the effect of improving workability since the radiographic imaging apparatus 100-4 is less likely to be caught on bed sheets and the like during insertion.

The highly portable radiographic imaging apparatus 100-4 can be subjected to impact and the like, such as when inadvertently dropped. To ensure normal operation of the radiation detection function even on such occasions, the radiographic imaging apparatus 100-4 is desired to be designed in consideration of impact resistance. Strength against static pressure is also needed, since imaging may be performed with the weight of the subject such as a patient directly applied during imaging.

Radiographic imaging apparatuses with thin sections may have low strength against external force because of the thin external shape, compared to radiographic imaging apparatuses of thicker configuration. Being low-profile also limits components that can be incorporated inside. Since thick components are hard to incorporate, it is challenging to improve rigidity, and appropriate bending rigidity and strength are difficult to obtain.

In the present exemplary embodiment, the housing 2007 includes three parts, namely, an incident surface unit, a bottom surface unit, and a lateral side unit. The lateral side unit that is thick in the direction normal to the incident surface is sandwiched between the incident surface unit and the bottom surface unit. The incident surface unit and the bottom surface unit are planarly bonded using an adhesive, a pressure-sensitive adhesive, or the like. The adhesion can prevent a decrease in the rigidity of the fastening portions. Although not illustrated in the drawings, the incident surface unit and the side surface unit may be integrated for improved rigidity.

Meanwhile, the lateral unit can be formed of a material with lower bending modulus and lower specific gravity than those of the incident surface unit and the bottom surface unit for the sake of weight reduction. A material with high bending modulus can be disposed on the surface layer side of the thin end portion 2007c to improve the bending strength of the entire housing 2007.

The lateral side unit and the bottom surface unit are fastened by screws. When a large bending or twisting occurs on the thin section 2007b, the external force can thus be expected to be absorbed through effects such as sliding of the screw seats and the point fastening.

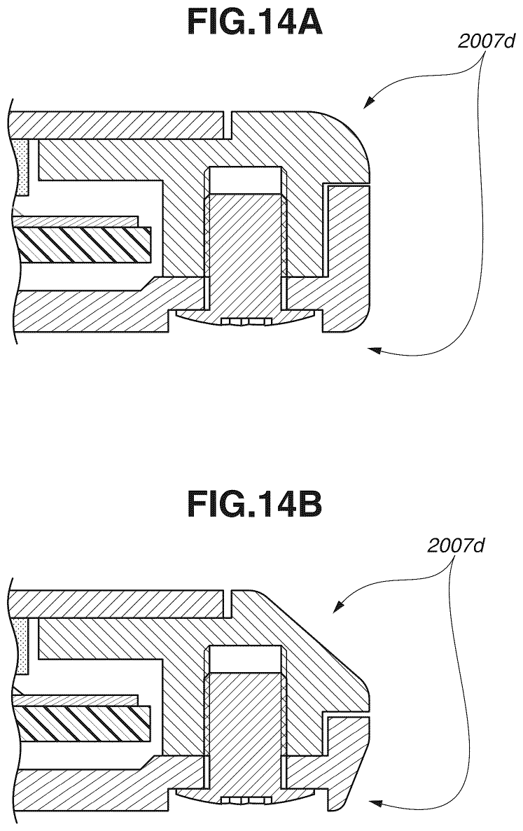

Furthermore, as illustrated in FIGS. 14A and 14B, a vertical wall may be erected so that the bottom surface unit constitutes a part of the lateral side for improved rigidity. The lateral side unit or the incident surface unit may be integrated with a part of the thick section 2007a for improved rigidity.

The provision of the sloped portions 2007d is also expected to ease stress when the housing 2007 is distorted. As illustrated in FIG. 14B, the sloped portions 2007d are desirably located to overlap the fastening portions as seen in the direction normal to the incident surface, so that a larger area can be covered. Although not illustrated, a rubber or other waterproof gasket can be interposed to make the housing 2007 waterproof.

As seen in the direction normal to the incident surface, the thick section 2007a is desirably located at the end opposite to the thin end portion 2007c where the sloped portions 2007d are disposed. When applying force in the insertion direction, the user can apply the force to the wide surface of the thick section 2007a. This is expected to reduce the pressure on the user due to the contact force and improve workability. Like the sloped portions 2007d of the thin end portion 2007c, the thick section 2007a may also be provided with sloped portions at locations where the incident surface adjoins the side surfaces. This is expected to further reduce the pressure.

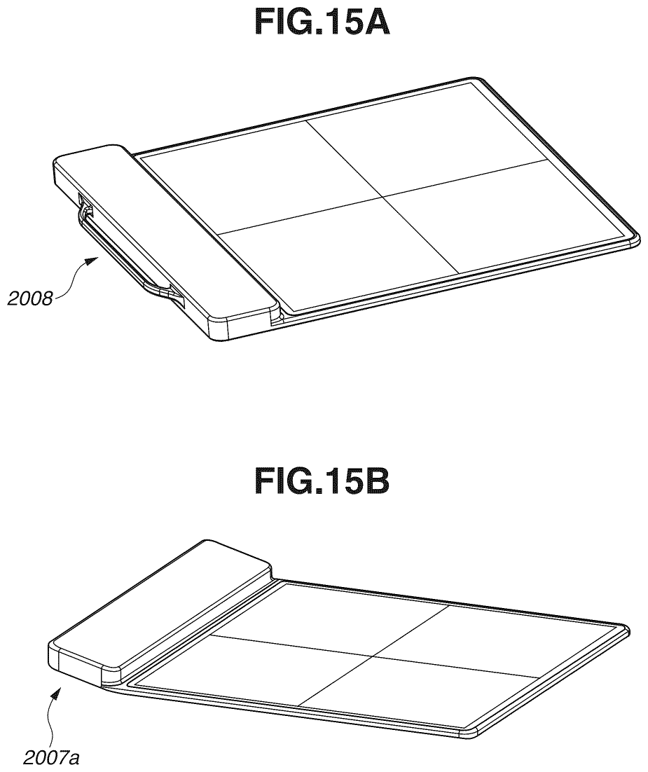

As illustrated in FIG. 15A, in view of portability and workability during insertion and removal, a grip portion 2008 may be disposed at the end of the thick section 2007a opposite to the side of the thin end portion 2007c where the sloped portions 2007d are disposed. To facilitate user grip during insertion, as illustrated in FIG. 15B, the thick section 2007a may be inclined toward the radiation incident side with respect to the thin section 2007b. This can improve the workability during insertion and removal operations as well as portability.

As described above, the effective imaging area is located in the thin section 2007b, and the sloped portions 2007d are disposed on the thin end portion 2007c. The radiographic imaging apparatus 100-4 that reduces the burden felt by the subject such as a patient and improves the workability of the user during insertion and removal operations in a compatible manner can thereby be provided. The user's workability during insertion and removal can be further improved by the provision of the thick section 2007a at least at the end of the thin end portion 2007c opposite to the sloped portions 2007d.

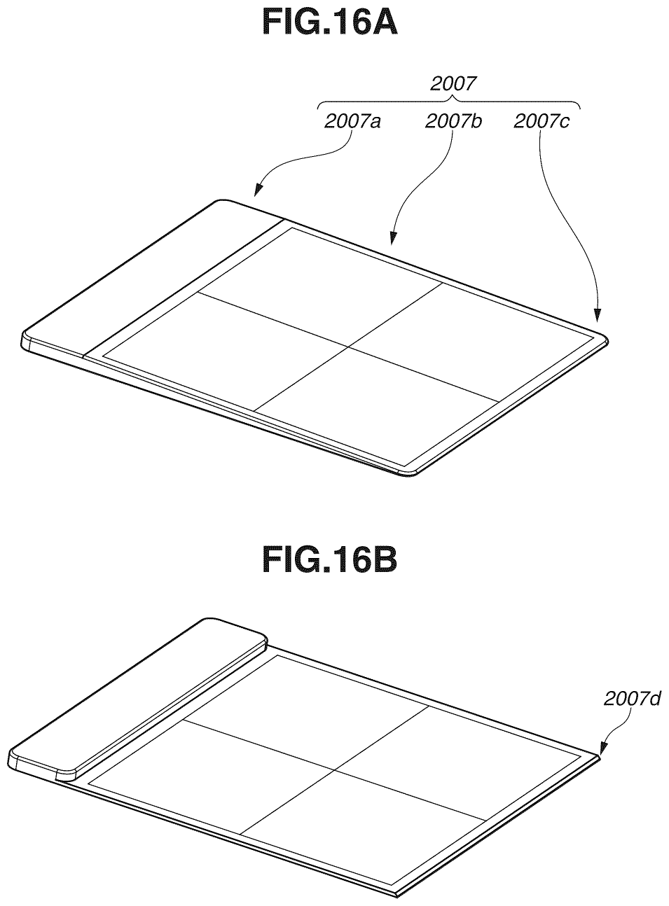

In the present exemplary embodiment, the thin section 2007b of the housing 2007 is configured to be simply flat. However, this is not restrictive, and protrusions and depressions may be formed or the outer shape may be partially changed in thickness for the purpose of improving rigidity. As illustrated in FIG. 16A, the thickness of the thin section 2007b may be gradually increased to form an outer shape continuous with the thick section 2007a.

In FIG. 16B, the sloped portions 2007d are disposed on only one side of the thin section 2007b of the housing 2007, and such a configuration may be employed. In such a case, a configuration with favorable insertion and removal workability can be provided by disposing the sloped portions 2007d on the side opposite to the thick section 2007a with respect to the center of the housing 2007 as seen in the incident direction.

The bottom surface unit of the housing 2007 has a flat structure, including the thick section 2007a. However, the thick section 2007a may be configured to protrude from the bottom. In the present exemplary embodiment, the thick section 2007a is 24 mm in thickness. However, like conventional radiographic imaging apparatuses, the thick section 2007a may be configured to be 16 mm or less.

### Fifth Exemplary Embodiment

In the present exemplary embodiment, a thin section 2007b is thinner than in the fourth exemplary embodiment. The present exemplary embodiment will be described below with reference to the drawings.

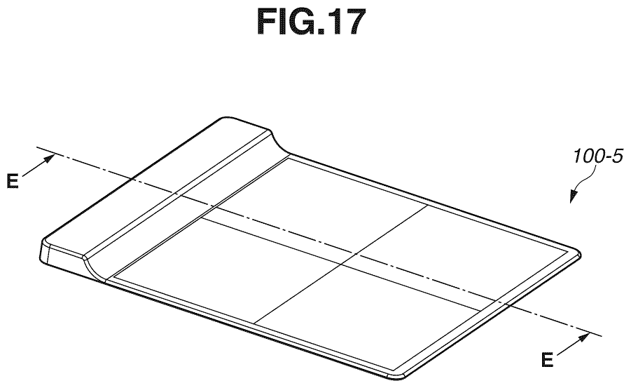

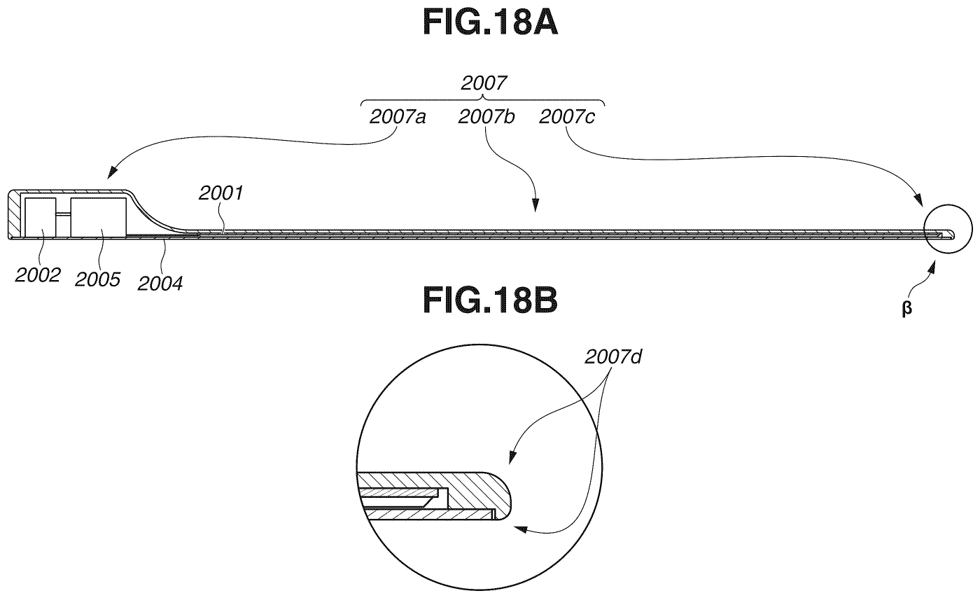

FIG. 17 is a diagram illustrating an example of the appearance of a radiographic imaging apparatus 100-5 according to a fifth exemplary embodiment. Specifically, FIG. 17 illustrates the appearance of the radiographic imaging apparatus 100-5 with a radiation detection panel 2001 built in according to the present exemplary embodiment. FIG. 18A illustrates a sectional view of the radiographic imaging apparatus 100-5 taken along line E-E illustrated in FIG. 17. FIG. 18B illustrates an enlarged view of part β in FIG. 18A. In the radiographic imaging apparatus 100-5 according to the present exemplary embodiment, as with the fourth exemplary embodiment, a housing 2007 accommodating the radiation detection panel 2001 includes a thick section 2007a that is thick in the radiation incident direction and the thin section 2007b that is thinner than the thick section 2007a. The effective imaging area of the radiation detection panel 2001 is located in the thin section 2007b as seen in the direction normal to the incident surface. A thin end portion 2007c that is an end portion of the thin section 2007b includes sloped portions 2007d. The thick section 2007a includes a battery 2002 and a control substrate 2005 that controls the radiation detection panel 2001.

Here, the radiation detection panel 2001 includes a sensor substrate and phosphor layer in order in the radiation incident direction. The sensor substrate and the inner side of the incident surface of the housing 2007 are fastened using a pressure-sensitive adhesive or the like, whereby the radiation detection panel 2001 is supported. The sensor substrate is suitably formed of flexible plastic, whereas other materials may be used.

In the present exemplary embodiment, the thin section 2007b is configured to be 4.5 mm. For such a thin configuration, the cushioning material 2003 and the support base 2006 illustrated in FIG. 12A in the fourth exemplary embodiment are omitted. The housing 2007 is fastened by a pressure-sensitive adhesive or an adhesive instead of screwing as in the fourth exemplary embodiment. The reason is that in the case of screwing like the fourth exemplary embodiment, a certain thread engagement length is needed in the radiation incident direction, which tends to be one of the factors that hinder a reduction in thickness.

Compared to screws and the like, the fastening using a pressure-sensitive adhesive or an adhesive can provide fastening force over a wide area and is likely to improve strength. Here, an adhesion structure where the adhesive surface is partially omitted may be employed.

The pressure-sensitive adhesive or the adhesive here is desirably waterproof. In view of the ease of maintenance, the pressure-sensitive adhesive or the adhesive also desirably has peelability. For the purpose of peeling, materials that drop in adhesion when exposed to heat, ultraviolet rays, or the like are suitable. Configuring the thin section 2007b thinner than in the fourth exemplary embodiment in such a manner can further reduce the reaction force caused by the contact between the subject such as a patient and the radiographic imaging apparatus 100-5 during insertion operation and during imaging.

In the present exemplary embodiment, the thin section 2007b is thinner than in the fourth exemplary embodiment. There is therefore a concern that the strength of the thin section 2007b may decrease. For that reason, in the present exemplary embodiment, the incident surface unit and the side surface unit are integrally configured. Moreover, the thickness of the side surface unit in the direction normal to the incident surface is increased to provide a wide adhesion area with the bottom surface unit and improve the rigidity of the thin section 2007b.

While the adhesive or pressure-sensitive adhesive is used in the present exemplary embodiment, a low profile may be achieved by using a screw fastening method with a reduced screw size. In such a case, when the distortion due to twisting or bending of the thin section 2007b increases, the external force is expected to be absorbed by displacement of the screw seats and the like. The screws, pressure-sensitive adhesives, and adhesives are not restrictive, and the housing 2007 may be fastened using a structure such as snap fits or fitting.

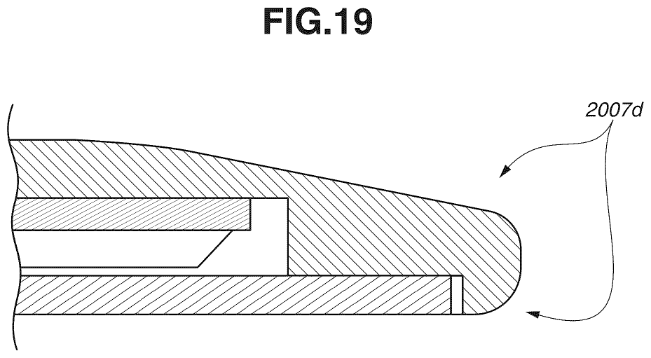

Like the fourth exemplary embodiment, the provision of the sloped portions 2007d on the thin end portion 2007c is expected to reduce the pressure due to the reaction force to the subject such as a patient. Like the fourth exemplary embodiment, the sloped portions 2007d are not limited to the round-cornered shape of FIG. 18B, either, and may have a chamfered shape such as illustrated in FIG. 19.

As seen in the direction normal to the incident surface, the sloped portions 2007d are located to overlap the radiation detection panel 2001, the effective imaging area, and the fastening portions of the housing 2007. Large sloped portions 2007d can thus be formed despite the small thickness. This also facilitates the implementation of a narrow frame structure that reduces the distance between the outline of the housing 2007 and the effective imaging area while maintaining the rigidity of the thin section 2007b. The effect of reducing discomfort of the subject such as a patient is also expected.

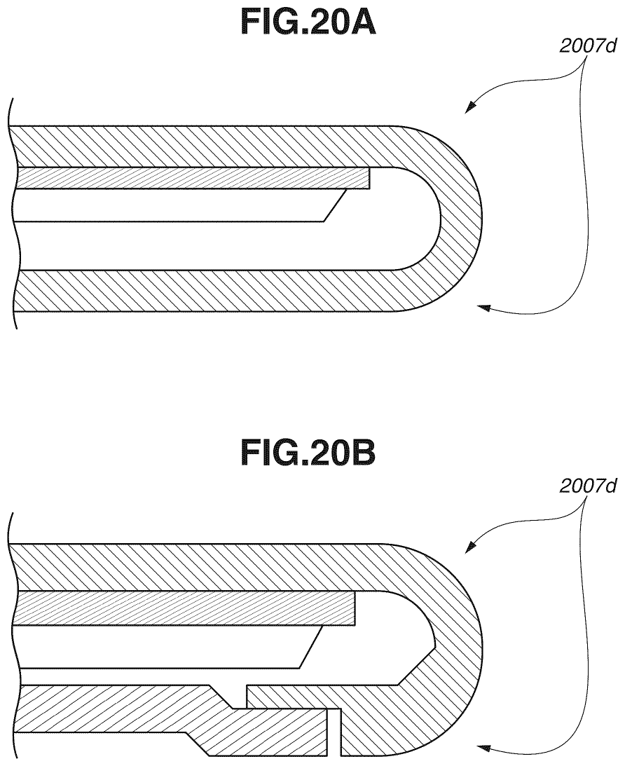

Here, the housing 2007 does not necessarily need to be configured with the components illustrated in FIGS. 18A, 18B, and 19. For example, as illustrated in FIGS. 20A, the incident surface unit, the bottom surface unit, and the side surface unit of the thin end portion 2007c may be integrally molded. In FIG. 20A, the shape is achieved by molding the thin section 2007b in a pouch-like form. Like FIG. 20B, the housing 2007 may be fastened at a location overlapping the radiation detection panel 2001 as seen in the direction normal to the incident surface. Even with such a configuration, the distance between the outline of the housing 2007 and the effective imaging area can be reduced, which facilitates the implementation of the so-called narrow frame structure.

Such an integral structure of the incident surface unit, the side surface unit, and the bottom surface unit of the thin end portion 2007c is expected to improve rigidity. Considering impact force from the end portion, the thickness of the thin end portion 2007c in the direction normal to the incident surface may be increased. This improves the strength of the thin section 2007b and can achieve the operability and strength of the radiographic imaging apparatus 100-5 in a compatible manner.

When the radiographic imaging apparatus undergoes an external force, a large stress can concentrate on the border between the thick section and the thin section. In the present exemplary embodiment, a thick sloped portion that is the border between the thick section 2007a and the thin portion 2007b is therefore shaped as a curved surface so that the thickness of the housing 2007 changes gradually. This can ease the stress concentration on the border between the thick section 2007a and the thin section 2007b.

Shaping the thick sloped portion as a curved surface can also reduce the pressure when the subject such as a patient comes into contact with the end portion of the thick section 2007a during insertion. Furthermore, to facilitate the implementation of the narrow frame structure, the thick sloped portion, and the radiation detection panel 2001 may be configured to overlap as seen in the direction normal to the incident surface.

### Sixth Exemplary Embodiment

The present exemplary embodiment deals with a mode where a thin end portion 2007c is formed of a plastic with excellent impact resistance. The present exemplary embodiment will be described below with reference to the drawings.

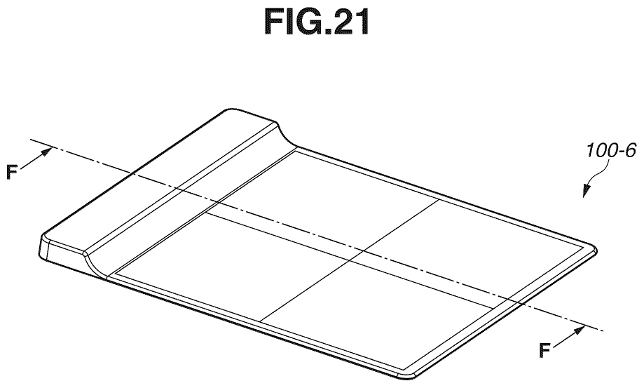

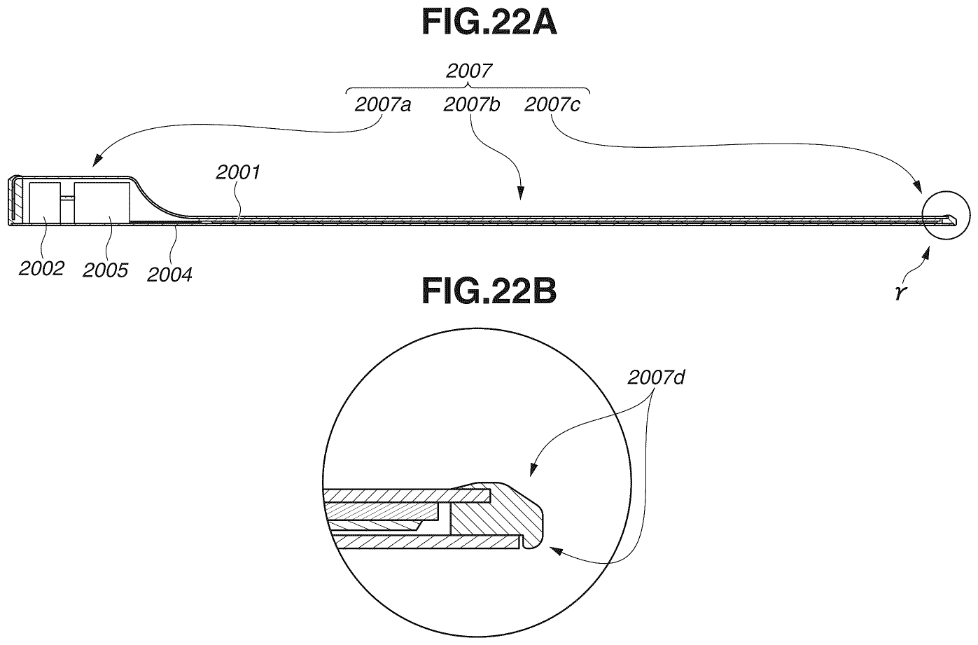

FIG. 21 is a diagram illustrating an example of the appearance of a radiographic imaging apparatus 100-6 according to a sixth exemplary embodiment. Specifically, FIG. 21 illustrates the appearance of the radiographic imaging apparatus 100-6 with a radiation detection panel 2001 built in according to the sixth exemplary embodiment. FIG. 22A illustrates a sectional view of the radiographic imaging apparatus 100-6 taken along line F-F illustrated in FIG. 21. FIG. 22B illustrates an enlarged view of part γ in FIG. 22A.

In the radiographic imaging apparatus 100-6 according to the present exemplary embodiment, like the fourth exemplary embodiment, a housing 2007 accommodating the radiation detection panel 2001 includes a thick section 2007a that is thick in the radiation incident direction and a thin section 2007b that is thinner than the thick section 2007a. As seen in the radiation incident direction, the effective imaging area of the radiation detection panel 2001 is located in the thin section 2007b. A thin end portion 2007c that is an end portion of the thin section 2007b is provided with sloped portions 2007d.

The thick section 2007a includes a battery 2002 and a control substrate 2005 that controls the radiation detection panel 2001. The thin section 2007b is configured to be 4.5 mm. The cushioning material 2003 and the support base 2006 illustrated in FIG. 12A are omitted for the sake of the configuration thinner than in the fourth exemplary embodiment, but may be provided if the outer shape is satisfied.

Here, the component that constitutes the thick section 2007a and thin section 2007b on the incident surface side of the housing 2007, the side surfaces, and the like illustrated in FIGS. 21, 22A, and 22B will be referred to as a front cover. The bottom surface opposite to the front cover will be referred to as a rear cover. The rear cover has a simple flat-plate shape and is formed of a magnesium alloy, an aluminum alloy, fiber-reinforced plastic, plastic, or the like with excellent lightweight properties, but may be otherwise configured. While the rear cover in the diagrams is a simple flat plate, protrusions and depressions may be formed or ribs may be formed for improved rigidity.

On the other hand, the periphery of the front cover is formed of a frame-shape plastic, and the other portions are formed of a thin carbon fiber-reinforced plastic (CFRP). However, the front cover may be otherwise configured. The CFRP and the surrounding frame-shaped plastic are integrally configured by using an integral molding technique such as insert molding, whereas other techniques such as adhesion may be used. The plastic is desirably one with excellent impact resistance and may be formed of materials such as elastomers, whereas other materials may also be used.

Like the fifth exemplary embodiment, the front cover and the rear cover are fastened using an adhesive or a pressure-sensitive adhesive, whereas other fastening methods may be used. Like the fourth and fifth exemplary embodiments, the sloped portions 2007d are located on the incident surface side and the bottom side of the plastic frame portion constituting the thin end portion 2007c.

In the present exemplary embodiment, as illustrated in FIG. 22B, the thin end portion 2007c is configured so that the frame-shaped plastic portion protrudes from the CFRP toward the radiation incident side as seen in a direction perpendicular to the radiation incident direction. This facilitates contact between the subject such as a patient and the frame-shaped plastic portion of the radiographic imaging apparatus 100-6 during insertion and during imaging. The plastic frame portion is formed of a material with elastic modulus lower than that of the CFRP, and is thus expected to increase the contact area of the contact portion. The pressure due to the reaction force when the subject such as a patient and the radiographic imaging apparatus 100-6 contact can thus be reduced.

Forming the thin end portion 2007c of a low-rigidity plastic or elastomer as in the present exemplary embodiment is also expected to provide an impact absorption function in the event of a drop or the like. Considering the handling of the radiographic imaging apparatus 100-6 during insertion operation, transportation, and the like, the thick section 2007a can be gripped for operation. In such a case, when the radiographic imaging apparatus 100-6 is accidentally dropped, etc., the thin end portion 2007c opposite to the thick section 2007a can be the drop impact surface. The frame-shaped plastic constituting a part of the thin end portion 2007c thus receives the drop impact, and the low elastic modulus is expected to provide the effect of reducing the acceleration peak upon the application of the impact.

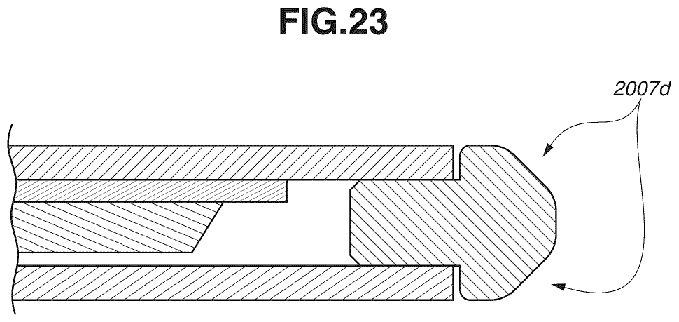

In the present exemplary embodiment, the frame-shaped plastic and the CFRP are configured as a single component by integral molding such as insert molding. However, this is not restrictive. FIG. 23 illustrates an example of a configuration where an elastic body is disposed on the thin end portion 2007c. The elastic body may be formed of rubber or elastomer, but other materials may also be used.

The elastic body is sandwiched between the incident surface side and the bottom side of the housing 2007, but may be disposed using an adhesive or the like. The rubber-sandwiching structure can also provide waterproofness. Like the plastic frame portion, an impact absorption function, and a reduction in the pressure due to the reaction force upon contact of the patient and the elastic body are also expected. Like the fourth and fifth exemplary embodiments, the elastic body is provided with sloped portions 2007d.

In the present exemplary embodiment, the thin section 2007b of the housing 2007 is configured to be simply flat. However, this is not restrictive, and protrusions and depressions may be formed or the outer shape may be partially changed in thickness for improved rigidity. Moreover, the frame-shaped plastic portion, the elastic body, and the like may be formed of materials other than those of the present exemplary embodiment.

While the preferred fourth to sixth exemplary embodiments of the present invention have been described above, these exemplary embodiments are not restrictive, and various modifications and changes can be made without departing from the gist thereof. Moreover, the foregoing exemplary embodiments may be combined as appropriate.

The fourth to sixth exemplary embodiments of the present invention include the features described in the following supplementary notes.

### [Supplementary Note 12]

A radiographic imaging apparatus including:
a radiation detection panel that includes an effective imaging area configured to detect radiation transmitted through a subject and incident on an incident surface; and
a housing that accommodates the radiation detection panel,
wherein the housing includes a thick section that is thick in a direction normal to the incident surface and located at one end of the housing, and a thin section that is thinner than the thick section and overlaps the effective imaging area at least in part as seen in the direction normal to the incident surface, and
wherein a sloped portion is disposed on at least a part of a side opposed to the thick section among a plurality of sides of the thin section, the sloped portion sloping at an end portion of the thin section.

### [Supplementary Note 13]

The radiographic imaging apparatus according to supplementary note 12, wherein a height of the sloped portion in the direction normal to the incident surface is greater than or equal to one half of a thickness of the thin section.

### [Supplementary Note 14]

The radiographic imaging apparatus according to supplementary note 12 or 13, wherein the sloped portion is disposed on at least either of the incident surface and a surface opposed to the incident surface.

### [Supplementary Note 15]

The radiographic imaging apparatus according to supplementary note 14, wherein the sloped portion is disposed on both the incident surface and the surface opposed to the incident surface.

### [Supplementary Note 16]

The radiographic imaging apparatus according to supplementary note 15, wherein a height of the sloped portion disposed on the incident surface in the direction normal to the incident surface is greater than that of the sloped portion disposed on the opposed surface.

### [Supplementary Note 17]

The radiographic imaging apparatus according to supplementary note 15, further including a side surface that connects the sloped portion disposed on the incident surface and the sloped portion disposed on the opposed surface,
wherein a height of the side surface in the direction normal to the incident surface is less than that of the sloped portion disposed on the incident surface in the direction normal to the incident surface.

### [Supplementary Note 18]

The radiographic imaging apparatus according to supplementary note 15, further including a side surface that connects the sloped portion disposed on the incident surface and the sloped portion disposed on the opposed surface,
wherein a height of the side surface in the direction normal to the incident surface is less than that of the sloped portion disposed on the opposed surface in the direction normal to the incident surface.

### [Supplementary Note 19]

The radiographic imaging apparatus according to any one of supplementary notes 12 to 18, wherein the sloped portion(s) and a part of the radiation detection panel overlap as seen in the direction normal to the incident surface.

### [Supplementary Note 20]

The radiographic imaging apparatus according to any one of supplementary notes 12 to 19, wherein the housing has a substantially polygonal shape as seen in the direction normal to the incident surface.

### [Supplementary Note 21]

The radiographic imaging apparatus according to any one of supplementary notes 12 to 20, wherein the thin section has a substantially polygonal shape as seen in the direction normal to the incident surface and includes the sloped portion on each side except for a portion adjoining the thick section.

### [Supplementary Note 22]

The radiographic imaging apparatus according to any one of supplementary notes 12 to 21, wherein the thick section includes a grip portion for gripping the radiographic imaging apparatus in an end portion opposed to a side contacting the thin section.

### [Supplementary Note 23]

The radiographic imaging apparatus according to any one of supplementary notes 12 to 22, wherein the thick section includes a thick sloped portion that slopes at an end portion of the thick section.

### [Supplementary Note 24]

The radiographic imaging apparatus according to any one of supplementary notes 12 to 23, wherein the thick section includes a thick sloped portion on a/the side contacting the thin section, the thick sloped portion serving as a curved surface connecting the thin section and the thick section.

### [Supplementary Note 25]

The radiographic imaging apparatus according to supplementary note 24, wherein the thick sloped portion is located to overlap the radiation detection panel at least in part as seen in the direction normal to the incident surface.

### [Supplementary Note 26]

The radiographic imaging apparatus according to any one of supplementary notes 12 to 25, wherein the sloped portion is formed of a material different from that of the thin section.

### [Supplementary Note 27]

The radiographic imaging apparatus according to supplementary note 26, wherein the sloped portion is formed of a material having a bending elastic modulus lower than that of at least either of a material constituting the incident surface of the thin section and a material constituting a/the surface of the thin section opposed to the incident surface.

### [Supplementary Note 28]

The radiographic imaging apparatus according to any one of supplementary notes 12 to 27, wherein the sloped portion is disposed to protrude from the incident surface of the thin section in a direction in which the radiation is incident.

### [Supplementary Note 29]

The radiographic imaging apparatus according to any one of supplementary notes 12 to 28, wherein the sloped portion and a fastening portion configured to fasten the housing are located to overlap as seen in the direction normal to the incident surface.

### [Supplementary Note 30]

The radiographic imaging apparatus according to any one of supplementary notes 12 to 29, wherein a/the fastening portion configured to fasten the housing and the radiation detection panel are located to overlap as seen in the direction normal to the incident surface.

### [Supplementary Note 31]

The radiographic imaging apparatus according to any one of supplementary notes 12 to 30, further including a control unit configured to control the radiation detection panel,
wherein at least a part of the control unit is located inside the thick section.

According to the features described in the foregoing supplementary notes 12 to 31, a radiographic imaging apparatus that improves the workability of the user and reduces the burden felt by the subject is provided.

### Seventh Exemplary Embodiment

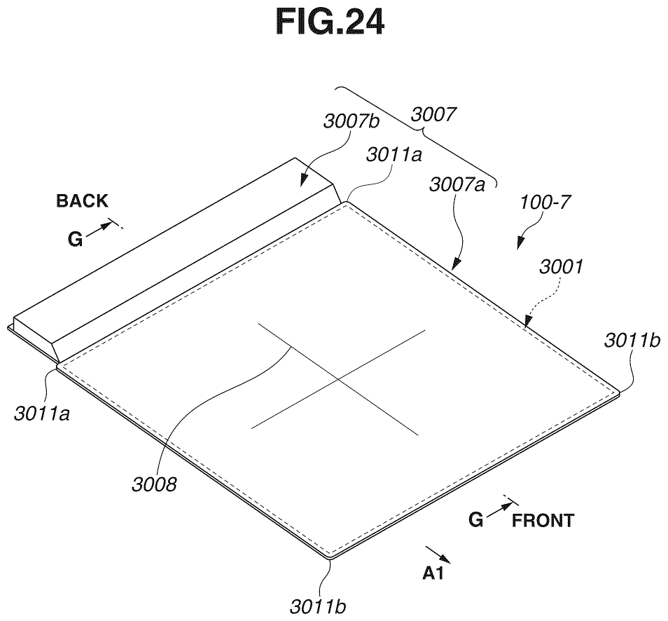

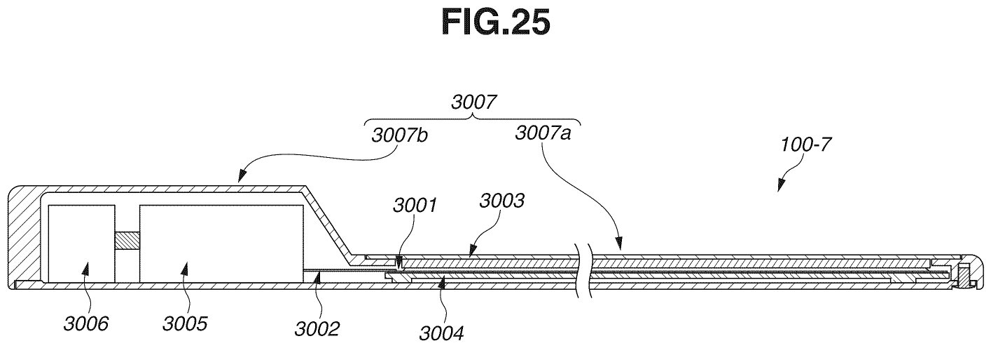

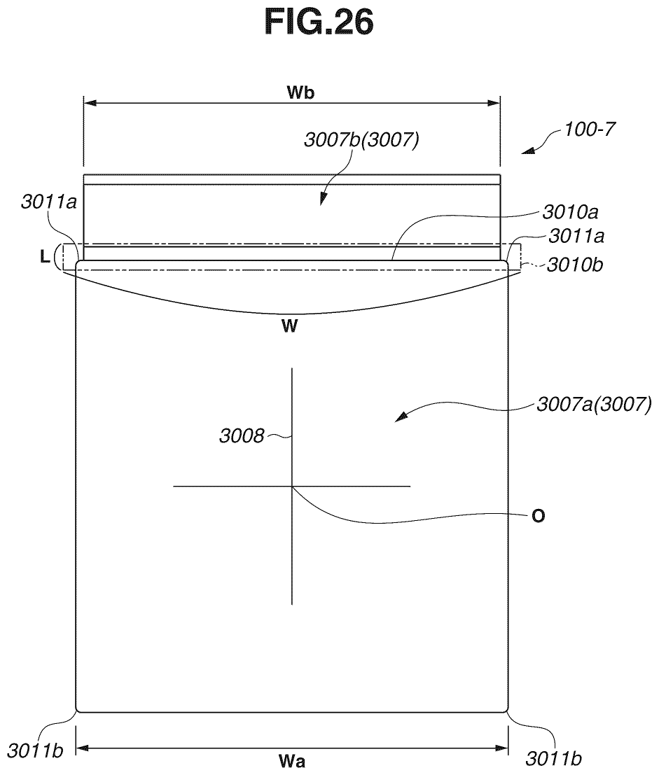

FIG. 24 is a diagram illustrating an example of the appearance of a radiographic imaging apparatus 100-7 according to a seventh exemplary embodiment. Specifically, FIG. 24 is a perspective view illustrating a configuration of the radiographic imaging apparatus 100-7. FIG. 25 is a sectional view of the radiographic imaging apparatus 100-7, and specifically a sectional view taken along line G-G illustrated in FIG. 24. FIG. 26 is a plan view of the radiographic imaging apparatus 100-7 seen in a radiation incident direction. The radiographic imaging apparatus 100-7 obtains a radiographic image by irradiating a subject with radiation from a radiation generation apparatus and detecting the radiation transmitted through the subject. The radiographic image obtained by the radiographic imaging apparatus 100-7 is transferred to outside, displayed on a monitor or the like, and used for diagnosis etc.

The radiographic imaging apparatus 100-7 includes a radiation detection panel 3001, a control substrate 3005, a battery 3006, and a housing 3007.

The radiation detection panel 3001 is of a so-called indirect conversion system, including a sensor substrate on which a large number of photoelectric conversion elements (sensors) are arranged, a phosphor layer (scintillator layer) that is located on the sensor substrate, and a phosphor protective film. The radiation detection panel 3001 includes some or all of the photoelectric conversion elements in its effective imaging area. Here, the effective imaging area refers to an area that is capable of radiographic imaging and where the radiographic image is actually generated. The effective imaging area according to the present exemplary embodiment is substantially rectangular as seen in the radiation incident direction. However, this is not restrictive. The sensor substrate is formed of a material such as glass and flexible plastic. The phosphor protective film protects the phosphor. The phosphor protective film is formed of a material with low moisture permeability to the phosphor.

Note that the radiation detection panel 3001 is not limited to the indirect conversion system and may be of a direct conversion system. The radiation detection panel of the direct conversion system includes a conversion element unit where conversion elements formed of a-Se or the like and electrical elements such as TFTs are two-dimensionally arranged. The radiation detection panel 3001 is not limited to the indirect conversion system or the direct conversion system, either.

The control substrate 3005 functions as a control unit that controls the radiation detection panel 3001. The control substrate 3005 reads detection signals from the radiation detection panel 3001 and processes the read detection signals. The control substrate 3005 is connected to the radiation detection panel 3001 via a flexible circuit board 3002. The battery 3006 supplies necessary power to the radiographic imaging apparatus 100-7. Examples of the battery 3006 include a lithium-ion battery, an electric double layer capacitor, and an all-solid-state battery.

The housing 3007 functions as an exterior casing that includes (accommodates) the radiation detection panel 3001. To achieve portability and strength in a compatible manner, the housing 3007 is formed of a magnesium alloy, an aluminum alloy, fiber-reinforced plastic, plastic, or the like. The housing 3007 includes a thin section 3007a and a thick section 3007b.

The thin section 3007a is a section of which the thickness along the radiation incident direction is less than that of the thick section 3007b. The thin section 3007a is substantially rectangular as seen in the radiation incident direction. The thin section 3007a accommodates the radiation detection panel 3001. In other words, the thin section 3007a overlaps the effective imaging area of the radiation detection panel 3001 in the radiation incident direction. A cushioning material 3003 is disposed between the incident surface of the thin section 3007a and the radiation detection panel 3001. The cushioning material 3003 protects the radiation detection panel 3001 from external force and the like. Moreover, a support base 3004 is disposed between the rear surface of the thin section 3007a and the radiation detection panel 3001. The support base 3004 supports the radiation detection panel 3001. The incident surface of the thin section 3007a is formed of a carbon fiber-reinforced plastic or the like with high radiation transmittance and excellent lightweight properties. The cushioning material 3003 is formed of a foamed resin, gel, or the like.

The incident surface of the thin section 3007a is provided with a sensor indicator 3008 for enabling recognition of the center of the effective imaging area. The sensor indicator 3008 according to the present exemplary embodiment is implemented by coloring or engraving in a cross shape, and the center of the cross indicates the center of the effective imaging area. However, the sensor indicator 3008 is not limited to the cross shape, as long as the center of the effective imaging area can be recognized.

The thick section 3007b is a section of which the thickness along the radiation incident direction is greater than that of the thin section 3007a. The thick section 3007b is substantially rectangular as seen in the radiation incident direction. The thick section 3007b is located to adjoin the thin section 3007a. Specifically, the thick section 3007b is located along one of the four sides of the rectangular shape of the thin section 3007a, and has an elongated shape long along the one side. The thick section 3007b accommodates the control substrate 3005 and the battery 3006. In other words, the thick section 3007b overlaps the control substrate 3005 and the battery 3006 in the radiation incident direction.

When imaging a subject such as a patient, the radiographic imaging apparatus 100-7 can be placed directly under the imaging site of the subject such as a patient. In doing so, a step created by the thickness of the radiographic imaging apparatus comes into contact with the subject such as a patient to cause a reaction force, and the patient such a patient may feel discomfort. The housing 3007 according to the present exemplary embodiment includes the thin section 3007a thinner than the thick section 3007b, whereby the step of the radiographic imaging apparatus 100-7 can be reduced. More specifically, by placing the thin section 3007a of the radiographic imaging apparatus 100-7 directly under the imaging site of the subject such as a patient, the reaction force occurring between the subject such as a patient and the end portion of the radiographic imaging apparatus 100-7 can be reduced to reduce the burden on the subject such as a patient. Specifically, the thin section 3007a preferably has a thickness of 10.0 mm or less, more preferably 8.0 mm or less. On the other hand, to maintain the layer configuration and the mechanical strength, the thin section 3007a is desirably 5.0 mm or thicker. To reduce the reaction force and maintain the layer structure and mechanical strength in a compatible manner, an appropriate thickness of the thin section 3007a is approximately 8 mm (+1 mm).

When imaging the subject such as a patient, the user such as a technician places the radiographic imaging apparatus 100-7 directly under the imaging site of the subject such as a patient through an insertion operation of inserting the radiographic imaging apparatus 100-7 into between the imaging site of the subject such as a patient and the bed or the like. The arrow A1 of FIG. 24 indicates the insertion direction in inserting the radiographic imaging apparatus 100-7 into between the subject such as a patient and the bed or the like, and the radiographic imaging apparatus 100-7 is inserted with the front end of the thin section 3007a ahead. Here, as illustrated in FIG. 24, the thin section 3007a side of the housing 3007 will be referred to as the front, and the thick section 3007b side as the back. In view of reducing burden and maintaining hygiene etc., towels, sheets, or other pieces of cloth may be placed over the imaging site of the subject such as a patient. During the insertion operation of the radiographic imaging apparatus 100-7, the cloth therefore covers the radiographic imaging apparatus 100-7 and makes the sensor indicator 3008 of the radiographic imaging apparatus 100-7 difficult to visually observe. With the radiographic imaging apparatus 100-7 according to the present exemplary embodiment, the center of the effective imaging area can be recognized to some degree by touching the border between the thin section 3007a and the thick section 3007b and the front end of the thin section 3007a. However, the border between the thin section 3007a and the thick section 3007b is not the area to be touched during the insertion operation, and portions for enabling the recognition of the effective imaging area therefore need to be additionally provided on the area to be touched during the insertion operation, so that the effective imaging area can be recognized simultaneously with the insertion operation.

The housing 3007 according to the present exemplary embodiment includes recognition portions that enable tactile recognition of the effective imaging area during the insertion operation. A specific configuration of the recognition portions will now be described.

The thin section 3007a according to the present exemplary embodiment is shaped to have two corner portions 3011a and two corner portions 3011b. The two corner portions 3011a and the two corner portions 3011b are portions corresponding to the four vertices (corners) of the rectangular shape of the thin section 3007a. The thin section 3007a according to the present exemplary embodiment has a width Wa greater than a width Wb of the thick section 3007b (see FIG. 26). The thin section 3007a protrudes from both sides of the thick section 3007b in the width direction, whereby the two corner portions 3011a are located outside the thick section 3007b in the width direction. The corner portions 3011a and 3011b function as the recognition portions for enabling the tactile recognition of the effective imaging area.

The corner portions 3011a are located on both sides of the border region between the thin section 3007a and the thick section 3007b in the width direction. Here, the border region between the thin section 3007a and the thick section 3007b refers to a border region 3010b indicated by the double-dotted dashed line in FIG. 26, with a border 3010a between the thin section 3007a and the thick section 3007b at the center. Here, the border region 3010b is a region having a width W greater than the width Wa of the thin section 3007a and a front-to-back length L approximately twice the thickness dimension of the thin section 3007a, for example. Note that the length L of the border region 3010b may be close to the thickness dimension of the thick section 3007b, for example, and is not limited in particular. The corner portions 3011a are located in the border region, and thus function as first recognition portions for enabling tactile recognition of the border between the thin section 3007a and the thick section 3007b.

The corner portions 3011b are located away from the border region 3010b and on both widthwise sides of the end (front end) of the thin section 3007a opposite to the thick section 3007b. Being located at the front end of the thin section 3007a opposite to the thick section 3007b, the corner portions 3011b function as second recognition portions for enabling tactile recognition of the front end of the thin section 3007a.

A center position O between the two corner portions 3011a and the two corner portions 3011b is substantially the same as the center of the effective imaging area, indicated by the sensor indicator 3008.

Since the housing 3007 is configured as described above, the corner portions 3011a can be touched while making a pushing operation in inserting the radiographic imaging apparatus 100-7 into between the subject such as a patient and the bed or the like via cloth or the like. Since the corner portions 3011a are located in the border region between the thin section 3007a and the thick section 3007b, the border between the thin section 3007a and the thick section 3007b can be recognized by touching one of the corner portions 3011a. Moreover, the center of the thin section 3007a in the width direction can be recognized by touching the two corner portions 3011a. In addition, the center of the front-to-back length of the thin section 3007a, i.e., the center of the effective imaging area can be recognized by touching the corner portions 3011b.

The corner portions 3011a and 3011b have an outer shape with curvature as seen in the radiation incident direction. In the present exemplary embodiment, the corner portions 3011a and 3011b are shaped with substantially the same curvatures. Forming the corner portions 3011a and 3011b in such an outer shape with curvature facilitates the recognition of touching the corner portions 3011a and 3011b.

As described above, according to the present exemplary embodiment, the corner portions 3011a serving as the recognition portions for enabling the recognition of the border between the thin section 3007a and the thick section 3007b are located in the border region 3010b between the thin section 3007a and the thick section 3007b. The border between the thin section 3007a and the thick section 3007b can thereby be easily recognized. Moreover, the corner portions 3011a can be touched while making a pushing operation. This can improve the workability of the radiographic imaging since the recognized thin section 3007a can be positioned directly under the imaging site of the subject such as a patient while inserting the radiographic imaging apparatus 100-7 into between the subject such as a patient and the bed or the like.

Moreover, according to the present exemplary embodiment, the width Wa of the thin section 3007a is configured to be greater than the width Wb of the thick section 3007b, whereby the corner portions 3011a capable of the pushing operation can be provided in the border region 3010b between the thin section 3007a and the thick section 3007b. Conversely, if the width Wb of the thick section 3007b is configured to be greater that the width Wa of the thin section 3007a, the corner portions are formed with inward curvature, which makes the pushing operation using the corner portions difficult. By configuring the width Wa of the thin section 3007a to be greater than the width Wb of the thick section 3007b as in the present exemplary embodiment, the corner portions 3011a can be shaped to enable both tactile recognition and the pushing operation at the same time.

### Modification 1 of Seventh Exemplary Embodiment

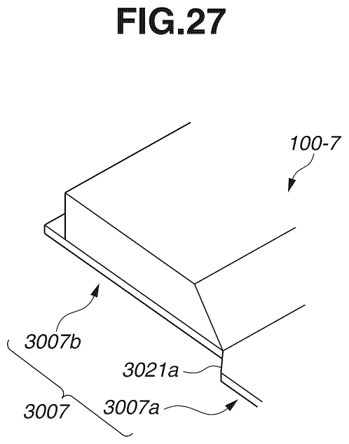

FIG. 27 is a diagram illustrating modification 1 of the corner portions of the radiographic imaging apparatus 100-7 according to the seventh exemplary embodiment. A corner portion 3021a of modification 1 illustrated in FIG. 27 has a chamfered outer shape as seen in the radiation incident direction. The chamfered outer shape of the corner portions 3021a facilitates the recognition of touching the corner portion 3021a. Not-illustrated corner portions 3021b located on both sides of the front end of the thin section 3007a in the width direction can also be configured to have a chamfered outer shape. In such a case, the corner portions 3021a and the corner portions 3021b desirably have substantially the same chamfered shape.

### Modification 2 of Seventh Exemplary Embodiment

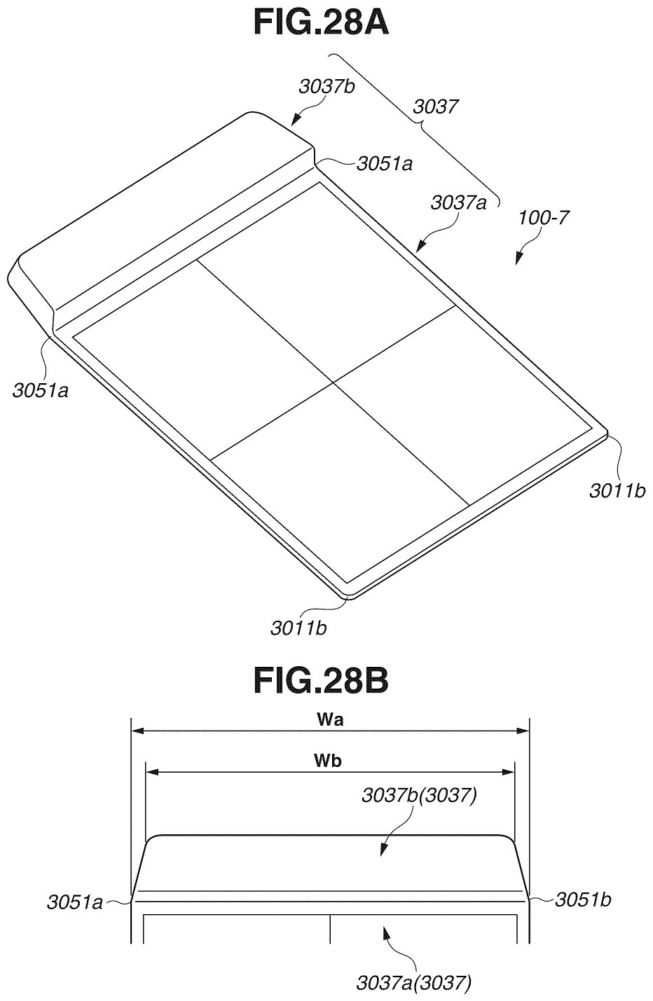

FIGS. 28A and 28B are diagrams illustrating modification 2 of the housing of the radiographic imaging apparatus 100-7 according to the seventh exemplary embodiment. A housing 3037 includes a thin section 3037a and a thick section 3037b. The thin section 3037a is substantially rectangular as seen in the radiation incident direction. The thick section 3037b is substantially trapezoidal as seen in the radiation incident direction. The thick section 3037b is located along one of the four sides of the rectangular shape of the thin section 3037a, and has an elongated shape long along the one side. The thick section 3037b has the width Wb at its back end as seen in the radiation incident direction, and slopes to increase in width toward the front side. The front end of the thick section 3037b has the same width Wa as that of the thin section 3037a. In other words, both lateral sides of the thick section 3037b are shaped to be oblique to the front-to-back direction. Both lateral sides of the thin section 3037a have a straight shape parallel to the front-to-back direction.

The thin section 3037a according to the present exemplary embodiment is shaped to have two corner portions 3051a and two corner portions 3011b. The two corner portions 3051a are located on both sides of the border region between the thin section 3037a and the thick section 3037b in the width direction. More specifically, the two corner portions 3051a are corners that are formed by connecting the lateral sides of the thick section 3037b oblique to the front-to-back direction and the straight lateral sides of the thin section 3037a parallel to the front-to-back direction and have an angle of greater than 90° and less than 180°. The two corner portions 3051a function as recognition portions for enabling tactile recognition of the effective imaging area.

### Eighth Exemplary Embodiment

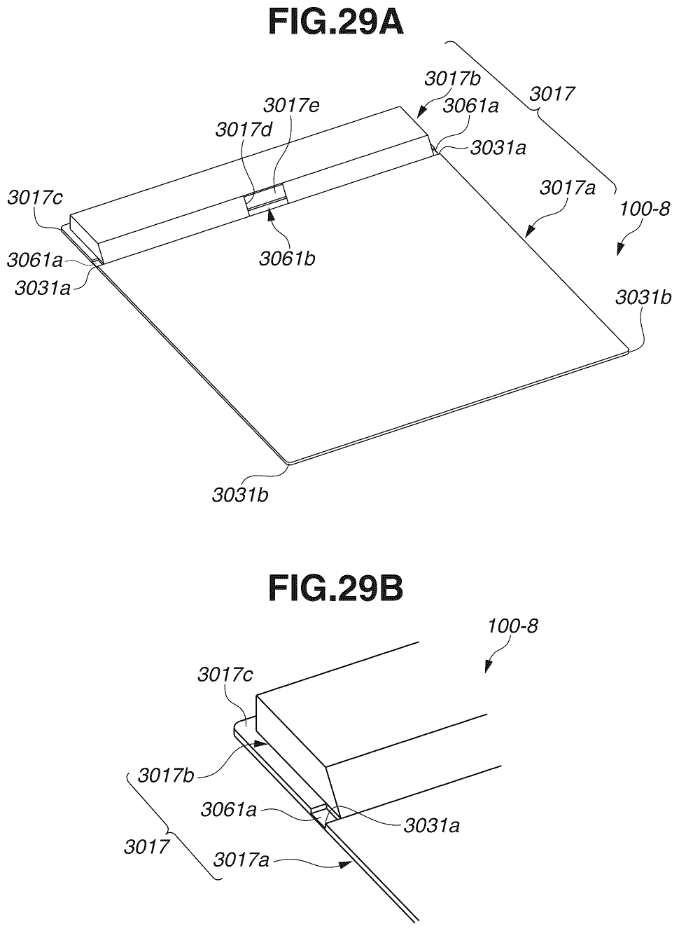

FIGS. 29A and 29B are diagrams illustrating an example of the appearance of a radiographic imaging apparatus 100-8 according to an eighth exemplary embodiment. Specifically, FIG. 29A is a perspective view illustrating a configuration of the radiographic imaging apparatus 100-8. FIG. 29B is an enlarged perspective view of a part of the configuration of the radiographic imaging apparatus 100-8. A housing 3017 includes a thin section 3017a and a thick section 3017b. The thin section 3017a according to the present exemplary embodiment is shaped to have two corner portions 3031a and two corner portions 3031b. The two corner portions 3031a and the two corner portions 3031b are portions corresponding to the four vertices (corners) of the rectangular shape of the thin section 3017a. The thin section 3017a according to the present exemplary embodiment has substantially the same width as that of the thick section 3017b. The housing 3017 includes a flange portion 3017c around the thick section 3017b except for the thin section 3017a side. The thickness of the flange portion 3017c along the radiation incident direction is substantially the same as that of the thin section 3017a, for example.

The housing 3017 according to the present exemplary embodiment includes grooves 3061a in the border region between the thin section 3017a and the thick section 3017b. The grooves 3061a function as recognition portions for enabling recognition of the border between the thin section 3017a and the thick section 3017b. Here, the grooves 3061a have a groove shape recessed along the radiation incident direction. The grooves 3061a are located to adjoin the two respective corner portions 3031a. More specifically, the grooves 3061a are located on both sides of the thick section 3017b in the width direction and between the corner portions 3031a of the thin section 3017a and the flange portion 3017c.

Here, the bottom of the grooves 3061a have a thickness less than that of the thin section 3017a. The grooves 3061a are therefore recessed from the top surface of the thin section 3017a in the radiation incident direction. In inserting the radiographic imaging apparatus 100-8 into between a subject such as a patient and a bed or the like via cloth or the like, the grooves 3061a can be touched during the pushing operation. Since the grooves 3061a are located in the border region between the thin section 3017a and the thick section 3017b, the border between the thin section 3017a and the thick section 3017b can be recognized by touching one of the grooves 3061a. Moreover, the center of the thin section 3017a in the width direction can be recognized by touching the two grooves 3061a. In addition, the center of the thin section 3017a in the front-to-back direction, i.e., the center of the effective imaging area can be recognized by touching the corner portions 3031b.

The housing 3017 according to the present exemplary embodiment also has a groove 3061b in the border region between the thin section 3017a and the thick section 3017b and between the two corner portions 3031a, or more specifically, substantially in the center therebetween. A cutout 3017d is formed substantially in the center of the thick section 3017b in the width direction, and the groove 3061b can be touched through the cutout 3017d. In FIGS. 29A and 29B, an inner cover 3017e for protecting the control substrate 3005 to not be touched through the cutout 3017d is disposed inside the thick section 3017b.

The bottom of the groove 3061b has a thickness less than that of the thin section 3017a. The groove 3061b is therefore recessed from the top surface of the thin section 3017a in the radiation incident direction. In inserting the radiographic imaging apparatus 100-8 into between the subject such as a patient and the bed or the like via cloth or the like, the groove 3061b can thus be touched during the pushing operation. Since the groove 3061b is located in the border region between the thin section 3017a and the thick section 3017b and substantially in the center in the width direction, the border between the thin section 3017a and the thick section 3017b and the center of the thin section 3017a in the width direction can be recognized by touching the groove 3061b.

The provision of the grooves 3061a and 3061b in the housing 3017 as in the present exemplary embodiment can further facilitate the recognition of the border between the thin section 3017a and the thick section 3017b, and facilitate the pushing operation in each direction.

The housing 3017 is not limited to the grooves 3061a and 3061b, and may include only the grooves 3061a. The groove 3061b is not limited to being located in the border region between the thin section 3017a and the thick section 3017b and substantially in the center in the width direction. A groove 3061b of straight shape may be formed at the border between the thin section 3017a and the thick section 3017b, from one end to the other in the width direction. A plurality of grooves 3061a may be intermittently formed.

### Modification 1 of Eighth Exemplary Embodiment

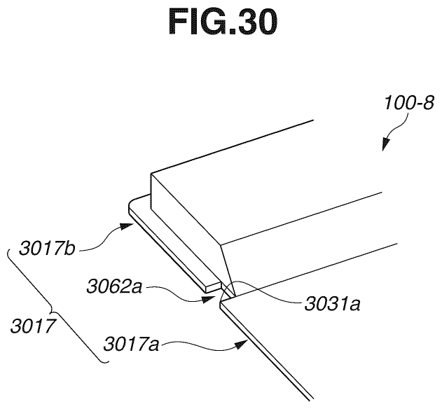

FIG. 30 is a diagram illustrating modification 1 of a groove of the radiographic imaging apparatus 100-8 according to the eighth exemplary embodiment. A groove 3062a of modification 1 illustrated in FIG. 30 is a through hole that runs through in the radiation incident direction. Configurating the groove 3062a as a through hole can facilitate the pushing operation. Configuring the groove 3062a as a through hole can also facilitate recognition of touching the groove 3062a. If there is a groove 3062b in the border portion between the thin section 3017a and the thick section 3017b and substantially in the center between the two corner portions 3031a, the groove 3062b may also be configured as a through hole.

### Ninth Exemplary Embodiment

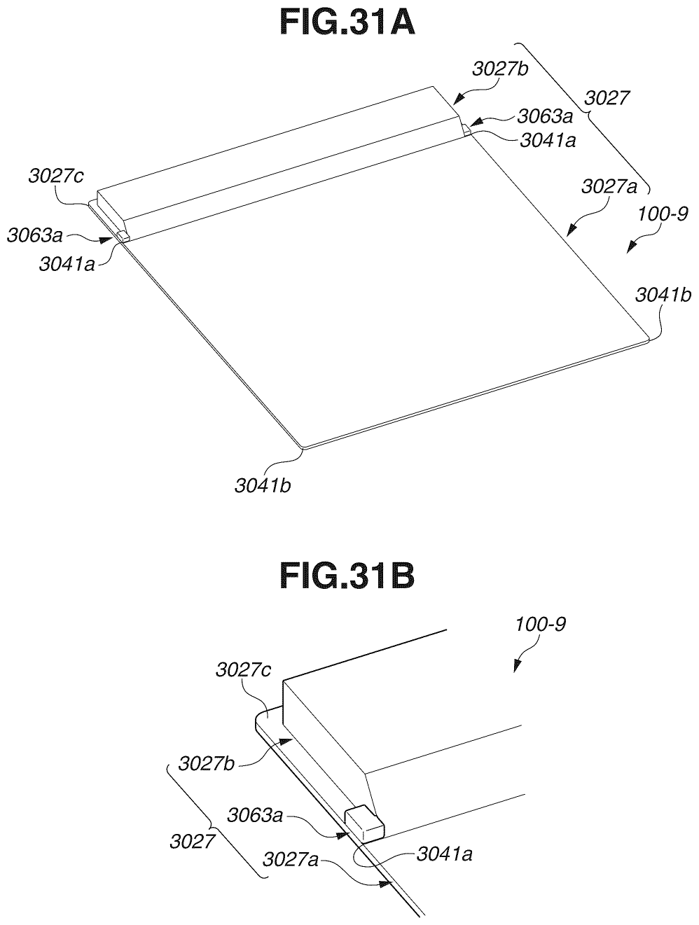

FIGS. 31A and 31B are diagrams illustrating an example of the appearance of a radiographic imaging apparatus 100-9 according to a ninth exemplary embodiment. Specifically, FIG. 31A is a perspective view illustrating a configuration of the radiographic imaging apparatus 100-9. FIG. 31B is an enlarged perspective view of a part of the configuration of the radiographic imaging apparatus 100-9. A housing 3027 includes a thin section 3027a and a thick section 3027b. The thin section 3027a according to the present exemplary embodiment has two corner portions 3041a and two corner portions 3041b. The two corner portions 3041a and the two corner portions 3041b are portions corresponding to the fourth vertices (corners) of the rectangular shape of the thin section 3027a. The thin section 3027a according to the present exemplary embodiment has substantially the same width as that of the thick section 3027b. Note that the housing 3027 includes a flange portion 3027c around the thick section 3027b except for the thin section 3027a side. The thickness of the flange portion 3027c along the radiation incident direction is substantially the same as that of the thin section 3027a, for example.

The housing 3027 according to the present exemplary embodiment includes protrusions 3063a in the border region between the thin section 3027a and the thick section 3027b. The protrusions 3063a function as recognition portions for enabling recognition of the border between the thin section 3027a and the thick section 3027b. Here, the protrusions 3063a have a protruding shape protruding along the radiation incident direction. The protrusions 3063a are located to adjoin the two respective corner portions 3041a. More specifically, the protrusions 3063a are located on both sides of the thick section 3027b in the width direction and between the corner portions 3041a of the thin section 3027a and the flange portion 3027c.

Here, the protrusions 3063a have a thickness greater than that of the thin section 3027a and less than that of the thick section 3027b. The protrusions 3063a therefore protrude from the top surface of the thin section 3027a in a direction opposite to the radiation incident direction. In inserting the radiographic imaging apparatus 100-9 into between a subject such as a patient and a bed or the like via cloth or the like, the protrusions 3063a can be touched during the pushing operation. Since the protrusions 3063a are located in the border region between the thin section 3027a and the thick section 3027b, the border between the thin section 3027a and the thick section 3027b can be recognized by touching one of the protrusions 3063a. Moreover, the center of the thin section 3027a in the width direction can be recognized by touching the two protrusions 3063a. In addition, the center of the thin section 3027a in the longitudinal direction, i.e., the center of the effective imaging area can be recognized by touching the corner portions 3041b.

Note that the housing 3027 may include a protrusion in the border region between the thin section 3027a and the thick section 3027b and between the two corner portions 3041a, or more specifically, substantially in the center therebetween, but the thick section 3027b can interfere with the installation. The protrusions 3063a are therefore desirably disposed only at the two corner portions 3041a.

### Modification 1 of Ninth Exemplary Embodiment

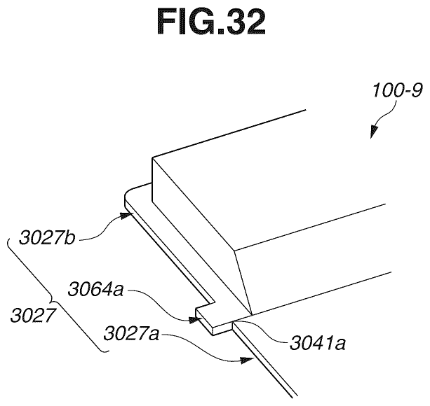

FIG. 32 is a diagram illustrating modification 1 of the protrusions of the radiographic imaging apparatus 100-9 according to the ninth exemplary embodiment. A protrusion 3064a of modification 1 illustrated in FIG. 32 has a protruding shape protruding in a direction orthogonal to the radiation incident direction. Specifically, the protrusion 3064a protrudes from the thin section 3027a and the thick section 3027b in the width direction. Configuring the protrusion 3064a to protrude in the width direction facilitates the pushing operation. Configuring the protrusion 3064a to protrude in the width direction also facilitates recognition of touching with the protrusion 3064a.

While the preferred seventh to ninth exemplary embodiments of the present invention have been described above, these exemplary embodiments are not restrictive, and various modifications and changes can be made without departing from the gist thereof. Moreover, the foregoing exemplary embodiments may be combined as appropriate.

The seventh to ninth exemplary embodiments of the present invention include the features described in the following supplementary notes.

### [Supplementary Note 32]

A radiographic imaging apparatus including:
a radiation detection panel that includes an effective imaging area configured to detect radiation transmitted through a subject, the subject being irradiated with radiation from a radiation generation apparatus; and
a housing that accommodates the radiation detection panel,
wherein the housing includes
a thin section that overlaps the effective imaging area in a radiation incident direction,
a thick section that is thicker than the thin section along the radiation incident direction, and
a recognition portion that is located in a border region between the thin section and the thick section and configured to enable recognition of a border between the thin section and the thick section.

### [Supplementary Note 33]

The radiographic imaging apparatus according to supplementary note 32, further including a control unit configured to control the radiation detection panel,
wherein the control unit is disposed inside the thick section.

### [Supplementary Note 34]

The radiographic imaging apparatus according to supplementary note 32 or 33,
wherein the thin section is shaped to include a corner portion in the border region, and
wherein the recognition portion is the corner portion and has an outer shape with curvature as seen in the radiation incident direction.

### [Supplementary Note 35]

The radiographic imaging apparatus according to any one of supplementary notes 32 to 34,
wherein the thin section is shaped to include two corner portions in the border region, and
wherein the recognition portion is the two corner portions and has an outer shape with curvature as seen in the radiation incident direction.

### [Supplementary Note 36]

The radiographic imaging apparatus according to any one of supplementary notes 32 to 35,
wherein the thin section includes four corner portions including (the) two corner portions in the border region and two corner portions away from the border region,
wherein, with the recognition portion as a first recognition portion, the two corner portions in the border region serve as the first recognition portion, and the two corner portions away from the border region serve as a second recognition portion, and
wherein the first and second recognition portions are shaped to have outer shapes with substantially same curvatures as seen in the radiation incident direction.

### [Supplementary Note 37]

The radiographic imaging apparatus according to supplementary note 32 or 33,
wherein the thin section is shaped to include a corner portion in the border region, and
wherein the recognition portion is the corner portion and has a chamfered outer shape as seen in the radiation incident direction.

### [Supplementary Note 38]

The radiographic imaging apparatus according to any one of supplementary notes 32 to 37, including, with the recognition portion as a/the first recognition portion, two first recognition portions in the border region and two second recognition portions away from the border region,
wherein a center position between the two first recognition portions and the two second recognition portions as seen in the radiation incident direction is substantially same as a center of an indicator of the effective imaging area.

### [Supplementary Note 39]

The radiographic imaging apparatus according to supplementary note 32 or 33, wherein the recognition portion has a groove shape recessed along the radiation incident direction.

### [Supplementary Note 40]

The radiographic imaging apparatus according to supplementary note 39,
wherein the thin section is shaped to include two corner portions in the border region, and
wherein the recognition portion is located only at each of the two corner positions.

### [Supplementary Note 41]

The radiographic imaging apparatus according to supplementary note 39,
wherein the thin section is shaped to include two corner portions in the border region, and
wherein the recognition portion is located at each of the two corner portions, as well as between the two corner portions in the border region.

### [Supplementary Note 42]

The radiographic imaging apparatus according to supplementary note 41, wherein a plurality of recognition portions is disposed between the two corner portions in the border region.

### [Supplementary Note 43]

The radiographic imaging apparatus according to any one of supplementary notes 39 to 42, wherein the groove shape is a through hole that runs through in the radiation incident direction.

### [Supplementary Note 44]

The radiographic imaging apparatus according to supplementary note 32 or 33, wherein the recognition portion has a protruding shape.

### [Supplementary Note 45]

The radiographic imaging apparatus according to supplementary note 44,
wherein the thin section is shaped to include two corner portions in the border region, and
wherein the recognition portion is located only at each of the two corner portions.

### [Supplementary Note 46]

The radiographic imaging apparatus according to supplementary note 44 or 45, wherein the protruding shape protrudes along the radiation incident direction.

### [Supplementary Note 47]

The radiographic imaging apparatus according to any one of supplementary notes 44 to 46, wherein a thickness of the protruding shape along the radiation incident direction is different from that of the thick section.

### [Supplementary Note 48]

The radiographic imaging apparatus according to any one of supplementary notes 44 to 47, wherein a/the thickness of the protruding shape along the radiation incident direction is greater than that of the thin section.

### [Supplementary Note 49]

The radiographic imaging apparatus according to any one of supplementary notes 32 to 48, wherein the thin section has a thickness of 10.0 mm or less.

According to the features described in the foregoing supplementary notes 32 to 49, the burden on the subject such as a patient can be reduced and the effective imaging area can be easily recognized.

### Tenth Exemplary Embodiment

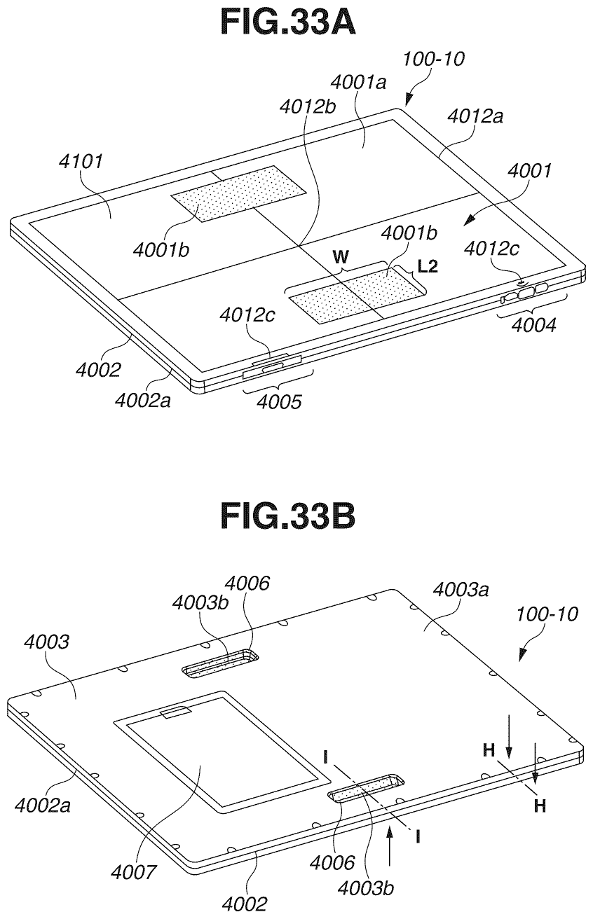

FIGS. 33A and 33B are diagrams illustrating an example of the appearance of a radiographic imaging apparatus 100-10 according to a tenth exemplary embodiment. Specifically, FIG. 33A is a perspective view as seen from the front side. FIG. 33B is a perspective view as seen from the rear side. The radiographic imaging apparatus 100-10 includes a housing 4101 of low-profile box shape that constitutes an outer casing thereof. The housing 4101 is configured by combining a front cover 4001 that constitutes the front surface, a rear cover 4003 that constitutes the rear surface opposed to the front surface, and a frame 4002 that is connected to the front cover 4001 and the rear cover 4003 and constitutes side surfaces connecting the front surface and the rear surface. The front surface, the side surfaces, and the rear surface will hereinafter be also denoted by the reference numerals 4001, 4002, and 4003, respectively. In view of radiation transmittance and light weight, carbon fiber-reinforce plastic (CFRP) or a magnesium alloy is used as the material of the housing 4101. While the housing 4101 is described to be configured by combining the front cover 4001, the rear cover 4003, and the frame 4002, some of these may be integrated, for example.

The front surface 4001 of the housing 4101 constitutes an incident surface of radiation such as X-rays. The front surface 4001 is provided with a line-shaped indicator 4012a indicating an imaging area, and a line-shaped indicator 4012b indicating the center of the imaging area. Indicators 4012c, such as characters, that indicate the positions of a user interface 4004 and a connector 4005 located on a side surface 4002 are provided near an end portion of the front surface 4001. A battery compartment 4007 for supplying power and grip portions 4006 for facilitating the user to hold the radiographic imaging apparatus 100-10 are disposed in the rear surface 4003 of the housing 4101. The grip portions 4006 are recesses designed to place fingers in, and arranged along the sides of the housing 4101. The user interface 4004 including a power switch, an LED indicating the remaining battery level, and a ready switch indicating an imaging preparation state, and the connector 4005 intended for cable connection are located on the side surface 4002 of the housing 4101.

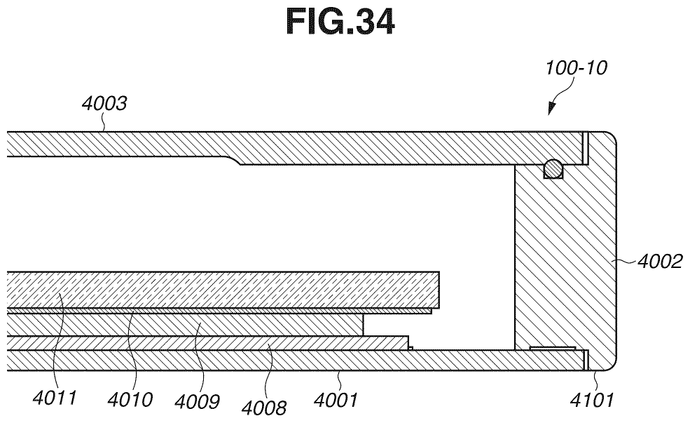

An internal configuration of the radiographic imaging apparatus 100-10 will be described with reference to FIG. 34. FIG. 34 is a sectional view of the radiographic imaging apparatus 100-10 taken along line H-H illustrated in FIG. 33B. An impact absorption sheet 4008, a radiation detection panel 4009, a radiation shielding sheet 4010, a support base 4011, and a not-illustrated battery, control substrate, antenna, and the like are accommodated and installed inside the housing 4101 in order from the front surface 4001 side. The radiation detection panel 4009 is of a so-called indirect conversion system, including a sensor substrate where a large number of photoelectric conversion elements (sensors) are arranged, a phosphor layer (scintillator layer) that is located on the sensor substrate, and a phosphor protective film. The radiation detection panel 4009 includes a part or all of the area where the photoelectric conversion elements are arranged as its imaging area. The imaging area is an area that is capable of radiographic imaging and where a radiographic image is actually generated. The phosphor protective film is formed of a material with low moisture permeability and used to protect the phosphor. Such a radiation detection panel 4009 is connected to the control substrate via a flexible circuit board. The control substrate reads detection signals from the radiation detection panel 4009 and processes the read detection signals. Note that the radiation detection panel is not limited to the indirect conversion system, and may be of so-called direct conversion type, including a conversion element unit where conversion elements formed of a-Se or the like and electrical elements such as TFTs are two-dimensionally arranged, for example. Examples of the material of the sensor substrate of the radiation detection panel 4009 include, but not limited to, glass and flexible plastic.

With such a radiographic imaging apparatus 100-10, radiation emitted from a not-illustrated radiation generation apparatus and transmitted through a subject is incident on the front surface 4001, and is detected by the radiation detection panel 4009. The radiographic image obtained by the radiographic imaging apparatus 100-10 is transferred to outside, displayed on a monitor or the like, and used for diagnosis etc.

A low-friction region 4001a given low-friction treatment is provided on the front surface 4001 of the housing 4101. In the example illustrated in FIG. 33A, the area other than non-low-friction regions 4001b to be described below is the low-friction region 4001a. In FIG. 33A, the non-low-friction regions 4001b are illustrated with a dot pattern, which is applied for the sake of convenience in illustration. The front surface 4001 is the part that contacts the subject such as a patient when the radiographic imaging apparatus 100-10 is placed under the subject such as a patient lying on the bed. The provision of the low-friction region 4001a here facilitates the movement of the radiographic imaging apparatus 100-10 in inserting, removing, and positioning the radiographic imaging apparatus 100-10. The low-friction region 4001a is desirably disposed over a wide area of the front surface 4001, and particularly over the imaging area corresponding to the radiation detection panel 4009 for easier movement.

A low-friction region 4003a given low-friction treatment is also provided on the rear surface 4003 of the housing 4101. In the example illustrated in FIG. 33B, the area excluding non-low-friction regions 4003b to be described below is the low-friction region 4003a. Again, in FIG. 33B, the non-low-friction regions 4003b are illustrated with a dot pattern, which is applied for the sake of convenience in illustration. The rear surface 4003 is the part that contacts the bed sheet when the radiographic imaging apparatus 100-10 is placed under the subject such as a patient lying on the bed. The provision of the low-friction region 4003a here facilitates the movement of the radiographic imaging apparatus 100-10 in inserting, removing, and positioning the radiographic imaging apparatus 100-10. The low-friction region 4003a is desirably disposed over a wide area of the rear surface 4003.

A low-friction region 4002a given low-friction treatment is provided on the side surfaces 4002 of the housing 4101. In the example illustrated in FIGS. 33A and 33B, the entirety of the side surfaces 4002 is the low-friction region 4002a. The provision of the low-friction region 4002a here can reduce catching on the patient gown, sheet, and the like when the radiographic imaging apparatus 100-10 is moved.

One of the indexes that indicate the degree of friction force is the coefficient of kinetic friction. The low-friction regions 4001a to 4003a have a coefficient of kinetic friction lower than that of the CFRP or magnesium alloy that is the material of the housing 4101, and is 0.15 or less, suitably 0.10 or less. An insertability/removability test of the radiographic imaging apparatus 100-10 was conducted, simulating a condition where an adult male is lying on a bed. As a result, the radiographic imaging apparatus 100-10 was difficult to push in with only the material of the housing 4101 (CFRP or magnesium alloy). By contract, at a coefficient of kinetic friction of 0.15 or less, the radiographic imaging apparatus 100-10 was successfully pushed in with light force. In particular, at a coefficient of kinetic friction of 0.10 or so, the radiographic imaging apparatus 100-10 was successfully pushed in by even a female operator. The coefficient of kinetic friction was measured using test pieces of CFRP or magnesium alloy sheets given low-friction treatment, by moving the test pieces at a speed of 30 mm/s under a load of 500 g·f with a stainless (SUS) ball (with a diameter of 3.0 mm) as the counter material.

While various methods can be used for low-friction treatment, the low-friction regions 4001a to 4003a in the present exemplary embodiment are formed by applying paint or ink (in the following description, referred to as “paint”) to the housing 4101. Since the housing 4101 has not only flat surfaces but also protrusions, depressions, R sections, and other shapes, applying paint is more suitable than attaching a sheet material, for example. Paint is particularly suitable for applying low-friction treatment to edges at the ends of parts, grooves between adjacent parts, etc. Low-friction regions on smooth surfaces or gentle protrusions and depressions may be formed using sheet materials. Paint and sheet materials may be used in combination.

Various characteristics are required of the housing 4101, including chemical resistance, abrasion resistance, and the absence of adverse effects on the human body. Paint compounded with materials containing urethane bonds has been found to be suitable for achieving these characteristics and low friction. Urethane groups have strong cohesive force and can reduce friction force. In the present exemplary embodiment, urethane or acrylic urethane paint is used. Teflon (registered trademark) and other fluorine material-based paints can also be used to achieve low friction, whereas fluorine-based paints need a high-temperature baking treatment and applicable locations are limited.

Fine particles called beads may be compounded with the paint for reduced friction. By compounding fine particles, low friction can be achieved with an improvement in abrasion resistance, which is suitable for the radiographic imaging apparatus 100-10 that is inserted and removed repeatedly. Favorable fine particle materials include urethane-, silicone-, and fluorine-based resins, metallic soaps, and inorganic materials such as silica and carbon. When forming the low-friction regions, primers can be used to improve the adhesion of the paint. The types of primers are not limited in particular. For a paint containing urethane groups, primers containing urethane-based materials are favorably used.

Considering the use of the radiographic imaging apparatus 100-10 in medical settings, the paint may be compounded with materials having antibacterial effects. Examples of the materials with antibacterial effects include metal-based antibacterial agents based on Ag, Ti, Cu, and the like, and organic antibacterial agents.

Next, the non-low-friction regions 4001b and 4003b will be described. Non-low-friction regions are areas where the coefficient of kinetic friction is higher than that of low-friction regions. Examples include areas where the material of the housing 4101 is left intact without low-friction treatment, and high-friction regions given high-friction treatment. With low-friction regions, the radiographic imaging apparatus 100-10 (housing 4101) gripped by the user is more likely to slip off, and the slipped radiographic imaging apparatus 100-10 can fall and hit on the user or be damaged. Therefore, instead of configuring the entire surface of the housing 4101 as a low-friction region, non-low-friction regions are provided in part.

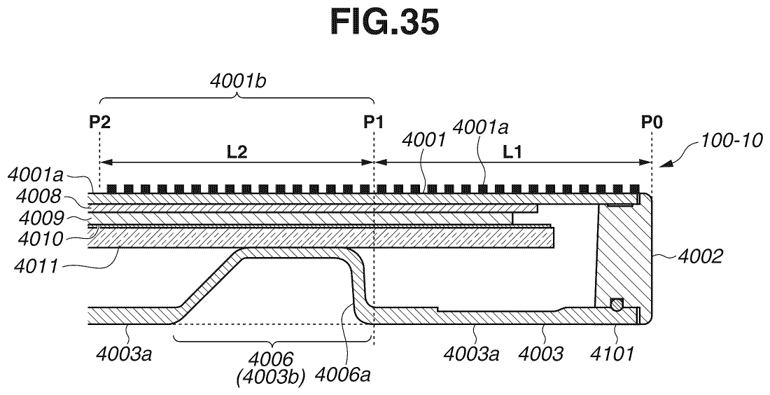

An example of the non-low-friction regions 4001b and 4003b will be described with reference to FIG. 35. FIG. 35 is a sectional view of the radiographic imaging apparatus 100-10 taken along line I-I illustrated in FIG. 33B. In the present exemplary embodiment, the grip portions 4006 in the rear surface 4003 of the housing 4101 are configured as the non-low-friction regions 4003b. The reason is that the user often holds the radiographic imaging apparatus 100-10 and manipulates the radiographic imaging apparatus 100-10 with fingers in the grip portions 4006 that are recesses. Such grip portions 4006 to place fingers in are desirably configured as high-friction regions given high-friction treatment, and suitably have a coefficient of kinetic friction of 0.50 or more. The high-friction regions are formed by applying a rubber-based paint with high friction force or disposing a self-adhesive material.

Predetermined areas corresponding to the grip portions 4006 on the front surface 4001 of the housing 4101 are configured as the non-low-friction regions 4001b. The user pinches and holds the housing 4101 with the thumbs in the grip portions 4006 in the rear surface 4003 and the other fingers in contact with the front surface 4001 across the side surfaces 4002, for example. Alternatively, the user pinches and holds the housing 4101 with the fingers other than the thumbs in the grip portions in the rear surface 4003 and the thumbs in contact with the front surface 4001 across the side surfaces 4002, for example. Configuring the areas where the user's fingers or thumbs contact on the front surface 4001 as the non-low-friction regions 4001b, or desirably high-friction regions with a coefficient of kinetic friction of 0.50 or more, thus enables a firm grip.

More specifically, as illustrated in FIG. 35, on the front surface 4001 of the housing 4101, the position of the grip portion 4006 close to the end portion (side surface 4002) of the housing 4101 will be referred to as a position P1. Assume a range of a distance L1 from an end position P0 of the housing 4101 to the position P1, and a range of a distance L2 inward from the position P1. The user pinches and holds the housing 4101 with the fingers or the thumb on the side surface (side surface closer to the end portion of the housing 4101) 4006a of the grip portion 4006 and the thumb or the other fingers in contact with the front surface 4001 across the side surface 4002. Considering this, the distance L1 is suitably set to 25 mm to 40 mm or so. The user's thumb or fingers is/are expected to contact the front surface 4001 further inward of the position P1. To accommodate typical finger lengths, the length L2 is suitably set to 100 mm or so.

In the present exemplary embodiment, the range of the distance L2 inward from the position P1 is configured as a non-low-friction region (high-friction region) 4001b. A width W (see FIG. 33A) of the non-low-friction region (high-friction region) 4001b can be set as appropriate, and is preferably set to a width that allows contact with the user's four fingers other than the thumb. In the present exemplary embodiment, the range of the distance L1 from the end position P0 to the position P1 is configured as a low-friction region 4001a, whereas this range may also be configured as a non-low-friction region (high-friction region) 4001b.

As described above, the low-friction regions 4001a and 4003a and the non-low-friction regions 4001b and 4003b coexist on the front surface 4001 and the rear surface 4003. In particular, the front surface 4001 is configured so that the regions with different coefficients of kinetic friction are formed on substantially the same plane.

Not only the grip portions 4006 but other recesses may also be configured as non-low-friction regions. Since recesses are less likely to contact the subject such as a patient or the sheet and the like, the non-low-friction regions are unlikely to interfere with the movement of the radiographic imaging apparatus 100-10. In the present exemplary embodiment, two grip portions 4006 are described to be formed. However, a grip portion 4006 including one or more recesses may be formed and disposed along each of the four sides. The recesses or grip portions 4006 along the respective sides may be connected to form an annular grip portion 4006. Instead of configuring the grip portions 4006 as recesses, the grip portions 4006 may be formed in a handle-like shape with a hole that runs through the front surface 4001 and the rear surface 4003. In such a case, a low-friction region is disposed at least on part of the handle-like shape.

As described above, the front surface 4001 and the rear surface 4003 of the housing 4101 are provided with the low-friction regions 4001a and 4003a having a coefficient of kinetic friction of 0.15 or less. This can reduce the force needed to move the radiographic imaging apparatus 100-10 under the subject such as a patient lying on the bed. Moreover, the subject such as a patient is less likely to experience pain from abrasion when the radiographic imaging apparatus 100-10 is moved. The radiographic imaging apparatus 100-10 that can improve workability and reduce burden on the subject such as a patient can thus be provided.

The low-friction regions described in FIGS. 33A to 35 are just an example and not restrictive. At least either one of, and preferably both of, the front surface 4001 and the rear surface 4003 can be provided with low-friction regions. For example, while the entirety of the side surfaces 4002 of the housing 4101 is described to be a low-friction region 4002a, the entirety may be configured as a non-low-friction region. Low-friction and non-low-friction regions may coexist. The provision of a non-low-friction region on the side surfaces 4002 of the housing 4101 can improve the ease of holding. Non-low-friction regions may be disposed around the user interface 4004 and the connector 4005 on the side surface 4002 of the housing 4101 that are expected to be touched by the user.

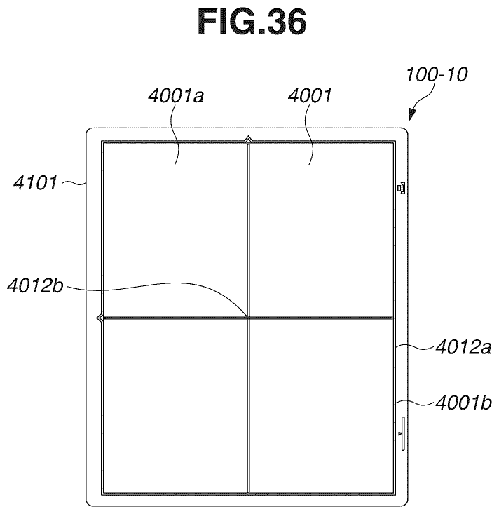

FIG. 36 is a diagram illustrating a modification of the radiographic imaging apparatus 100-10 according to the tenth exemplary embodiment. On the front surface 4001 illustrated in FIG. 36, the line-shaped indicator 4012a indicating the imaging area is located along the four sides, close to the end portions (side surfaces 4002) of the housing 4101. This indicator 4012a may be configured as a non-low-friction region to serve as an anti-slip measure when the user grips the radiographic imaging apparatus 100-10.

Low-friction regions given low-friction treatment typically have low wettability and tend to have poor adhesion with other materials. It is therefore difficult to first apply low-friction treatment and then form the non-low-friction regions thereon. Accordingly, if low-friction and non-low-friction regions are to coexist on the housing 4101, the regions with different coefficients of kinetic friction can be formed by locally forming areas where low-friction treatment is not applied.

### Eleventh Exemplary Embodiment

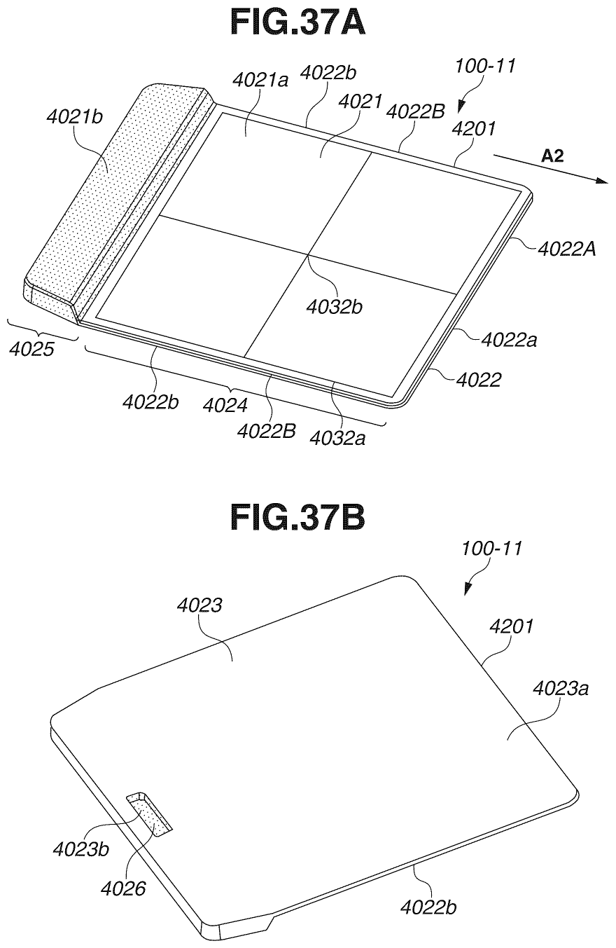

A radiographic imaging apparatus 100-11 according to an eleventh exemplary embodiment will be described with reference to FIGS. 37A and 37B. In the following description, a description of items common to the tenth exemplary embodiment is omitted, and differences from the tenth exemplary embodiment will mainly be described. FIGS. 37A and 37B are diagrams illustrating an example of the appearance of the radiographic imaging apparatus 100-11 according to the eleventh exemplary embodiment. Specifically, FIG. 37A is a perspective view as seen from the front side. FIG. 37B is a perspective view as seen from the rear side. The radiographic imaging apparatus 100-11 includes a housing 4201 of low-profile box shape that constitutes an outer casing thereof. The housing 4201 includes a front surface 4021 that constitutes an incident surface of radiation such as X-rays, a rear surface 4023 that is opposed to the front surface 4021, and side surfaces 4022 that connect the front surface 4021 and the rear surface 4023. The housing 4201 includes a thin section 4024 and a thick section 4025 one step higher than the thin section 4024 on the front surface 4021 side. An imaging area corresponding to a radiation detection panel 4009 is located in the thin section 4024. A line-shaped indicator 4032a indicating the imaging area and a line-shaped indicator 4032b indicating the center of the imaging area are disposed on the front surface 4021. Like the housing 4101 of the radiographic imaging apparatus 100-10 according to the tenth exemplary embodiment, the housing 4201 may be configured by combining a front cover, a rear cover, and a frame, or some of which may be integrated, for example. The thin section 4024 and the thick section 4025 may be integrated, or configured as separate components.

In such a radiographic imaging apparatus 100-11, the thickness of the thin section 4024 can be reduced, for example, by accommodating a not-illustrated battery and control substrate in the thick section 4025 located at one end of the housing 4201. Conventionally, radiographic imaging apparatuses have often been provided in sizes compliant with International Organization for Standardization (ISO) 4090:2001, with a thickness of approximately 15 mm to 16 mm. By contrast, according to the present exemplary embodiment, the thin section 4024 has a thickness of 10.0 mm or less, specifically 8.0 mm or so. Since the radiographic imaging apparatus 100-11 with the thin section 4024 of such a small thickness is inserted and removed into/from under the subject such as a patient lying on the bed, the step created by the thickness of the radiographic imaging apparatus 100-11 is small, and the reaction force occurring between the end portion of the radiographic imaging apparatus 100-11 and the subject can be alleviated. Since the radiographic imaging apparatus 100-11 has a fixed insertion direction (arrow A2 in FIG. 37A), a side surface 4022A serving as the insertion end, i.e., the side surface 4022A opposed to the thick section 4025 among the side surfaces 4022 connected to the thin section 4024 may be provided with sloped portions, curved portions, chamfers, etc. This makes the radiographic imaging apparatus 100-11 even easier to insert into under the subject such as a patient lying on the bed.

Here, a low-friction region 4021a given low-friction treatment is disposed on the front surface 4021 of the housing 4201. In the example illustrated in FIG. 37A, the thick section 4025 is configured as a non-low-friction region 4021b, and the other area the low-friction region 4021a. In FIG. 37A, the non-low-friction region 4021b is illustrated with a dot pattern, which is applied for the sake of convenience in illustration. Of the front surface 4021, the thin section 4024 is the part that contacts the subject such as a patient when the radiographic imaging apparatus 100-11 is placed under the subject such as a patient lying on the bed. The provision of the low-friction region 4021a here facilitates the movement of the radiographic imaging apparatus 100-11 in inserting, removing, and positioning the radiographic imaging apparatus 100-11. The low-friction region 4021a is desirably disposed over a wide area of the thin section 4024, and particularly over the imaging area corresponding to the radiation detection panel 4009 for easier movement.

Moreover, a low-friction region 4023a given low-friction treatment is disposed on the rear surface 4023 of the housing 4201. In the example illustrated in FIG. 37B, a grip portion 4026 that is a recess is disposed in the rear surface 4023, at a position behind the thick section 4025. The grip portion 4026 is configured as a non-low-friction region 4023b, and the other area the low-friction region 4023a. In FIG. 37B, the non-low-friction region 4023b is illustrated with a dot pattern, which is applied for the sake of convenience in illustration. The rear surface 4023 is the part that contacts the bed sheet when placed under the subject such as a patient lying on the bed. The provision of the low-friction region 4023a here facilitates the movement of the radiographic imaging apparatus 100-11 in inserting, removing, and positioning the radiographic imaging apparatus 100-11. The low-friction region 4023a is desirably disposed over a wide area of the rear surface 4023.

The user pinches and holds the housing 4201 with the thumb in the grip portion 4026 of the rear surface 4023 and the other fingers in contact with the thick section 4025, for example. Alternatively, the user pinches and holds the housing 4201 with the fingers other than the thumb in the grip portion 4026 of the rear surface 4023 and the thumb in contact with the thick section 4025, for example. The grip portion 4026 to thus place the thumb or fingers in is desirably configured as a high-friction region given high-friction treatment, and suitably has a coefficient of kinetic friction of 0.50 or more. Moreover, configuring the thick section 4025 as the non-low-friction region 4021b, or preferably a high-friction region with a coefficient of kinetic friction of 0.50 or more, enables a firm grip.

Furthermore, a low-friction region 4022a given low-friction treatment is disposed on the side surface 4022A serving as the insertion end among the side surfaces 4022 of the housing 4201. The provision of the low-friction region 4022a here can reduce catching on the patient gown, sheet, and the like when the radiographic imaging apparatus 100-11 is moved. Meanwhile, non-low-friction regions 4022b are dispose on left and right side surfaces 4022B orthogonal to the side surface 4022A. The side surfaces 4022B are considered to be less likely to contact the subject such as a patient, and the provision of the non-low-friction regions 4022b here can thus improve the ease of holding. The low-friction region 4022a and the non-low-friction regions 4022b of the side surface 4022 are just an example. For example, an area including the side surface 4022A and part of both side surfaces 4022B may be configured as a low-friction region 4022a, and the remaining areas of the side surfaces 4022B may be configured as non-low-friction regions 4022b, etc.

As described above, the low-friction regions 4021a and 4023a with a coefficient of kinetic friction of 0.15 or less are disposed on the front surface 4021 and the rear surface 4023 of the housing 4201. This can reduce the force needed to move the radiographic imaging apparatus 100-11 under the subject such as a patient lying on the bed. Moreover, the subject such as a patient is less likely to experience pain from abrasion when the radiographic imaging apparatus 100-11 is moved. The radiographic imaging apparatus 100-11 that can improve workability and reduce burden on the subject such as a patient can thus be provided.

The low-friction regions described in FIGS. 37A and 37B are just an example and not restrictive. At least either of, and preferably both of, the front surface 4021 and the rear surface 4023 can be provided with low-friction regions.

While the preferred tenth and eleventh exemplary embodiments of the present invention have been described above, these exemplary embodiments are not restrictive, and various modifications and changes can be made without departing from the gist thereof. Moreover, the foregoing exemplary embodiments may be combined as appropriate.

The tenth and eleventh exemplary embodiments of the present invention include the features described in the following supplementary notes.

### [Supplementary Note 50]

A radiographic imaging apparatus including:
a housing including a front surface constituting an incident surface of radiation, a rear surface opposed to the front surface, and side surfaces connecting the front surface and the rear surface; and
a radiation detection panel accommodated in the housing,
wherein a low-friction region with a coefficient of kinetic friction of 0.15 or less is disposed on at least either of the front surface and the rear surface of the housing.

### [Supplementary Note 51]

The radiographic imaging apparatus according to supplementary note 50, wherein the low-friction region is disposed on the front surface and the rear surface of the housing.

### [Supplementary Note 52]

The radiographic imaging apparatus according to supplementary note 50 or 51, wherein the low-friction region is disposed on an imaging area of the front surface.

### [Supplementary Note 53]

The radiographic imaging apparatus according to any one of supplementary notes 50 to 52, wherein the low-friction region with a coefficient of kinetic friction of 0.15 or less is disposed on the side surfaces of the housing.

### [Supplementary Note 54]

The radiographic imaging apparatus according to any one of supplementary notes 50 to 53, wherein the low-friction region is formed of paint or ink applied to the housing.

### [Supplementary Note 55]

The radiographic imaging apparatus according to supplementary note 54, wherein the paint or ink is a urethane-based or acrylic urethane-based paint or ink.

### [Supplementary Note 56]

The radiographic imaging apparatus according to supplementary note 54 or 55, wherein the paint or ink is compounded with fine particles.

### [Supplementary Note 57]

The radiographic imaging apparatus according to any one of supplementary notes 54 to 56, wherein the paint or ink is compounded with a material having an antibacterial effect.

### [Supplementary Note 58]

The radiographic imaging apparatus according to any one of supplementary notes 50 to 57, wherein the low-friction region and a non-low-friction region with a coefficient of kinetic friction higher than that of the low-friction region are disposed on at least either of the front surface and the rear surface of the housing.

### [Supplementary Note 59]

The radiographic imaging apparatus according to supplementary note 51, wherein the low-friction region and a non-low-friction region with a coefficient of kinetic fiction higher than that that of the low-friction region are disposed on the front surface and the rear surface of the housing.

### [Supplementary Note 60]

The radiographic imaging apparatus according to supplementary note 59,
wherein a grip portion is disposed in the rear surface of the housing, the grip portion being a recess located along a side,
wherein the grip portion is configured as the non-low-friction region, and
wherein a predetermined area of the front surface of the housing is configured as the non-low-friction region, the predetermined area corresponding to the grip portion.

### [Supplementary Note 61]

The radiographic imaging apparatus according to any one of supplementary notes 58 to 60, wherein a high-friction region with a coefficient of kinetic friction of 0.50 or more is disposed as the non-low-friction region.

### [Supplementary Note 62]

The radiographic imaging apparatus according to any one of supplementary notes 50 to 61, wherein the housing includes a thin section and a thick section one step higher than the thin section on the front surface side, and the low-friction portion is disposed on the thin section of the front surface.

### [Supplementary Note 63]

The radiographic imaging apparatus according to supplementary note 62, wherein a/the non-low-friction region with a/the coefficient of kinetic friction higher than that of the low-friction portion is disposed on the thick section.

### [Supplementary Note 64]

The radiographic imaging apparatus according to supplementary note 62 or 63, wherein a sloped portion or a curved portion is formed on a side surface opposed to the thick section among the side surfaces connected to the thin section, and the low-friction region is disposed on the side surface.

### [Supplementary Note 65]

The radiographic imaging apparatus according to any one of supplementary notes 62 to 64, wherein a/the non-low-friction region is disposed on a side surface orthogonal to a/the side surface opposed to the thick section among the side surfaces connected to the thin section.

According to the features described in the foregoing supplementary notes 50 to 65, a radiographic imaging apparatus that can improve workability and reduce burden on the subject can be provided.

### Twelfth Exemplary Embodiment

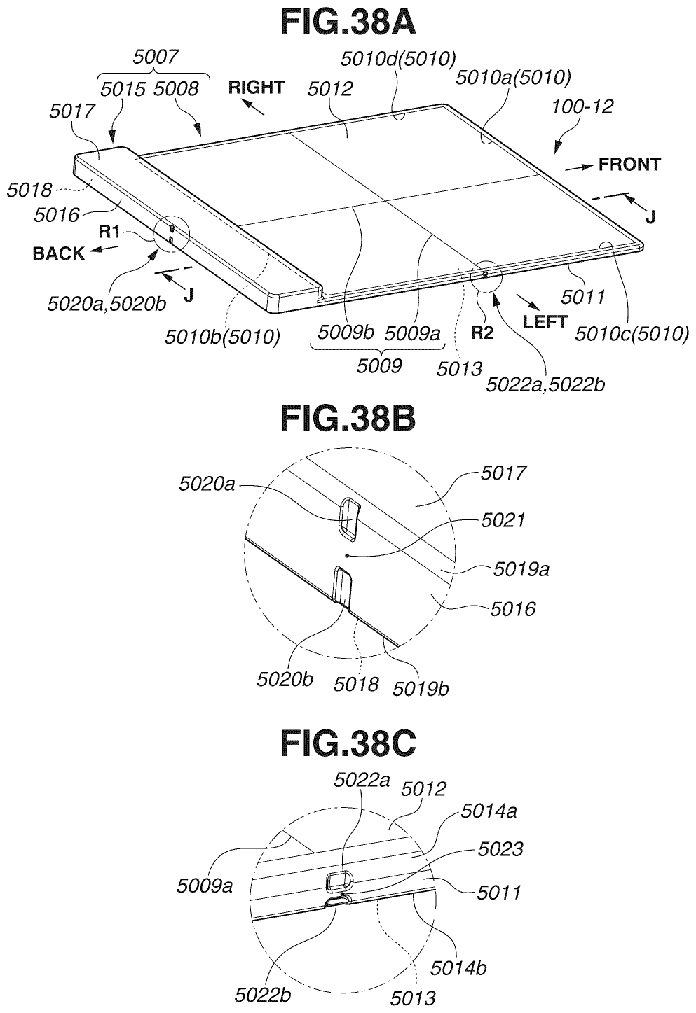

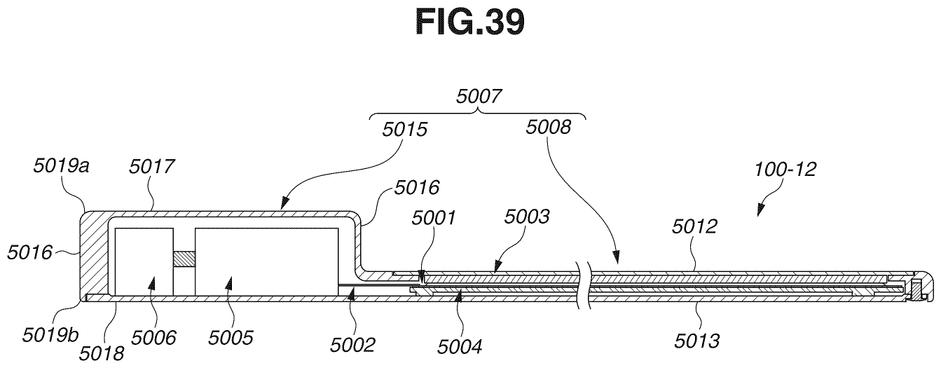

FIGS. 38A to 38C are diagrams illustrating an example of the appearance of a radiographic imaging apparatus 100-12 according to a twelfth exemplary embodiment. Specifically, FIGS. 38A to 38C are perspective views illustrating a configuration of the radiographic imaging apparatus 100-12. FIG. 39 is a sectional view of the radiographic imaging apparatus 100-12, and specifically a sectional view taken along line J-J illustrated in FIG. 38A. The radiographic imaging apparatus 100-12 obtains a radiographic image by irradiating a subject with radiation from a radiation generation apparatus and detecting the radiation transmitted through the subject. The radiographic image obtained by the radiographic imaging apparatus 100-12 is transferred to outside, displayed on a monitor or the like, and thereby used for diagnosis etc.

The radiographic imaging apparatus 100-12 includes a radiation detection panel 5001, a control substrate 5005, a battery 5006, and a housing 5007. The radiation detection panel 5001 is of a so-called indirect conversion system, including a sensor substrate on which a large number of photoelectric conversion elements (sensors) are arranged, a phosphor layer (scintillator layer) that is located on the sensor substrate, and a phosphor protective film. The radiation detection panel 5001 includes some or all of the photoelectric conversion elements in its effective imaging area. Here, the effective imaging area is an area that is capable of radiographic imaging and where the radiographic image is actually generated. The effective imaging area according to the present exemplary embodiment is rectangular as seen in a radiation incident direction, but the shape is not limited thereto. The sensor substrate is formed of a material such as glass and flexible plastic, but the material is not limited thereto. The phosphor protective film protects the phosphor. The phosphor protective film is formed of a material with low moisture permeability to the phosphor.

The radiation detection panel 5001 is not limited to the indirect conversion system, and may be of a direct conversion system. The radiation detection panel of the direct conversion system includes a conversion element unit where conversion elements formed of a-Se or the like and electrical elements such as TFTs are two-dimensionally arranged. The radiation detection panel 5001 is not limited to the indirect conversion system or the direct conversion system, either.

The control substrate 5005 functions as a control unit that controls the radiation detection panel 5001. The control substrate 5005 reads detection signals from the radiation detection panel 5001, and processes the read detection signals. The control substrate 5005 is connected to the radiation detection panel 5001 via a flexible circuit board 5002. The battery 5006 supplies necessary power to the radiographic imaging apparatus 100-12. Examples of the battery 5006 include a lithium-ion battery, an electric double layer capacitor, and an all-solid-state battery.

The housing 5007 functions as an external casing that includes (accommodates) the radiation detection panel 5001. To achieve portability and strength in a compatible manner, the housing 5007 is formed of a magnesium alloy, an aluminum alloy, fiber-reinforced plastic, plastic, or the like. The material is not limited thereto. The housing 5007 includes a thin section 5008 and a thick section 5015.

When imaging a subject such as a patient, the user inserts the radiographic imaging apparatus 100-12 into between the subject such as a patient and a bed or the like, and places the radiographic imaging apparatus 100-12 directly under the imaging site of the subject such as a patient. The arrow “front” in FIG. 38A represents the insertion direction in inserting the radiographic imaging apparatus 100-12 into between the subject such as a patient and the bed or the like, and the radiographic imaging apparatus 100-12 is inserted with the thin section 5008 ahead. Here, as illustrated in FIG. 38A, the thin section 5008 side of the housing 5007 will be referred to as the front, the thick section 5015 side will be referred to as the back, one side in a direction orthogonal to the front-to-back direction will be referred to as the right, and the other side will be referred to as the left.

The thin section 5008 is a section of which the thickness along the radiation incident direction is less than that of the thick section 5015. The thin section 5008 is rectangular as seen in the radiation incident direction. The thin section 5008 includes side surfaces 5011, an incident surface 5012, and a bottom surface 5013. The thin section 5008 accommodates the radiation detection panel 5001. In other words, the thin section 5008 overlaps the effective imaging area of the radiation detection panel 5001 in the radiation incident direction. A cushioning material 5003 is disposed between the incident surface 5012 of the thin section 5008 and the radiation detection panel 5001. The cushioning material 5003 protects the radiation detection panel 5001 from external force and the like. A support base 5004 is disposed between the bottom surface 5013 of the thin section 5008 and the radiation detection panel 5001. The support base 5004 supports the radiation detection panel 5001. The incident surface 5012 of the thin section 5008 is formed of a carbon fiber-reinforced plastic or the like with high radiation transmittance and excellent lightweight properties. The cushioning material 5003 is formed of a foamed resin, gel, or the like.

An indicator 5009 for indicating the center position of the effective imaging area and an indicator 5010 for indicating the contour of the effective imaging area are disposed on the incident surface 5012 of the thin section 5008. The indicators 5009 and 5010 may be implemented by painting or printing, by forming physical steps, or by changing the surface properties through formation of a texture pattern or the like or attachment of separate members. The indicators 5009 and 5010 may be directly provided on the incident surface 5012 of the thin section 5008, may be provided by attaching a sheet that is a separate member, or may be provided by attaching a painted or printed sheet.

The indicator 5009 according to the present exemplary embodiment includes two straight lines intersecting at right angles. Specifically, the indicator 5009 has a cross shape including a straight center line 5009a along the lateral direction and a straight center line 5009b along the front-to-back direction, and the center of the cross indicates the center position of the effective imaging area. Note that the indicator 5009 is not limited to the cross shape, as long as the center position of the effective imaging area can be recognized.

Meanwhile, the indicator 5010 according to the present exemplary embodiment includes a plurality of straight lines. Specifically, the indicator 5010 has a rectangular shape including straight contour lines 5010a and 5010b that are located apart from each other in the front-to-back direction and extend along the lateral direction, and straight contour lines 5010c and 5010b that are located apart from each other in the lateral direction and extend along the front-to-back direction. The contour lines 5010a and 5010b and the contour lines 5010c and 5010d intersect at right angles. The inside of the rectangle formed by the contour lines 5010a to 5010d is the effective imaging area. The indicator 5010 is not limited to the rectangular shape, as long as the effective imaging area can be recognized.

The thick section 5015 is a section of which the thickness along the radiation incident direction is greater than that of the thin section 5008. The thick section 5015 is rectangular as seen in the radiation incident direction. The thick section 5015 includes side surfaces 5016, a top surface 5017, and a bottom surface 5018. The thick section 5015 is located to adjoin the thin section 5008. Specifically, the thick section 5015 is located along one of the four sides of the rectangular shape of the thin section 5008, and has an elongated shape long along the one side. The thick section 5015 accommodates the control substrate 5005 and the battery 5006. In other words, the thick section 5015 overlaps the control substrate 5005 and the battery 5006 in the radiation incident direction.

In imaging a subject such as a patient, the radiographic imaging apparatus 100-12 is placed directly under the imaging site of the subject such as a patient. In doing so, a step created by the thickness of the radiographic imaging apparatus 100-12 comes into contact with the subject such as a patient to cause a reaction force, and the subject may feel discomfort. Conventional constant-thickness radiographic imaging apparatuses have often had sizes compliant with International Organization for Standardization (ISO) 4090:2001, with a thickness of approximately 15 mm to 16 mm. The housing 5007 according to the present exemplary embodiment includes the thin section 5008 thinner than the thick section 5015, which can reduce the step of the radiographic imaging apparatus 100-12. More specifically, by placing the thin section 5008 of the radiographic imaging apparatus 100-12 directly under the imaging site of the subject such as a patient, the reaction force occurring between the subject such as a patient and the end portion of the radiographic imaging apparatus 100-12 can be reduced, and the burden on the subject such as a patient can be reduced.

Specifically, to reduce the reaction force and maintain the layer configuration and mechanical strength in a compatible manner, the thin section 5008 has a thickness of approximately 8 mm (±1 mm). Note that the thickness of the thin section 5008 is not limited in particular, and is preferably 10.0 mm or less, more preferably 8.0 mm or less, to reduce the burden on the subject such as a patient. To maintain the layer configuration and mechanical strength, the thickness of the thin section 5008 is desirably 5.0 mm or more.

When the thin section 5008 of the radiographic imaging apparatus 100-12 is placed directly under the imaging site of the subject such as a patient, the indicators 5009 and 5010 disposed on the incident surface 5012 of the thin section 5008 are hidden by the back or other parts of the subject such as a patient and difficult to recognize visually or tactilely. In the present exemplary embodiment, as illustrated in FIGS. 38A to 38C, the housing 5007 includes recognition portions 5020a and 5020b located on the extension of the center line 5009b of the indicator 5009 indicating the center position of the effective imaging area. Even when the radiographic imaging apparatus 100-12 is placed behind the back or other parts of the subject such as a patient and the incident surface 5012 is hidden, the center position of the effective imaging area of the radiographic imaging apparatus 100-12 can thus be recognized by visually observing or touching the recognition portions 5020a and 5020b.

A configuration of the recognition portions 5020a and 5020b will be described with reference to FIG. 38B. FIG. 38B is an enlarged perspective view of part R1 in FIG. 38A.

The recognition portions 5020a and 5020b according to the present exemplary embodiment are disposed in the side surface 5016 on the back side of the thick section 5015, on the extension of the center line 5009b of the indicator 5009. The recognition portions 5020a and 5020b are located apart from each other in the radiation incident direction. The recognition portions 5020a and 5020b according to the present exemplary embodiment are implemented by steps. Specifically, the recognition portions 5020a and 5020b have a groove shape recessed from the side surface 5016. The thick section 5015 according to the present exemplary embodiment includes a sloped surface 5019a formed at the border between the side surface 5016 and the top surface 5017, and a sloped surface 5019b formed at the border between the side surface 5016 and the bottom surface 5018. The recognition portions 5020a and 5020b are formed beyond the side surface 5016 to reach the sloped surfaces 5019a and 5019b, respectively. A sliding portion 5021 is formed between the recognition portions 5020a and 5020b. The sliding portion 5021 is the same surface as the side surface 5016. The sliding portion 5021 can reduce the possibility of catching of the recognition portions 5020a and 5020b when the radiographic imaging apparatus 100-12 is slid over a bed, table, charging cradle, or the like along the side surface 5016. The sloped surfaces 5019a and 5019b according to the present exemplary embodiment have a fillet shape (curved chamfers), but may have a flat shape (flat chamfers).

In the present exemplary embodiment, the thin section 5008 is also provided with recognition portions 5022a and 5022b. A configuration of the recognition portions 5022a and 5022b will be described with reference to FIG. 38C. FIG. 38C is an enlarged perspective view of part R2 in FIG. 38A.

The recognition portions 5022a and 5022b according to the present exemplary embodiment are disposed in the left side surface 5011 of the thin section 5008, on the extension of the center line 5009a of the indicator 5009. The recognition portions 5022a and 5022b are located apart from each other in the radiation incident direction. The recognition portions 5022a and 5022b according to the present exemplary embodiment are implemented by steps. Specifically, the recognition portions 5022a and 5022b have a groove shape recessed from the side surface 5011. The thin section 5008 according to the present exemplary embodiment includes a sloped surface 5014a formed at the border between the side surface 5011 and the incident surface 5012, and a sloped surface 5014b formed at the border between the side surface 5011 and the bottom surface 5013. The recognition portions 5022a and 5022b are formed beyond the side surface 5011 to reach the sloped surfaces 5014a and 5014b, respectively. A sliding portion 5023 is formed between the recognition portions 5022a and 5022b. The sliding portion 5023 is the same surface as the side surface 5011, and has a function similar to that of the foregoing sliding portion 5021. The recognition portions 5022a and 5022b are also disposed in the right side surface 5011 of the thin section 5008, on the extension of the center line 5009a of the indicator 5009. The recognition portions 5022a and 5022b may also be disposed in the front side surface 5011 of the thin section 5008 on the extension of the center line 5009a of the indicator 5009. The sloped surfaces 5014a and 5014b according to the present exemplary embodiment have a fillet shape (curved chamfers), but may have a flat shape (flat chamfers).

The recognition portions 5020a and 5020b according to the present exemplary embodiment may be disposed in at least one of the side surface 5016 on the back side of the thick section 5015, the top surface 5017, the bottom surface 5018, and the sloped surfaces 5019a and 5019b, on the extensions of the contour lines 5010c and 5010d of the indicator 5010. The recognition portions 5020a and 5020b according to the present exemplary embodiment may also be disposed in the border portion between the thin section 5008 and the thick section 5015 (for example, in the side surface 5016 on the front side of the thick section 5015). Note that the recognition portions disposed in the border portion between the thin section 5008 and the thick section 5015 can be hidden behind the subject such as a patient or difficult to access due to the thick section 5015. By contrast, the provision of the recognition portions 5020a and 5020b in the side surface 5016 on the back side of the thick section 5015 as described above can improve recognizability in various situations.

### Modification 1 of Twelfth Exemplary Embodiment

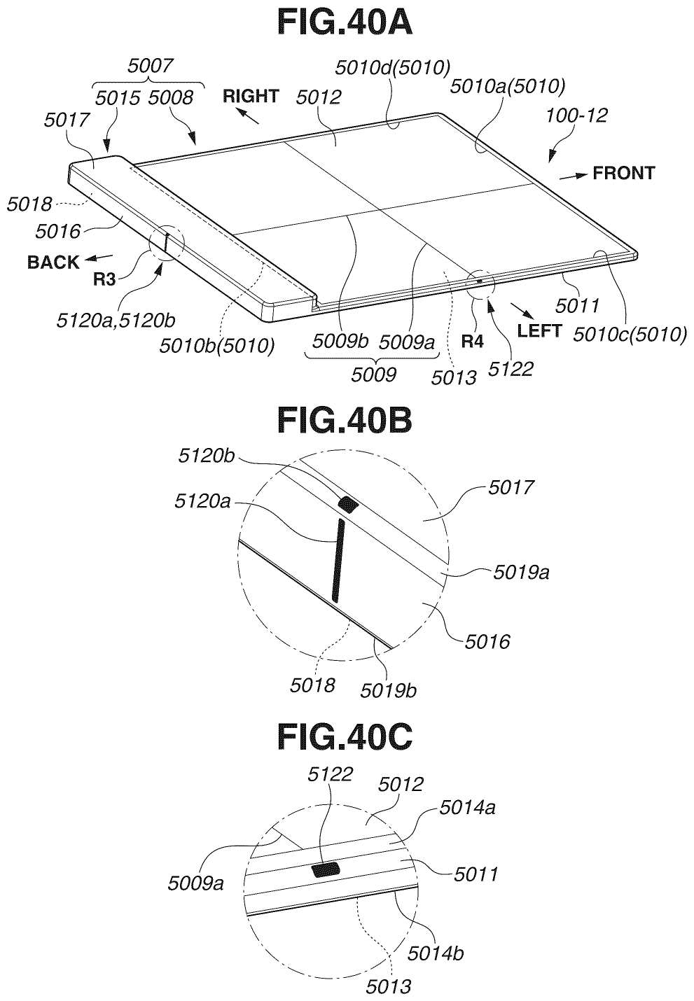

FIGS. 40A to 40C are diagrams illustrating modification 1 of the configuration of the radiographic imaging apparatus 100-12 according to the twelfth exemplary embodiment. In FIGS. 40A to 40C, components similar to those of FIGS. 38A to 38C and 39 are denoted by the same reference numerals, and a description thereof will be omitted. The housing 5007 of the radiographic imaging apparatus 100-12 according to modification 1 illustrated in FIGS. 40A to 40C includes recognition portions 5120a, 5120b, and 5122.

A configuration of the recognition portions 5120a and 5120b will be described with reference to FIG. 40B. FIG. 40B is an enlarged perspective view of part R3 in FIG. 40A. The recognition portions 5120a and 5120b according to this modification are disposed in the side surface 5016 on the back side of the thick section 5015 and the sloped surface 5019a, on the extension of the center line 5009b of the indicator 5009. The recognition portions 5120a and 5120b according to this modification are implemented using light sources such as LEDs. The center position of the effective imaging area of the radiographic imaging apparatus 100-12 can therefore be visually recognized. The provision of the recognition portion 5120a in the wide side surface 5016 can improve visibility since the recognition portion 5102a itself can be increased in size. Meanwhile, the provision of the recognition portion 5102b in the sloped surface 5019a enables visual recognition of the recognition portion 5102b from both the incident surface 5012 side and the side surface 5016 side of the housing 5007.

A configuration of the recognition portion 5122 will be described with reference to FIG. 40C. FIG. 40C is an enlarged perspective view of part R4 in FIG. 40A. The recognition portion 5122 according to this modification is disposed in the left sloped surface 5014a of the thin section 5008, on the extension of the center line 5009a of the indicator 5009. The recognition portion 5122 according to this modification is implemented using a light source such as an LED. The center position of the effective imaging area of the radiographic imaging apparatus 100-12 can therefore be visually recognized. Similarly, the recognition portion 5122 is also disposed in the right sloped surface 5014a of the thin section 5008, on the extension of the center line 5009a of the indicator 5009. The recognition portion 5122 may also be disposed in the sloped surface 5014a on the front side of the thin section 5008, on the extension of the center line 5009b of the indicator 5009.

The color of the recognition portions 5120a, 5120b, and 5122 may be changed depending on the model of the radiographic imaging apparatus. Changing the color of the recognition portions 5120a, 5120b, and 5122 enables model distinction if multiple models of radiographic imaging apparatuses with the same outer shape are owned. Moreover, the color of the recognition portions 5120a, 5120b, and 5122 may be changed based on the state of the model, thereby providing a status indicator function as well. The recognition portions 5120a, 5120b, and 5122 may be implemented by applying color different from that of the surroundings of the recognition portions 5120a, 5120b, and 5122, instead of using light sources such as LEDs. The recognition portions 5120a, 5120b, and 5122 may be implemented by changing the surface properties (changing the surface friction) of housing 5007 through the formation of a texture pattern and the like or the attachment of separate members.

### Modification 2 of Twelfth Exemplary Embodiment

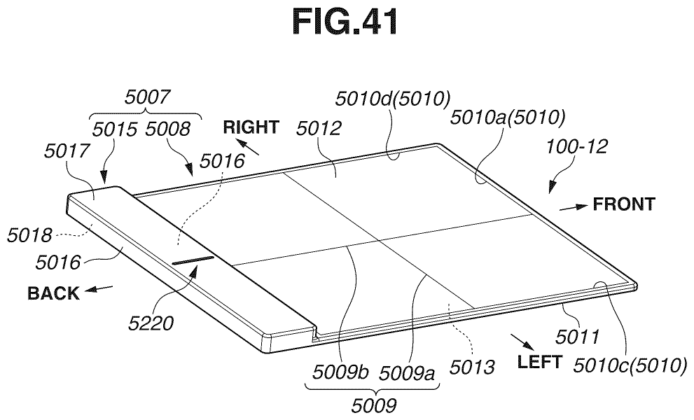

FIG. 41 is a diagram illustrating modification 2 of the configuration of the radiographic imaging apparatus 100-12 according to the twelfth exemplary embodiment. In FIG. 41, components similar to those of FIGS. 38A to 38C and 39 are denoted by the same reference numerals, and a description thereof will be omitted. The housing 5007 of the radiographic imaging apparatus 100-12 according to modification 2 illustrated in FIG. 41 includes a recognition portion 5220.

The recognition portion 5220 according to this modification is disposed in the top surface 5017 of the thick section 5015, on the extension of the center line 5009b of the indicator 5009. The recognition portion 5220 according to this modification is implemented by a step. Specifically, the recognition portion 5220 has a groove shape recessed from the top surface 5017. The recognition portion 5220 has a straight shape along the extension of the center line 5009b of the indicator 5009. The recognition portion 5220 is formed in the top surface 5017 from a position close to the side surface 5016 on the back side to a position close to the side surface 5016 on the front side of the thick section 5015.

Since the radiographic imaging apparatus has the thick section 5015, the outer shape of the housing 5007 is greater than the effective imaging area, and the recognition portion and the effective imaging area can be far apart if the recognition portion is located on the side surface 5016 on the back side of the thick section 5015. On the other hand, if the recognition portion is located at the border between the thick section 51015 and the thin section 5008, close to the effective imaging area, the recognition portion is difficult to access. The provision of the recognition portion 5220 in the top surface 5017 of the thick section 5015 as in this modification can prevent the recognition portion 5220 and the effective imaging area from being far apart. Moreover, since the top surface 5017 of the thick section 5015 is closer to the radiation generation apparatus than the incident surface 5012 of the thin section 5008 in the radiation incident direction, the user can recognize the recognition portion 5220 disposed in the top surface 5107 more easily. Furthermore, the top surface 5017 of the thick section 5015 is less likely to be hidden under the subject than the thin section 5008, the user can better recognize the recognition portion 5220 disposed in the top surface 5017.

The recognition portion 5220 is not limited to the step, and may be implemented using a light source or implemented by applying color different from that of the surroundings of the recognition portion 5220. The recognition portion 5220 may be implemented by changing the surface properties (changing the surface friction) of the housing 5007 through the formation of a texture pattern and the like or the attachment of separate members.

In radiographic imaging apparatuses of which the effective imaging area is implemented by the thin section 5008 and that are easy to handle like the present exemplary embodiment, recognition portions can be provided on the thin section 5008 close to the effective imaging area or on the border portion between the thick section 5015 and the thin section 5008. However, the thin section 5008 and the border portion are areas that are likely to be hidden when the radiographic imaging apparatus is placed directly under the imaging site of the subject such as a patient. In addition, the border portion is difficult to access due to the thick section 5015. By contrast, the provision of the recognition portion for enabling recognition of the effective imaging area on the thick section 5015 as in the present exemplary embodiment can facilitate the positioning of the effective imaging area since the recognition portion is highly accessible and visible.

### Thirteenth Exemplary Embodiment

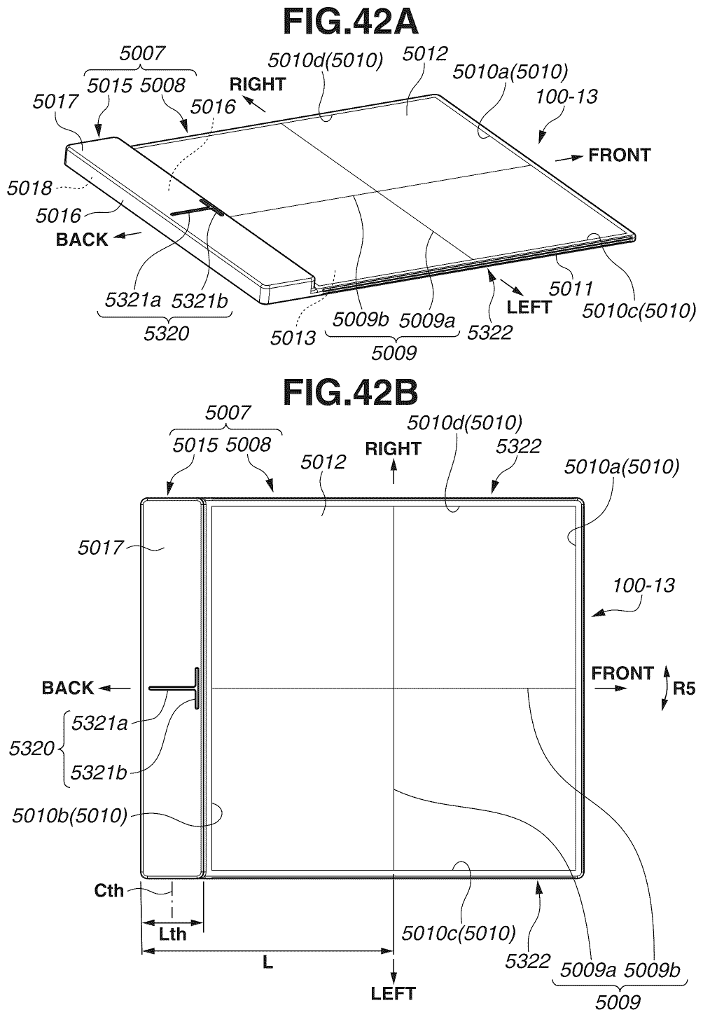

FIGS. 42A and 42B are diagrams illustrating an example of the appearance of a radiographic imaging apparatus 100-13 according to a thirteenth exemplary embodiment. Specifically, FIG. 42A is a perspective view illustrating a configuration of the radiographic imaging apparatus 100-13. FIG. 42B is a plan view of the radiographic imaging apparatus 100-13 seen in a radiation incident direction. Components similar to those of the twelfth exemplary embodiment are denoted by the same reference numerals, and a description thereof will be omitted.

A housing 5007 of the radiographic imaging apparatus 100-13 according to the present exemplary embodiment includes a recognition portion 5320 disposed on a top surface 5017 of a thick section 5015. The recognition portion 5320 is implemented by a step. Specifically, the recognition portion 5320 has a protruding shape protruding from the top surface 5017 to a radiation generation apparatus side. The recognition portion 5320 includes two straight line portions 5321a and 5321b. In a plan view, the straight line portions 5321a and 5321 intersect at right angles. The recognition portion 5320 is formed in a T-shape with the straight line portions 5321a and 5321b.

The straight line portion 5321a is parallel to the center line 5009b of the effective imaging area or contour lines 5010c and 5010d. The straight line portion 5321b is parallel to the center line 5009a of the effective imaging area or contour lines 5010a and 5010b. The straight line portion 5331a is located on the extension of the center line 5009b of the indicator 5009 and has a straight shape along the extension of the center line 5009b. In other words, the straight line portion 5321a is parallel to the center line 5009b. The straight line portion 5321a is also parallel to contour lines 5010c and 5010d of an indicator 5010. The straight line portion 5321a is formed on the top surface 5017 from a position close to the side surface 5016 on the back side to a position close to the side surface 5016 on the front side. The straight line portion 5321a intersects the straight line portion 5321b at its front-side position. The straight line portion 5321b is located on the extension of the center line 5009b of the indicator 5009 and has a straight shape orthogonal to the center line 5009b. In other words, the straight line portion 5321b is parallel to the center line 5009a. The straight line portion 5321b is also parallel to contour lines 5010a and 5010b of the indicator 5010. The straight line portion 5321b is formed on the top surface 5017 of the thick section 5015, at a position offset toward the thin section 5008. Specifically, with the length of the thick section 5015 in the front-to-back direction as Lth and the center of the length Lth as Cth, the straight line portion 5321b is located on the thin section 5008 side of the center Cth.

In the radiographic imaging apparatus 100-13 where the thin section 5008 constitutes the effective imaging area, the outer shape of the housing 5007 is larger on the thick section 5015 side. More specifically, as illustrated in FIG. 42B, the length L from the center position of the effective imaging area to the outer shape of the thick section 5015 is greater. When the user places the radiographic imaging apparatus 100-13 directly under the subject such as a patient while gripping the thick section 5015, the effective imaging area can deviate in the direction of the arrow R5 illustrated in FIG. 42B and be located at a position different from a desired position due to the large distance L. Here, the user may have difficulty in recognizing the subtle difference in angle based only on the straight line portion 5321a, and may touch the outer shape and the like of the housing 5007 that are parallel to the contour lines 5010a and 5010b of the indicator 5010 to check the position of the effective imaging area.

In the present exemplary embodiment, the recognition portion 5320 including the two orthogonal straight line portions 5321a and 5321b are disposed on the top surface 5017 of the thick section 5015. The user can therefore not only recognize the center position of the effective imaging area by visually observing or touching the recognition portion 5320, but also easily check whether the angle deviates in the direction of the arrow R5 based on the positions of the two orthogonal straight line portions 5321a and 5321b. This eliminates the need for the user to perform operations such as touching the outer shape of the housing 5007 parallel to the contour lines 5010a and 5010b of the indicator 5010, and can thus improve work efficiency related to radiographic imaging.

Since the straight line portion 5321b is offset toward the thin section 5008, the user can recognize the angular relationship between the subject such as a patient and the effective imaging area more easily. The border portion between the top surface 5017 and the side surface 5016 on the front side of the thick section 5015 is likely to contact the subject such as a patient, and is thus configured in a fillet shape (curved chamfer). Whether the border portion between the top surface 5017 and the front side surface 5016 of the thick section 5015 is parallel to the effective imaging area may therefore be difficult to recognize by visually observing the border portion. The angular relationship between the subject and the effective imaging area can be easily recognized by visually observing the recognition portion 5320 including the two orthogonal straight line portions 5321a and 5321b as in the present exemplary embodiment.

In the present exemplary embodiment, the thin section 5008 is also provided with recognition portions 5322.

The recognition portions 5322 according to the present exemplary embodiment are disposed on the side surfaces 5011 of the thin section 5008. The recognition portions 5322 are implemented by a step on an area where the contour of the effective imaging area is projected. Specifically, the recognition portions 5322 have a protruding shape protruding outward from the side surfaces 5011. The user can thus recognize the effective imaging area in a tactile manner.

While the recognition portion 5320 according to the present exemplary embodiment is described to be T-shaped, this is not restrictive. The recognition portion 5320 may be cross-shaped, I-shaped, or H-shaped. The straight line portions 5321a and 5321b are not limited to intersecting each other and may be separated. While the recognition portion 5320 according to the present exemplary embodiment is described to have a protruding shape protruding from the top surface 5017 to the radiation generation apparatus side, this is not restrictive. The recognition portion 5320 may have a groove shape recessed from the top surface 5017 in the radiation incident direction. The recognition portion 5320 of the protruding shape can reduce dust accumulation, and the recognition portion 5320 of the recessed shape can prevent catching on surrounding items. The recognition portion 5320 desirably has a shape easily recognizable with fingertips, which have a keen sense of touch. Note that the recognition portion 5320 is not limited to a step, and may be implemented using a light source or by applying color different from that of the surroundings of the recognition portion 5320. The recognition portion 5320 may be implemented to change the surface properties (change the surface friction) of the housing 5007 by forming a texture pattern or the like or by attaching a separate member.

### Modification 1 of Thirteenth Exemplary Embodiment

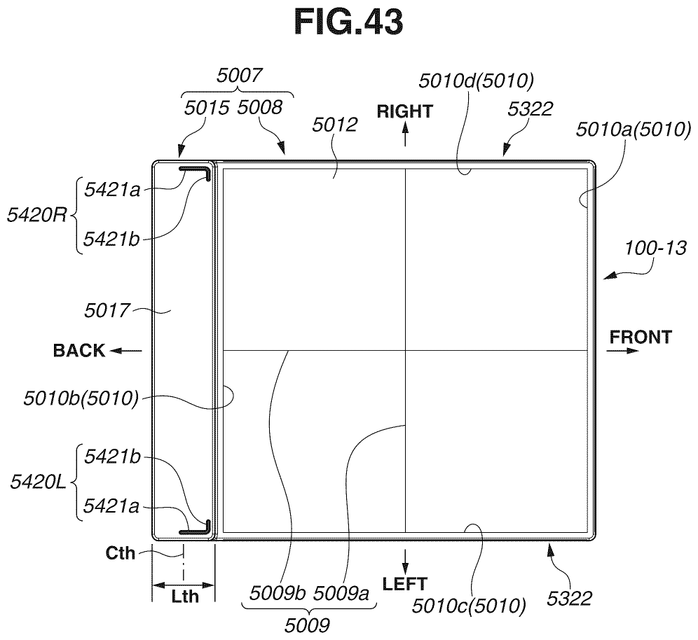

FIG. 43 is a diagram illustrating modification 1 of the configuration of the radiographic imaging apparatus 100-13 according to the thirteenth exemplary embodiment. In FIG. 43, components similar to those of FIGS. 42A and 42B are denoted by the same reference numerals, and a description thereof will be omitted. The housing 5007 of the radiographic imaging apparatus 100-13 according to modification 1 illustrated in FIG. 43 includes a plurality of (here, two) recognition portions 5420L and 5420R on the top surface 5017 of the thick section 5015. The recognition portions 5420L and 5420R are implemented by steps. Specifically, the recognition portions 5420L and 5420R have a protruding shape protruding from the top surface 5017 to the radiation generation apparatus side. The recognition portions 5420L and 5420R are located laterally apart from each other and laterally symmetrical. Here, the recognition portion 5420L will mainly be described. The recognition portion 5420L is formed in an L-shape with a straight line portion 5421a and a straight line portion 5421b.

The two straight line portions 5421a and 5421b are parallel to the center lines 5009a and 5009b of the effective imaging area or parallel to the contour lines 5010a to 5010d of the effective imaging area. The straight line portion 5421a is located on the extension of the contour line 5010c of the indicator 5010 and has a straight shape along the extension of the contour line 5010c. In other words, the straight line portion 5421a is parallel to the contour line 5010c. The straight line portion 5421a is also parallel to the center line 5009b of the indicator 5009 and the contour line 5010d of the indicator 5010. The straight line portion 5421a intersects the straight line portion 5421b at its front-side position. The straight line portion 5421b is located on the extension of the contour line 5010c of the indicator 5010 and has a straight shape orthogonal to the contour line 5010c. In other words, the straight line portion 5421b is parallel to the center line 5009a of the indicator 5009 and the contour lines 5010a and 5010b of the indicator 5010. The straight line portion 5421b is formed on the top surface 5017 of the thick section 5015, at a position offset toward the thin section 5008. Specifically, the straight line portion 5421b is located on the thin section 5008 side of the center Cth.

In this modification, the two recognition portions 5420R and 5420L are located on the top surface 5017 of the thick section 5015, laterally apart from each other. The user can recognize the contour of the effective imaging area and the center position of the effective imaging area by visually observing or touching the two recognition portions 5420R and 5420L disposed on the thick section 5015 that is highly accessible. The recognition portions 5420R and 5420L each have an L shape constituted by two straight line portions 5421a and 5421b intersecting at right angles. The user can thus easily check whether the angle deviates in the foregoing direction of the arrow R5 by visually observing or touching either of the recognition portions 5420R and 5420L.

While the two recognition portions 5420R and 5420L of this modification are described to be L-shaped, this is not restrictive. The recognition portions 5420R and 5420L may be cross-shaped, I-shaped, or H-shaped. The straight line portions 5421a and 5421b are not limited to intersecting each other, and may be separate from each other.

According to the present exemplary embodiment, the recognition portion including the two straight line portions intersecting at right angles is disposed on the thick section 5015. An angular deviation of the effective imaging area can thus be easily checked, and the effective imaging area can be easily positioned.

### Fourteenth Exemplary Embodiment

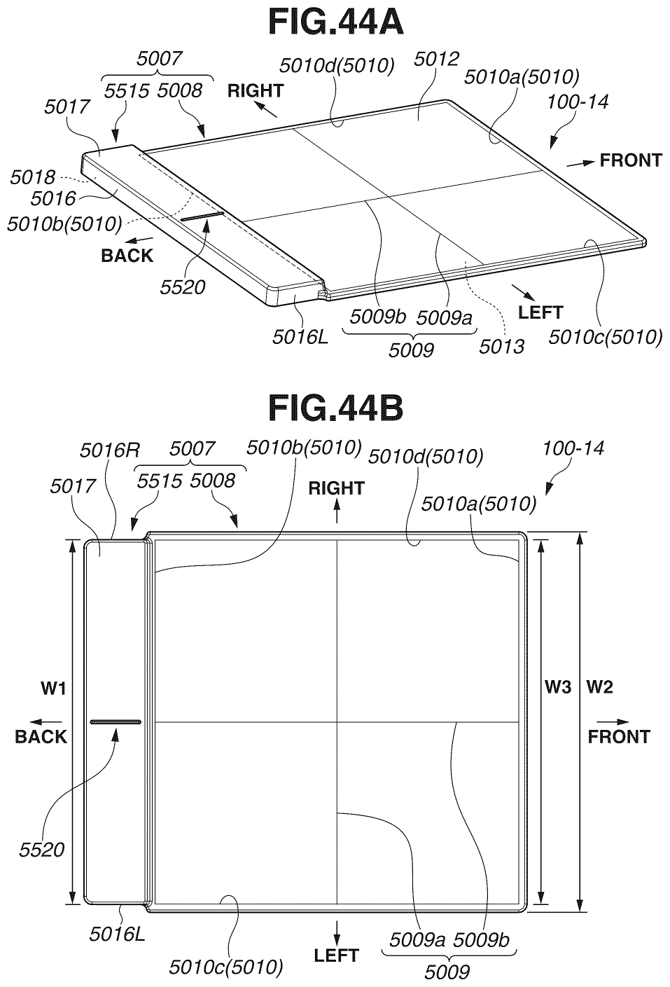

FIGS. 44A and 44B are diagrams illustrating an example of the appearance of a radiographic imaging apparatus 100-14 according to a fourteenth exemplary embodiment. Specifically, FIG. 44A is a perspective view illustrating a configuration of the radiographic imaging apparatus 100-14. FIG. 44B is a plan view of the radiographic imaging apparatus 100-14 seen in a radiation incident direction. Components similar to those of the twelfth exemplary embodiment are denoted by the same reference numerals, and a description thereof will be omitted. A housing 5007 according to the present exemplary embodiment includes a thin section 5008 and a thick section 5515. The thick section 5515 according to the present exemplary embodiment differs from the thick section 5015 according to the twelfth exemplary embodiment in size.

In the present exemplary embodiment, the thick section 5515 itself functions as a recognition portion for enabling the user to recognize the contour of the effective imaging area. Specifically, as illustrated in FIG. 44B, a width W1 of the thick section 5515 is configured to be less than a width W2 of the thin section 5008 and the same as a width W3 of the effective imaging area. Among side surfaces 5016 of the thick section 5515, the one on the left will be referred to as a left side surface 5016L, and the one on the right will be referred to as a right side surface 5016R. In the present exemplary embodiment, the left side surface 5016L of the thick section 5515 is located on the extension of the contour line 5010c of the indicator 5010. The right side surface 5016R of the thick section 5515 is located on the extension of the contour line 5010d of the indicator 5010. In such a manner, the thick section 5515 can indicate the effective imaging area with its outer shape.

Even when the thin section 5008 is covered by the subject such as a patient, the user can thus figure out the effective imaging area by touching the outer shape of the thick section 5515. For example, towels and sheets may be placed between the subject such as a patient and the radiographic imaging apparatus 100-14 in view of reducing burden on the subject such as a patient and maintaining hygiene etc. Here, even if the sheets or towels cover the entire radiographic imaging apparatus 100-14 including the thick section 5515, the user can easily figure out the contour of the effective imaging area by touching the outer shape of the thick section 5515. With conventional radiographic imaging apparatuses compliant with ISO 4090:2001, the outer shape of the housing is difficult to make identical to the effective imaging area, since the radiation detection panel is covered with the housing. By contrast, in the present exemplary embodiment, the housing 5007 includes the thin section 5008 and the thick section 5515, and the outer shape of the thick section 5515 in the width direction can be located on the extensions of the contour lines lying in the width direction of the effective imaging area. Configuring the thick section 5515 itself as a recognition portion thus facilities the user to recognize the contour of the effective imaging area compared to when steps are formed or the surface properties are changed.

The housing 5007 is also provided with a recognition portion 5520. The recognition portion 5520 according to the present exemplary embodiment is disposed on the top surface 5017 of the thick section 5515, on the extension of the center line 5509b of the indicator 5009. The recognition portion 5520 according to the present exemplary embodiment is implemented by a step. Specifically, the recognition portion 5520 has a protruding shape protruding from the top surface 5017. The recognition portion 5520 has a straight shape along the extension of the center line 5009b of the indicator 5009.

According to the present exemplary embodiment, the outer shape of the thick section 5515 in the width direction is located on the extensions of the contour lines lying in the width direction of the effective imaging area, and the thick section 5515 itself is configured as a recognition portion. This can facilitate the positioning of the effective imaging area.

### Fifteenth Exemplary Embodiment

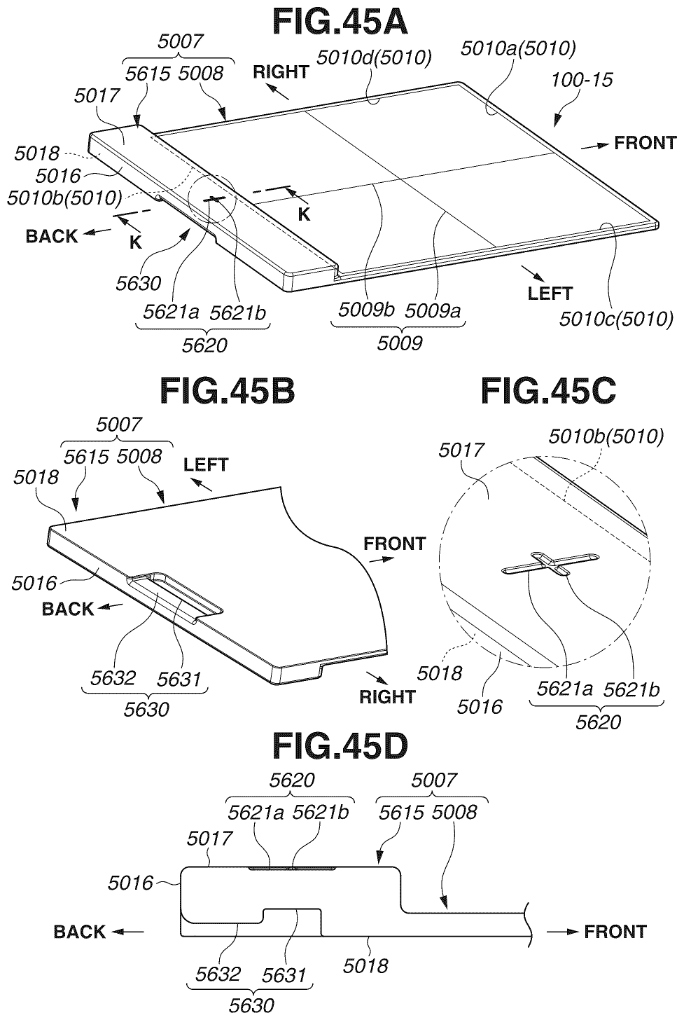

FIGS. 45A to 45D are diagrams illustrating an example of the appearance of a radiographic imaging apparatus 100-15 according to a fifteenth exemplary embodiment. Specifically, FIG. 45A is a perspective view illustrating a configuration of the radiographic imaging apparatus 100-15. FIG. 45B is a perspective view illustrating a part of the configuration of the radiographic imaging apparatus 100-15 as seen from the opposite side of FIG. 45A. FIG. 45C is an enlarged view of a recognition portion 5620. FIG. 45D is a sectional view taken along line K-K illustrated in FIG. 45A. Components similar to those of the twelfth exemplary embodiment are denoted by the same reference numerals, and a description thereof will be omitted. A housing 5007 according to the present exemplary embodiment includes a thin section 5008 and a thick section 5615. The thick section 5615 according to the present exemplary embodiment differs from the thick section 5015 according to the twelfth exemplary embodiment in configuration.

The thick section 5615 according to the present exemplary embodiment includes a grip portion 5630 for gripping the radiographic imaging apparatus 100-15. The grip portion 5630 according to the present exemplary embodiment is located on a bottom surface 5018 side of the thick section 5615, in the center of the thick section 5615 in the lateral direction (width direction). The grip portion 5630 includes a recess 5631 that is recessed from the bottom surface 5018, and a hand access portion 5632 that is cut in the side surface 5016 on the back side and communicates with the recess 5631. The recess 5631 is the part where fingertips are placed when the grip portion 5630 is gripped by hand. The hand access portion 5632 is the part where the hand (fingers) is placed when the grip portion 5630 is gripped by hand. Note that the grip portion 5630 according to the present exemplary embodiment is shaped so that the recess 5631 does not run through the top surface 5017, so that a volume capable of accommodating a control substrate 5005 and a battery 5006 is ensured within the thick section 5615.

The hand access portion 5632 can improve the user's accessibility to the grip portion 5630. Specifically, the user can insert the hand from the side surface 5016 on the back side through the hand access portion 5632 if the incident surface 5012 and the bottom surface 5013 of the thin section 5008 are covered, or even if the top surface 5017 and the bottom surface 5018 of the thick section 5615 are covered. The user can access the grip portion 5630 by inserting the hand through the hand access portion 5632, and can thus easily manipulate the radiographic imaging apparatus 100-15.

The housing 5007 according to the present exemplary embodiment includes the recognition portion 5620 disposed in the top surface 5017 of the thick section 5615. The recognition portion 5620 is implemented by a step. Specifically, the recognition portion 5620 is disposed in the top surface 5017 of the thick section 5615, on the extension of the center line 5009b of the indicator 5009. The recognition portion 5620 includes two straight line portions 5621a and 5621b. In a plan view, the straight line portions 5621a and 5621b intersect at right angles. The recognition portion 5620 is formed in a cross shape with the straight line portions 5621a and 5621b.

The straight line portion 5621a is located on the extension of the center line 5009b of the indicator 5009 and has a straight shape along the extension of the center line 5009b. The straight line portion 5621a is parallel to the contour lines 5010c and 5010d of the indicator 5010. The straight line portion 5621b is located on the extension of the center line 5009b of the indicator 5009 and has a straight shape orthogonal to the center line 5009b. In other words, the straight line portion 5621b is parallel to the center line 5009a. The straight line portion 5621b is also parallel to the contour lines 5010a and 5010b of the indicator 5010.

The recognition portion 5620 according to the present exemplary embodiment is disposed in the top surface 5017 opposite to the bottom surface 5018 where the grip portion 5630 is formed. As illustrated in FIG. 45D, the recognition portion 5620 and the recess 5631 of the grip portion 5630 are opposed to each other. When the user grips the grip portion 5630, the user places their fingers (thumb) on the top surface 5017 at the position opposed to the recess 5631 for a stable grip. Since the recognition portion 5620 is located at the position opposed to the grip portion 5630, the user can touch the recognition portion 5620 simultaneously with gripping the grip portion 5630, and can thus easily position the effective imaging area while manipulating the radiographic imaging apparatus 100-15. The present exemplary embodiment like this can eliminate the need to perform a touching operation on the recognition portion 5620 aside from the gripping operation, and the radiographic imaging apparatus 100-15 can be efficiently manipulated.

In the present exemplary embodiment, the grip portion 5630 is described to be located on the bottom surface 5018 side of the thick section 5615. However, the grip portion 5630 may be located on the top surface 5017 side opposite to the bottom surface 5018. If the grip portion 5630 is located on the top surface 5017 side, the recognition portion 5620 is desirably disposed in the bottom surface 5018 opposite to the top surface 5017 where the grip portion 5630 is formed. In the present exemplary embodiment, the recess 5631 of the grip portion 5630 may be configured as a through hole that runs through from the bottom surface 5018 to the top surface 5017. In such a case, the recognition portion 5620 can be disposed in the inner peripheral surface of the through hole of the grip portion 5630. In the present exemplary embodiment, the grip portion 5630 is described to include the hand access portion 5632. However, this is not restrictive, and the grip portion 5630 may be configured without the hand access portion 5632.

### Modification 1 of Fifth Exemplary Embodiment

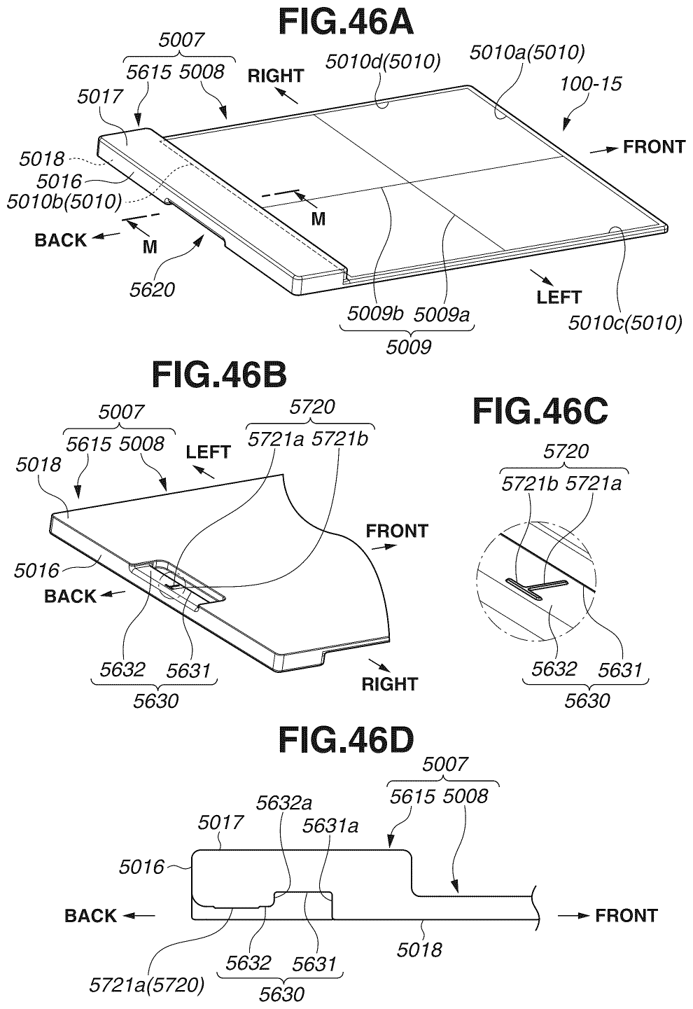

FIGS. 46A to 46D are diagrams illustrating modification 1 of the configuration of the radiographic imaging apparatus 100-15 according to the fifteenth exemplary embodiment. Specifically, FIG. 46A is a perspective view illustrating the configuration of the radiographic imaging apparatus 100-15 according to modification 1. FIG. 46B is a perspective view illustrating a part of the configuration of the radiographic imaging apparatus 100-15 as seen from the opposite side of FIG. 46A. FIG. 46C is an enlarged view of a recognition portion 5720. FIG. 46D is a sectional view taken along line M-M illustrated in FIG. 46A. In FIGS. 46A to 46D, components similar to those of FIGS. 45A to 45D are denoted by the same reference numerals, and a description thereof will be omitted.

The housing 5007 according to this modification includes the recognition portion 5720 disposed in the grip portion 5630 of the thick section 5615. The recognition portion 5720 is provided in the bottom surface of the hand access portion 5632 of the grip portion 5630. The recognition portion 5720 is implemented by a step. Specifically, the recognition portion 5720 is located on the extension of the center line 5009b of the indicator 5009. The recognition portion 5720 includes two straight line portions 5721a and 5721b. In a plan view, the straight line portions 5721a and 5721b intersect at right angles. The recognition portion 5720 is formed in a T shape with the straight line portions 5721a and 5721b.

Since the hand access portion 5632 of the grip portion 5630 is provided with the recognition portion 5720, the user can touch the recognition portion 5720 simultaneously with gripping the grip portion 5630, and can thus easily position the effective imaging area while manipulating the radiographic imaging apparatus 100-15. Such a modification eliminates the need to perform a touching operation on the recognition portion 5720 aside from the gripping operation, and the radiographic imaging apparatus 100-15 can be efficiently manipulated.

In the present exemplary embodiment, the recognition portion 5720 is described to be disposed in the bottom surface of the hand access portion 5632. However, the recognition portion 5720 may be disposed in a side surface 5632a of the hand access portion 5632 (see FIG. 46D), the bottom surface of the recess 5631, or a side surface 5631a of the recess 5631 (see FIG. 46D).

### Sixteenth Exemplary Embodiment

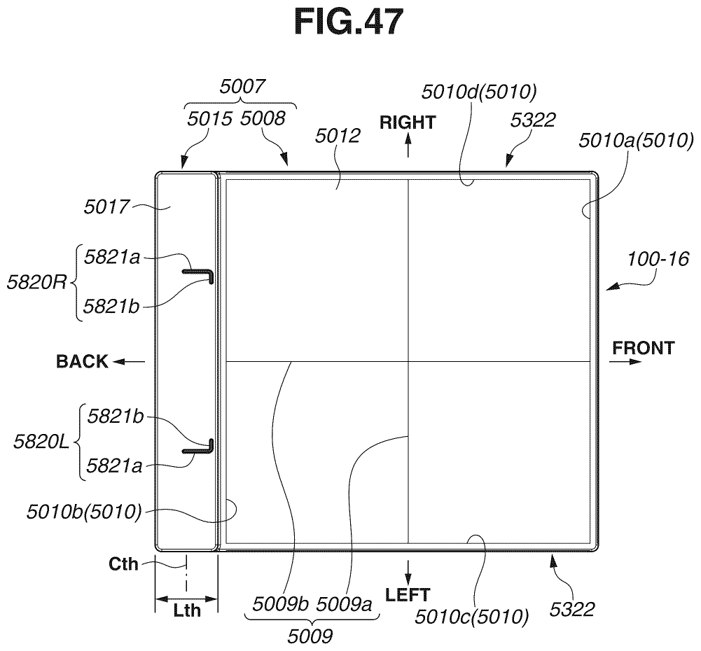

FIG. 47 is a diagram illustrating an example of the appearance of a radiographic imaging apparatus 100-16 according to a sixteenth exemplary embodiment. Specifically, FIG. 47 is a diagram illustrating a configuration of the radiographic imaging apparatus 100-16. Components similar to those of the foregoing twelfth to fifteenth exemplary embodiments are denoted by the same reference numerals, and a description thereof will be omitted. A housing 5007 according to the present exemplary embodiment includes a plurality of (here, two) recognition portions 5820L and 5820R on the top surface 5071 of the thick section 5015. The recognition portions 5820L and 5820R enable recognition of the vicinity of the center of the effective imaging area. The recognition portions 5820L and 5820R are located laterally apart from each other and laterally symmetrical. Here, the recognition portion 5820L will mainly be described. The recognition portion 5820L is formed in an L shape with a straight line portion 5821a and a straight line portion 5821b.

Here, the straight line portion 5821a is located midway between the extension of the center line 5009b of the indicator 5009 and the extension of the contour line 5010c of the indicator 5010. The recognition portions 5820L and 5820R thus enable the recognition of the vicinity of the center of the effective imaging area. The straight line portion 5821b functions similarly to the straight line portion 5421b illustrated in FIG. 43. The provision of the recognition portions 5820L and 5820R for enabling the recognition of the vicinity of the center of the effective imaging area on the thick section 5015 can prevent the recognition portions 5820L and 5820R from being overlooked compared to when a single recognition portion is provided or when the recognition portions are provided at the ends in the width direction.

While the preferred twelfth to sixteenth exemplary embodiments of the present invention have been described above, these exemplary embodiments are not restrictive, and various modifications and changes can be made without departing from the gist thereof. Moreover, the foregoing exemplary embodiments may be combined as appropriate. For example, the recognition portions described in the foregoing twelfth to sixteenth exemplary embodiments (including modifications) are described to enable the recognition of the center position of the effective imaging area, the contour of the effective imaging area, or the vicinity of the center of the effective imaging area. However, this is not restrictive. For example, the recognition portions may be intended to enable recognition of a given position of the effective imaging area.

In the foregoing twelfth to sixteenth exemplary embodiments (including modifications), the incident surface 5012 of the thin section 5008 is described to be provided with the indicator 5009 for indicating the center position of the effective imaging area and the indicator 5010 for indicating the contour of the effective imaging area. However, the indicator 5009 or the indicator 5010 may be omitted. For example, without the indicator 5009, the recognition portions 5020a, 5020b, 5120a, 5120b, 5220, 5320 (5321a), 5520, 5620 (5621a), and 5720 (5721a) can be located on the thick section, on the extension from the center position of the effective imaging area along the front-to-back direction. For example, without the indicator 5010, the recognition portions 5420R and 5420L (5421a) can be located on the thick section, on the extensions of the contour of the effective imaging area along the front-to-back direction. For example, without the indicator 5010, the left side surface 5016L of the thick section 5515 can be located on the extension of the contour (left end) of the effective imaging area along the front-to-back direction, and the right side surface 5016R of the thick section 5515 can be located on the extension of the contour (right end) of the effective imaging area along the front-to-back direction.

The foregoing twelfth to sixteenth exemplary embodiments (including modifications) can be combined with a part of another exemplary embodiment or a part of another modification, or replaced with a part of another exemplary embodiment or a part of another modification. For example, the recognition portion(s) of one exemplary embodiment may be applied to another exemplary embodiment or another modification. The recognition portion(s) of one modification may be applied to another exemplary embodiment or another modification.

The twelfth to sixteenth exemplary embodiments of the present exemplary embodiment include the features described in the following supplementary notes.

### [Supplementary Note 66]

A radiographic imaging apparatus including:
a radiation detection panel that includes an effective imaging area configured to detect radiation transmitted through a subject, the subject being irradiated with radiation from a radiation generation apparatus; and
a housing that accommodates the radiation detection panel,
wherein the housing includes
a thin section that overlaps the effective imaging area in a radiation incident direction, and
a thick section that is thicker than the thin section along the radiation incident direction, and
wherein the thick section includes a recognition portion configured to enable recognition of the effective imaging area.

### [Supplementary Note 67]

The radiographic imaging apparatus according to supplementary note 66, wherein the recognition portion indicates at least either of a center position of the effective imaging area and a contour of the effective imaging area.

### [Supplementary Note 68]

The radiographic imaging apparatus according to supplementary note 66 or 67,
wherein the thick section includes a top surface, a side surface, and a bottom surface, and
wherein the recognition portion is disposed on at least one of the top surface, the side surface, the bottom surface, a sloped surface between the top surface and the side surface, and a sloped surface between the bottom surface and the side surface.

### [Supplementary Note 69]

The radiographic imaging apparatus according to any one of supplementary notes 66 to 68,
wherein the thin section is provided with a center line indicating a/the center position of the effective imaging area, and
wherein the recognition portion is located on an extension of the center line. [Supplementary Note 70]

The radiographic imaging apparatus according to supplementary note 69, wherein the recognition portion includes a straight line portion along the extension of the center line.

### [Supplementary Note 71]

The radiographic imaging apparatus according to supplementary note 69 or 70,
wherein the center line is two orthogonal straight lines, and
wherein the recognition portion includes a first straight line portion parallel to one of the two straight lines and a second straight line portion parallel to the other of the two straight lines.

### [Supplementary Note 72]

The radiographic imaging apparatus according to any one of supplementary notes 66 to 68,
wherein the thin section is provided with a contour line indicating a/the contour of the effective imaging area, and
wherein the recognition portion is located on an extension of the contour line.

### [Supplementary Note 73]

The radiographic imaging apparatus according to supplementary note 72, wherein the recognition portion includes a straight line portion along the extension of the contour line.

### [Supplementary Note 74]

The radiographic imaging apparatus according to supplementary note 72 or 73,
wherein the contour line is at least two orthogonal straight lines, and
wherein the recognition portion includes a first straight line portion parallel to one of the two straight lines and a second straight line portion parallel to the other of the two straight lines.

### [Supplementary Note 75]

The radiographic imaging apparatus according to supplementary note 71 or 74, wherein the second straight line portion is located on a/the top surface of the thick section, at a position offset toward the thin section.

### [Supplementary Note 76]

The radiographic imaging apparatus according to any one of supplementary notes 66 to 75, wherein the recognition portion is implemented by locating an outer shape of the thick section in a width direction on an extension of a/the contour line of the effective imaging area located in the width direction.

### [Supplementary Note 77]

The radiographic imaging apparatus according to any one of supplementary notes 66 to 76,
wherein the thick section includes a/the top surface, a/the bottom surface, and a/the side surface,
wherein a grip portion for gripping the radiographic imaging apparatus is formed in the top surface or the bottom surface, and
wherein the recognition portion is disposed on one of the top surface and the bottom surface opposite to the surface where the grip portion is formed.

### [Supplementary Note 78]

The radiographic imaging apparatus according to any one of supplementary notes 66 to 76,
wherein the thick section includes a grip portion for gripping the radiographic imaging apparatus, and
wherein the recognition portion is disposed on the grip portion.

### [Supplementary Note 79]

The radiographic imaging apparatus according to supplementary note 78,
wherein the thick section includes a/the top surface, a/the bottom surface, and a/the side surface,
wherein the grip portion includes a hand access portion where a hand is placed when gripping the grip portion from the side of the side surface, and
wherein the recognition portion is disposed on the hand access portion.

### [Supplementary Note 80]

The radiographic imaging apparatus according to any one of supplementary notes 66 to 79, wherein the recognition portion is implemented by a step or by changing a surface property.

### [Supplementary Note 81]

The radiographic imaging apparatus according to any one of supplementary notes 66 to 79, wherein the recognition portion is implemented using a light source.

### [Supplementary Note 82]

The radiographic imaging apparatus according to any one of supplementary notes 66 to 79, wherein the recognition portion is implemented by applying color different from that of the thick section around the recognition portion.

According to the features described in the foregoing supplementary notes 66 to 82, the effective imaging area can be easily positioned.

### Seventeenth Exemplary Embodiment

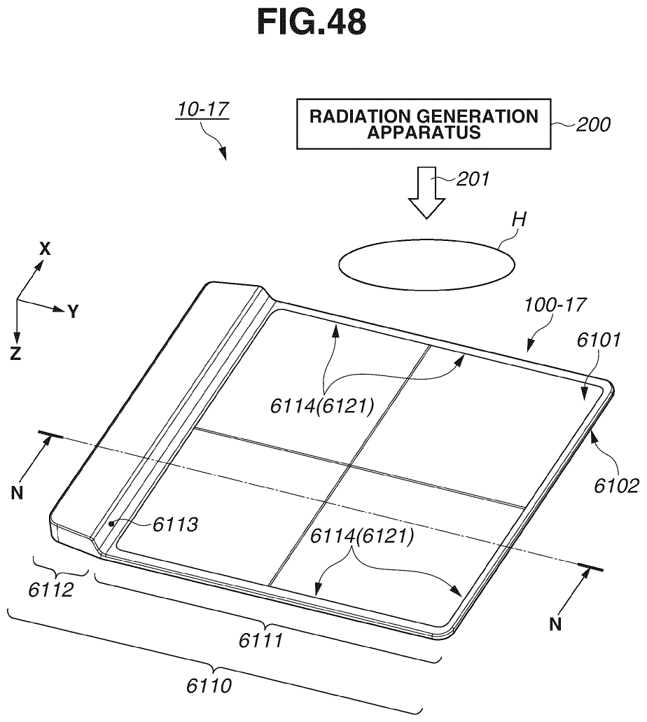

FIG. 48 is a diagram illustrating an example of a schematic configuration of a radiographic imaging system 10-17 according to a seventeenth exemplary embodiment. As illustrated in FIG. 48, the radiographic imaging system 10-17 includes a radiographic imaging apparatus 100-17 and a radiation generation apparatus 200.

The radiation generation apparatus 200 is an apparatus that emits radiation toward a subject H and the radiographic imaging apparatus 100-17.

The radiographic imaging apparatus 100-17 is an apparatus that detects incident radiation 201 (including the radiation 201 transmitted through the subject H) and obtains a radiographic image of the subject H. The radiographic image obtained by this radiographic imaging apparatus 100-17 is transferred to an external apparatus, displayed on a monitor by the external apparatus, and used for diagnosis and the like, for example. FIG. 48 illustrates a radiation incident surface 6101 that is the side where the radiation 201 is incident on the radiographic imaging apparatus 100-17, and a rear surface 6102 that is located opposite to the radiation incident surface 6101 (located at a position opposed to the radiation incident surface 6101). FIG. 48 also illustrates an XYZ coordinate system with the incident direction (vertical direction) of the radiation 201 as a Z direction and two mutually orthogonal directions orthogonal to the Z direction as an X direction and a Y direction. In the present exemplary embodiment, the Z direction of the XYZ coordinate system illustrated in FIG. 48 is the incident direction (vertical direction) of the foregoing radiation 201 and corresponds to a direction normal to the radiation incident surface 6101.

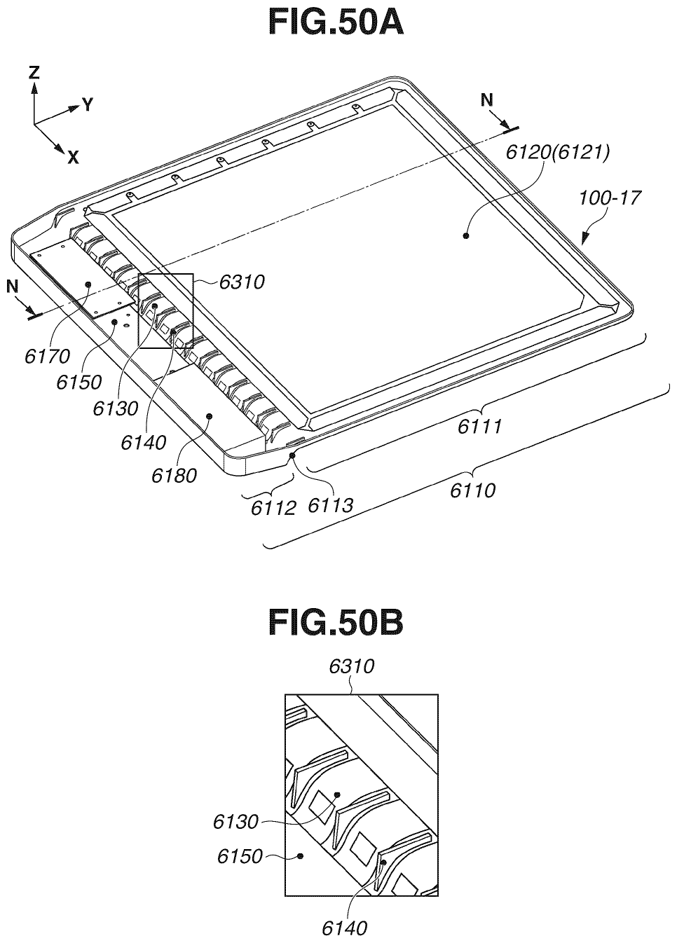

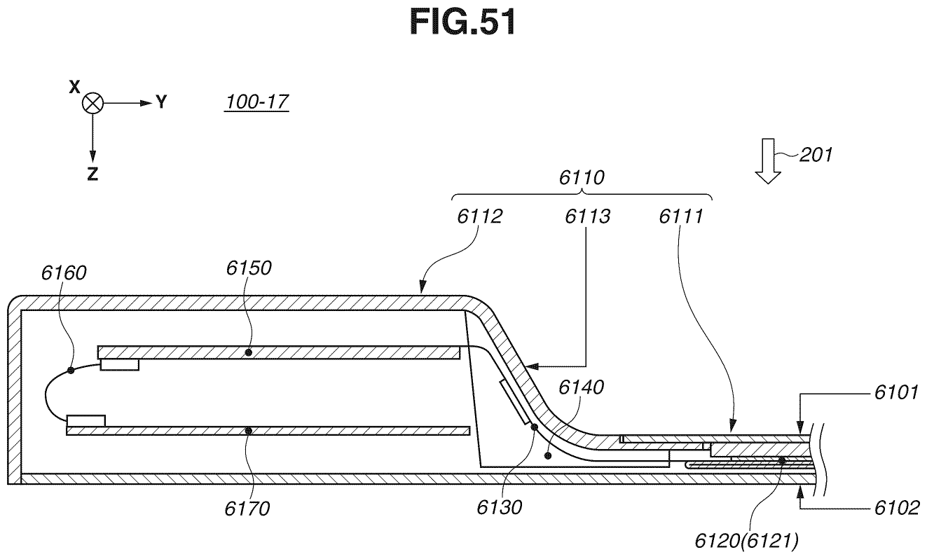

FIG. 48 illustrates a housing 6110 of the radiographic imaging apparatus 100-17 as the appearance of the radiographic imaging apparatus 100-17. An indicator 6114 indicating the range (including the center) of an effective imaging area 6121 where a radiation detection panel (radiation detection panel 6120 of FIGS. 50A, 50B, and 51 to be described below) accommodated inside this housing 6110 detects the radiation 201 transmitted through the subject H is displayed on the housing 6110.

As illustrated in FIG. 48, the housing 6110 includes a thin section 6111 corresponding to a first thickness section that is a section including the effective imaging area 6121 as seen in the Z direction, or the direction normal to the radiation incident surface 6101, and has a first thickness in the Z direction. As illustrated in FIG. 48, the housing 6110 also includes a thick section 6112 corresponding to a second thickness section that is a section not including the effective imaging area 6121 as seen in the Z direction, or the direction normal to the radiation incident surface 6101, and has a second thickness greater than the first thickness of the thin section 6111 in the Z direction. More specifically, in the example illustrated in FIG. 48, the thick section (second thickness section) 6112 is thicker than the thin section (first thickness section) 6111 at the side where the radiation 201 is incident. As illustrated in FIG. 48, the housing 6110 further includes a thickness changing section 6113 that connects the thick section (first thickness section) 6111 and the thick section (second thickness section) 6112 with a gradient.

The housing 6110 is a single- or multi-part housing including the thin section 6111, the thick section 6112, and the thickness changing section 6113 described above. The housing 6110 illustrated in FIG. 48 will now be described in more detail.

To achieve portability and strength in a compatible manner, the housing 6110 is desirably formed of a material such as a magnesium alloy, an aluminum alloy, and fiber-reinforced plastic, for example. However, in the present exemplary embodiment, the housing 6110 may be formed of materials other than those mentioned here. In particular, the radiation incident surface 6101 of the thin section 6111 where the effective imaging area 6121 is located is desirably formed of a carbon fiber-reinforced plastic with high transmittance for the radiation 201 and excellent lightweight properties, whereas other materials may also be used. When imaging the subject H such as a patient using the radiation 201, the radiographic imaging apparatus 100-17 can be placed immediately behind the imaging site of the object H. In doing so, due to a step created by the thickness of the housing 6110 of the radiographic imaging apparatus 100-17, the subject H and the end portion of the housing 6110 contact to cause reaction force, and the subject H such as a patient may feel discomfort. Typical radiographic imaging apparatuses are often provided in sizes compliant with International Organization for Standardization (ISO) 4090:2001, often with a thickness of approximately 15 mm to 16 mm. By contrast, in the radiographic imaging apparatus 100-17 according to the present exemplary embodiment, the thin section 6111 of the housing 6110 is assumed to have a thickness (first thickness) of 8.0 mm. With the radiographic imaging apparatus 100-17 according to the present exemplary embodiment, the step created by the thickness of the housing 6110 during radiographic imaging is thus smaller, and the reaction force occurring between the subject H and the end portion (thin section 6111) of the housing 6110 can be alleviated. To obtain such effects, the thickness of the thin section 6111 of the housing 6110 does not need to be limited to 8.0 mm, and may be even smaller, for example. The applicant has confirmed that the foregoing effects are obtained if the thickness of the housing 6110 is less than 10.0 mm. In the present exemplary embodiment, the thickness of the thin section 6111 of the foregoing housing 6110 is set to 8.0 mm as an appropriate thickness in view of the configuration and mechanical strength of the radiation detection panel (radiation detection panel 6120 in FIGS. 50A, 50B, and 51 to be described below) disposed in the thin section 6111.

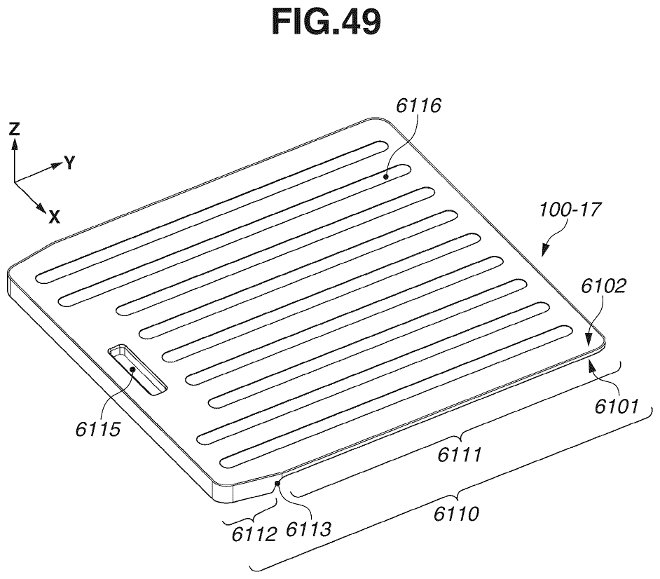

FIG. 49 is a view of the radiographic imaging apparatus 100-17 according to the seventeenth exemplary embodiment, seen from the rear surface 6102 side. In this FIG. 49, components similar to those illustrated in FIG. 48 are denoted by the same reference numerals, and a detailed description thereof will be omitted. FIG. 49 also illustrates an XYZ coordinate system corresponding to the XYZ coordinate system illustrated in FIG. 48.

As illustrated in FIG. 49, the radiographic imaging apparatus 100-17 includes a grip portion 6115 for the user to grip the housing 6110 in the rear surface 6102 of the thick section (second thickness section) 6112 of the housing 6110.

As illustrated in FIG. 49, the rear surface 6102 of the housing 6110 is provided with recessed reinforcement portions 6116 for supplementing bending rigidity of the housing 6110, whereby damage (including deformation and fracture) due to mechanical stress on the housing 6110 can be prevented. The recessed reinforcement portions 6116 can be disposed to extend from the thin section 6111 to the thick section 6112 with the thickness changing section 6113 of the housing 6110 therebetween as seen in the Z direction that is the direction normal to the radiation incident surface 6101. This can prevent the concentration of mechanical stress on the thickness changing section 6113, the thin section 6111, or the like, for example. If a part of the housing 6110 is formed of a carbon fiber-reinforced plastic, the housing 6110 can be designed to improve the strength in the Y direction of FIG. 49.

FIGS. 50A and 50B are diagrams illustrating an example of an internal configuration of the radiographic imaging apparatus 100-17 according to the seventeenth exemplary embodiment as seen from the rear surface 6102 side. In FIGS. 50A and 50B, components similar to those illustrated in FIGS. 48 and 49 are denoted by the same reference numerals, and a detailed description thereof will be omitted. FIGS. 50A and 50B also illustrate an XYZ coordinate system corresponding to the XYZ coordinate system illustrated in FIGS. 48 and 49. Specifically, FIG. 50A is a diagram illustrating the example of the internal configuration of the radiographic imaging apparatus 100-17 as seen from the rear surface 6102 side. FIG. 50B is an enlarged view of an area 6310 in FIG. 50A.

As illustrated in FIG. 50A, the radiographic imaging apparatus 100-17 includes the radiation detection panel 6120, flexible circuit boards 6130, protruding reinforcement portions 6140, a control substrate 6150, a processing substrate 6170, and a battery 6180 in its housing 6110. As illustrated in FIG. 50A, the control substrate 6150, the processing substrate 6170, and the battery 6180 are disposed in the thick section 6112.

The radiation detection panel 6120 has the effective imaging area 6121 illustrated in FIG. 48, which detects the radiation 201 emitted from the radiation generation apparatus 200 and incident thereon (including the radiation 201 transmitted through the subject H). In the present exemplary embodiment, the effective imaging area 6121 is substantially the same as the rectangular area of the radiation detection panel 6120 on the XY plane illustrated in FIG. 50A, whereas the effective imaging area 6121 may be an area inside and narrower than the rectangular area of the radiation detection panel 6120. The radiation detection panel 6120 can be implemented by a so-called indirect conversion system, including a sensor substrate on which a large number of photoelectric conversion elements (sensors) are arranged, a phosphor layer (scintillator layer) that is located above the sensor substrate, and a phosphor protective layer, for example. Here, the sensor substrate can be formed of a material such as glass and flexible plastic, but the present exemplary embodiment is not limited thereto. The phosphor protective layer is formed of a material with low moisture permeability and used to protect the phosphor layer. In the radiation detection panel 6120 of this indirect conversion system, the incident radiation 201 is converted into light by the phosphor layer, and the light obtained in the phosphor layer is converted into electrical signals by the photoelectric conversion elements, whereby image signals related to a radiographic image are generated. In the present exemplary embodiment, the radiation detection panel 6120 includes all the photoelectric conversion elements (sensors) in its effective imaging area 6121, whereas the effective imaging area 6121 may be constituted by some of the photoelectric conversion elements (sensors). The effective imaging area 6121 is an area that is capable of radiographic imaging of the subject H and where the radiographic image is actually generated. As illustrated in FIGS. 48 and 50A, the effective imaging area 6121 of the radiation detection panel 6120 is located in the thin section 6111. In the example illustrated in FIG. 48, the effective imaging area 6121 has a substantially rectangular shape as seen in the Z direction that is the incident direction of the radiation 201. However, the present exemplary embodiment is not limited to this substantially rectangular shape. The radiation detection panel 6120 is not limited to the configuration of the indirect conversion system, either, and may be configured using a so-called direct conversion system, including a conversion element unit where conversion elements formed of a-Se or the like and switch elements such as TFTs are two-dimensionally arranged. In this radiation detection panel 6120 of the direct conversion system, the incident radiation 201 is converted into electrical signals by the respective conversion elements, whereby image signals related to a radiographic image are generated.

The flexible circuit boards 6130 are a plurality of boards that includes various substrates and elements inside and connects the radiation detection panel 6120 and the control substrate 6150.

The protruding reinforcement portions 6140 are disposed in contact with the thickness changing section 6113 in at least a part of the thickness changing section 6113. The provision of the protruding reinforcement portions 6140 can reduce the possibility of damage (including deformation and fracture) of the radiographic imaging apparatus 100-17 even when mechanical stress concentrates on the thickness changing section 6113 located at the border between the thin section 6111 and the thick section 6112 of the housing 6110. In the radiographic imaging apparatus 100-17 according to the present exemplary embodiment, as illustrated in FIGS. 50A and 50B, the plurality of protruding reinforcement portions 6140 is located at positions not overlapping the flexible circuit boards 6130 as seen in the Z direction that is the direction normal to the radiation incident surface 6101. In other words, the plurality of protruding reinforcement portions 6140 is disposed between the flexible circuit boards 6130. Here, each protruding reinforcement portion 6140 in the plurality of protruding reinforcement portions 6140 is formed with a thickness width (length in the X direction) equivalent to or less than a basic thickness of the housing 6110, for example.

The control substrate 6150 is a substrate that controls driving of the radiation detection panel 6120 via the flexible circuit boards 6130. Moreover, the control substrate 6150 obtains the image signals related to the radiographic image from the radiation detection panel 6120 via the flexible circuit boards 6130.

The processing substrate 6170 is a substrate that processes the image signals related to the radiographic image, which are the signals output from the radiation detection panel 6120. Specifically, the processing substrate 6170 obtains the image signals related to the radiographic image output from the radiation detection panel 6120 via the control substrate 6150, and processes the obtained image signals related to the radiographic image.

The battery 6180 is a power supply that supplies power to the components of the radiographic imaging apparatus 100-17 (such as the radiation detection panel 6120, the flexible circuit boards 6130, the control substrate 6150, and the processing substrate 6170). Examples of the battery 6180 include a lithium-ion battery, an electric double layer capacitor, and an all-solid-state battery, whereas other batteries may be used. As illustrated in FIG. 50A, the battery 6180 is located in an area where the processing substrate 6170 is not disposed in the thick section 6112 of the housing 6110 as seen in the Z direction that is the incident direction of the radiation 201.

As illustrated in FIG. 50A, the control substrate 6150 and the processing substrate 6170 are located to overlap at least in part in the thick section 6112 of the housing 6110 as seen in the Z direction that is the incident direction of the radiation 201. By thus locating the control substrate 6150 and the processing substrate 6170 in the thick section 6112 of the housing 6110 to overlap as seen in the incident direction of the radiation 201 (Z direction), the area of the thick section 6112 in the planar direction (XY-plane direction) can be reduced.

Moreover, as illustrated in FIG. 50A, the control substrate 6150 and the battery 6180 are located to overlap at least in part in the thick section 6112 of the housing 6110 as seen in the Z direction that is the incident direction of the radiation 201. By thus locating the control substrate 6105 and the battery 6180 in the thick section 6112 of the housing 6110 to overlap as seen in the incident direction of the radiation 201 (Z direction), the area of the thick section 6112 in the planar direction (XY-plane direction) can be reduced.

FIG. 51 is a sectional view illustrating an example of the internal configuration of the radiographic imaging apparatus 100-17 according to the seventeenth exemplary embodiment illustrated in FIGS. 48 and 50A, taken along line N-N. In this FIG. 51, components similar to those illustrated in FIGS. 48 to 50B are denoted by the same reference numerals, and a detailed description thereof will be omitted. FIG. 51 also illustrates an XYZ coordinate system corresponding to the XYZ coordinate system illustrated in FIGS. 48 to 50B. Specifically, the cross section taken along line N-N illustrated in FIGS. 48 and 50A is a cross section along the Y direction.

As illustrated in FIG. 51, the housing 6110 of the radiographic imaging apparatus 100-17 includes the radiation detection panel 6120, the flexible circuit boards 6130, the protruding reinforcement portions 6140, the control substrate 6150, wiring 6160, and the processing substrate 6170.

The protruding reinforcement portions 6140 are reinforcement portions of protruding shape that are disposed in contact with the thickness changing section 6113 in at least a part of the thickness changing section 6113 of the housing 6110 and protrude in the Z direction that is the direction normal to the radiation incident surface 6101. Specifically, in the example illustrated in FIG. 51, the protruding reinforcement portions 6140 are located at least at the border between the thickness changing section 6113 and the thin section 6111 of the housing 6110. In the example illustrated in FIG. 51, the protruding reinforcement portions 6140 are in contact with the thickness changing section 6113 from inside the housing 6110. In the example illustrated in FIG. 51, the protruding reinforcement portions 6140 are disposed up to a part of the thick section 6112 of the housing 6110. In this regard, the protruding reinforcement portions 6104 may be extended throughout the thick section 6112 of the housing 6110 (up to the left end of the thick section 6112 illustrated in FIG. 51). Here, the configuration where the protruding reinforcement portions 6140 are disposed up to at least a part of the thick section 6112 of the housing 6110 is employed. The present exemplary embodiment is not limited to such a configuration, and may include a configuration where the protruding reinforcement portions 6140 are not disposed in the area of the thick section 6112 of the housing 6110.

As illustrated in FIG. 51, the control substrate 6150 is located in the thick section 6112 of the housing 6110, on the incident side of the radiation 201 relative to the processing substrate 6170. In other words, in the example illustrated in FIG. 51, the control substrate 6150 and the processing substrate 6170 are arranged in this order as seen from the radiation incident surface 6101 side of the thick section 6112.

The wiring 6160 is wiring that connects the control substrate 6150 and the processing substrate 6170. As illustrated in FIG. 51, this wiring 6160 is located on the side opposite to where the radiation detection panel 6120 is disposed relative to the control substrate 6150 and the processing substrate 6170.

As illustrated in FIG. 51, the radiation detection panel 6120 and the control substrate 6150 are located at different positions (heights) in the Z direction that is the incident direction of the radiation 201 (direction normal to the radiation incident surface 6101). The fixable circuit boards 6130 connect the radiation detection panel 6120 and the control substrate 6150 with a gradient with respect to the Y direction that is a horizontal direction. As illustrated in FIG. 51, the flexible circuit boards 6130 are disposed at least in part in the thickness changing section 6113 of the housing 6110. The flexible circuit boards 6130 include various substrates and elements inside, and therefore need a predetermined area. For example, if the flexible circuit boards 6130 are located in parallel with the Y direction perpendicular to the incident direction (Z direction) of the radiation 201, the radiographic imaging apparatus 100-17 increases in size in the planar direction (plane including the Y direction). In the present exemplary embodiment, the flexible circuit boards 6130 can be situated with a gradient, which can reduce the area of the flexible circuit boards 6130 in the planar direction (plane including the Y direction). As illustrated in FIG. 51, situating the flexible circuit boards 6130 with a gradient can thus achieve space saving in the planar direction in the radiographic imaging apparatus 100-17 (for example, the thick section 6112), and an increase in size can be prevented.

The thickness changing section 6113 of the housing 6110 has a gradient. In the radiographic imaging apparatus 100-17 according to the present exemplary embodiment, the gradient of the thickness changing section 6113 is configured to follow that of the flexible circuit boards 6103. However, the gradients do not necessarily need to be the same.

In the example illustrated in FIG. 51, the rigid member of the thin section 6111 of the housing 6110, such as magnesium alloy, and the radiation incident surface 6101 formed of a member with excellent transmittance for the radiation 201, such as carbon fiber-reinforced plastic, are bonded at the joint surface. The joint surface of the rigid member, such as magnesium alloy, of the thin section 6111 is therefore locally made thinner.

In the example illustrated in FIG. 51, the protruding reinforcement portions 6140 are formed in the area including the thickness changing section 6113 and a part of the joint surface of the thin section 6111 of the housing 6110. However, the protruding reinforcement portions 6140 may be extended up to the outer shape portion of the housing 6110. In the radiographic imaging apparatus 100-17 according to the present exemplary embodiment, as illustrated in FIGS. 50A and 50B, the protruding reinforcement portions 6140 are disposed in the spaces between the flexible circuit boards 6130, whereby a sufficient height can be provided in the incident direction of the radiation 201 (Z direction). While FIG. 51 illustrates an example where there is a gap between the protruding reinforcement portions 6140 and the rear surface 6102 of the housing 6110, the protruding reinforcement portions 6140 and the rear surface 6102 of the housing 6110 may contact each other.

According to the seventeenth exemplary embodiment, by providing the protruding reinforcement portions 6140 in contact with the thickness changing section 6113 rather than increasing the basic thickness of the entire thickness changing section 6113, damage due to the concentration of mechanical stress on the thickness changing section 6113 can be prevented without providing external protrusions and recesses. In particular, according to the seventeenth exemplary embodiment, damage due to the concentration of mechanical stress on the border between the thickness changing section 6113 and the thin section 6111 can be prevented. This can implement a structure that ensures the rigidity of the radiographic imaging apparatus 100-17 while suppressing an increase in its weight (a structure with the thin section 6111 and the thickness changing section 6113 of reduced thickness).

### Eighteenth Exemplary Embodiment

Next, an eighteenth exemplary embodiment will be described. In the following description of the eighteenth exemplary embodiment, a description of items common to the foregoing seventeenth exemplary embodiment are omitted, and differences from the foregoing seventeenth exemplary embodiment will be described.

A schematic configuration of a radiographic imaging system according to the eighteenth exemplary embodiment is similar to that of the radiographic imaging system 10-17 according to the seventeenth exemplary embodiment illustrated in FIG. 48.

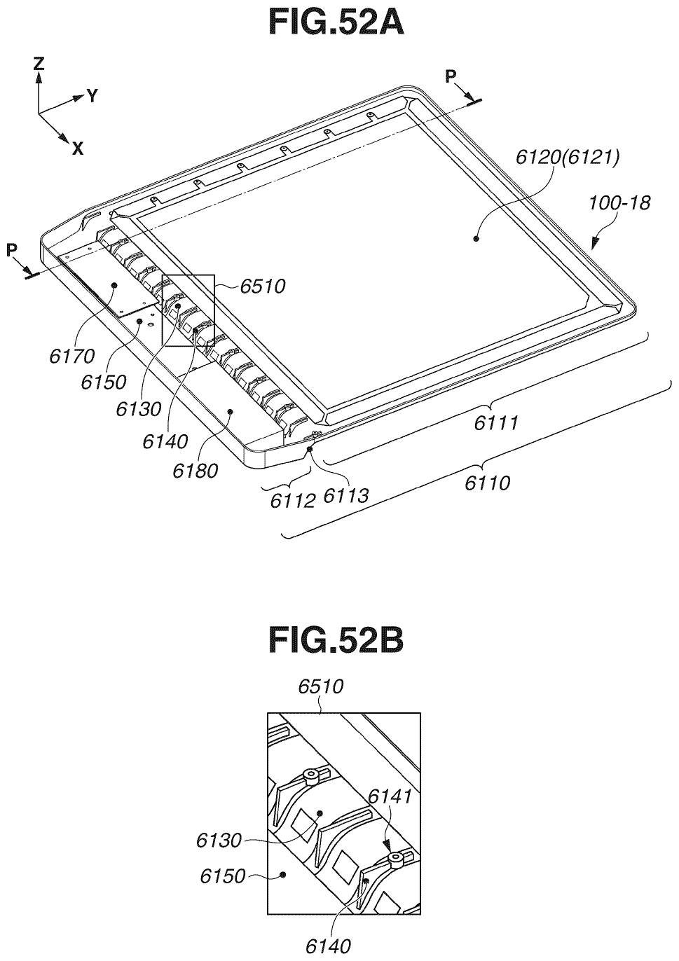

FIGS. 52A and 52B are diagrams illustrating an example of the internal configuration of a radiographic imaging apparatus 100-18 according to the eighteenth exemplary embodiment as seen from a rear surface 6102 side. In FIGS. 52A and 52B, components similar to those illustrated in FIGS. 48 to 51 are denoted by the same reference numerals, and a detailed description thereof will be omitted. FIGS. 52A and 52B illustrate an XYZ corresponding to the XYZ coordinate system illustrated in FIGS. 48 to 51. Specifically, FIG. 52A is a diagram illustrating an example of the internal configuration of the radiographic imaging apparatus 100-18 as seen from the rear surface 6102 side. FIG. 52B is an enlarged view of an area 6510 in FIG. 52A.

As illustrated in FIG. 52A, the radiographic imaging apparatus 100-18 includes a radiation detection panel 6120, flexible circuit boards 6130, protruding reinforcement portions 6140, a control substrate 6150, a processing substrate 6170, and a battery 6180 in its housing 6110. As illustrated in FIG. 52A, the control substrate 6150, the processing substrate 6170, and the battery 6180 are located in a thick section 6112.

In the radiographic imaging apparatus 100-18 according to the present exemplary embodiment, as illustrated in FIGS. 52A and 52B, a plurality of protruding reinforcement portions 6140 is located at positions not overlapping the flexible circuit boards 6130 as seen in a Z direction that is a direction orthogonal to the radiation incident surface 6101. More specifically, the plurality of protruding reinforcement portions 6140 is disposed between the flexible circuit boards 6103. Even in the radiographic imaging apparatus 100-18 according to the present exemplary embodiment, as illustrated in FIGS. 52A and 52B, the protruding reinforcement portions 6140 are disposed in the spaces between the flexible circuit boards 6130, whereby a sufficient height can be provided in the incident direction of radiation 201 (Z direction). In the radiographic imaging apparatus 100-18 according to the present exemplary embodiment, as illustrated in FIG. 52B, at least one protruding reinforcement portion 6140 in the plurality of protruding reinforcement portions 6140 is provided with a pillar portion 6141.

As illustrated in FIG. 52A, even with the radiographic imaging apparatus 100-18 according to the present exemplary embodiment, the control substrate 6150 and the processing substrate 6170 are located in the thick section 6112 of the housing 6110 to overlap at least in part when seen in the Z direction that is the incident direction of the radiation 201. Moreover, as illustrated in FIG. 52A, the control substrate 6150 and the battery 6180 are located to overlap at least in part in the thick section 6112 of the housing 6110 when seen in the Z direction that is the incident direction of the radiation 201. Furthermore, as illustrated in FIG. 52A, the battery 6180 is located in an area where the processing substrate 6170 is not located in the thick section 6112 of the housing 6110 when seen in the Z direction that is the incident direction of the radiation 201.

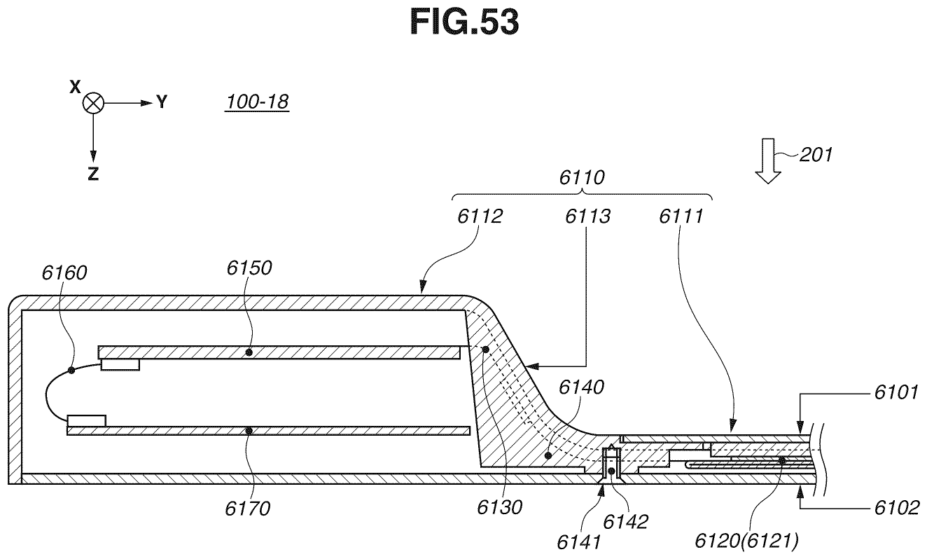

FIG. 53 is a sectional view illustrating an example of the internal configuration of the radiographic imaging apparatus 100-18 according to the eighteenth exemplary embodiment illustrated in FIGS. 52A and 52B, taken along line P-P. In this FIG. 53, components similar to those illustrated in FIGS. 48 to 52B are denoted by the same reference numerals, and a detailed description thereof will be omitted. FIG. 53 also illustrates an XYZ coordinate system corresponding to the XYZ coordinate system illustrated in FIGS. 52A and 52B. Specifically, the cross section taken along line P-P illustrated in FIGS. 52A and 52B is a cross section along the Y direction.

As illustrated in FIG. 53, the pillar portion 6141 provided on at least one protruding reinforcement portion 6140 in the plurality of protruding reinforcement portions 6140 is configured to be in contact with the rear surface 6102 side of the housing 6110. In the example illustrated in FIG. 53, the pillar portion 6141 is configured as a cylindrical pillar portion. However, in the present exemplary embodiment, the shape of the pillar portion 6141 is not limited to the cylindrical shape illustrated in FIG. 53. The pillar portion 6141 provided on at least one protruding reinforcement portion 6140 in the plurality of protruding reinforcement portions 6140 has a threaded hole. This at least one protruding reinforcement portion 6140 is then fixed to the rear surface 6102 of the housing 6110 by a fixing member 6142, such as a screw, via the threaded hole formed in the pillar portion 6141. In other words, the protruding reinforcement portion 6140 illustrated in FIG. 53 is in contact with and fixed to at least a part of the inner side of the rear surface 6102 opposed to the radiation incident surface 6101 in the housing 6110. If the plurality of protruding reinforcement portions 6140 is provided with a plurality of fixing members 6142, the positions in the Y direction do not need to be aligned on the same straight line in the X direction, and the positions in the Y direction may be changed depending on the respective protruding reinforcement portions 6140. This can ease stress concentration on the thickness changing section 6113 located at the border between the thin section 6111 and the thick section 6112. Although not illustrated in the drawings, the protruding reinforcement portions 6140 may be extended from the rear surface 6102 side of the housing 6110.

According to the eighteenth exemplary embodiment, the rigidity of the thickness changing section 6113 located at the border between the thick section 6112 and the thin section 6111 of the housing 6110 can be increased, and damage (including deformation and fracture) due to the concentration of mechanical stress can be prevented.

### Nineteenth Exemplary Embodiment

Next, a nineteenth exemplary embodiment will be described. In the following description of the nineteenth exemplary embodiment, a description of items common to the foregoing seventeenth and eighteenth exemplary embodiments is omitted, and differences from the foregoing seventeenth and eighteenth exemplary embodiment will be described.

In the foregoing seventeenth and eighteenth exemplary embodiments, the protruding reinforcement portions 6140 are disposed in contact with the thickness changing section 6113 from inside the housing 6110. In the nineteenth exemplary embodiment, protruding reinforcement portions 6140 are disposed in contact with the thickness changing section 6113 from outside the housing 6110.

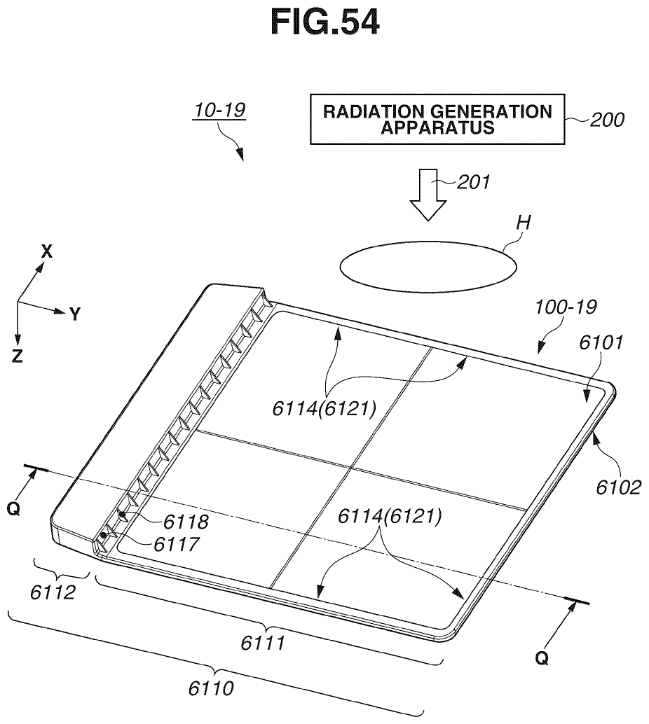

FIG. 54 is a diagram illustrating an example of a schematic configuration of a radiographic imaging system 10-19 according to the nineteenth exemplary embodiment. As illustrated in FIG. 54, the radiographic imaging system 10-19 includes a radiographic imaging apparatus 100-19 and a radiation generation apparatus 200. In this FIG. 54, components similar to those illustrated in FIG. 48 are denoted by the same reference numerals, and a detailed description thereof will be omitted. FIG. 54 also illustrates an XYZ coordinate system corresponding to the XYZ coordinate system illustrated in FIG. 48.

As illustrated in FIG. 54, the housing 6110 includes a thin section 6111 corresponding to a first thickness section that is a section including an effective imaging area 6121 as seen in a Z direction, or a direction normal to a radiation incident surface 6101, and has a first thickness in the Z direction. As illustrated in FIG. 54, the housing 6110 also includes a thick section 6112 corresponding to a second thickness section that is a section not including the effective imaging area 6121 as seen in the Z direction, or the direction normal to the radiation incident surface 6101, and has a second thickness greater than the first thickness of the thin section 6111 in the Z direction. As illustrated in FIG. 54, the housing 6110 further includes a thickness changing section 6117 that connects the thin section (first thickness section) 6111 and the thick section (second thickness section) 6112 with a gradient.

The radiographic imaging apparatus 100-19 according to the present exemplary embodiment includes a plurality of protruding reinforcement portions 6118 that is in contact with the thickness changing section 6117 from outside the housing 6110. Specifically, in the example illustrated in FIG. 54, a plurality of protruding reinforcement portions 6118 having a thickness width (length in the X direction) equivalent to or less than a basic thickness of the housing 6110, for example, is disposed in an area including the border between the thin section 6111 and the thickness changing section 6117 of the housing 6110. The provision of the protruding reinforcement portions 6118 can prevent damage (including deformation and fracture) due to the concentration of mechanical stress on the border between the thickness changing section 6117 and the thin section 6111 of the housing 6110. This can implement a structure that ensures the rigidity of the radiographic imaging apparatus 100-19 while suppressing an increase in its weight (a structure with the thin section 6111 and the thickness changing section 6117 of reduced thickness).

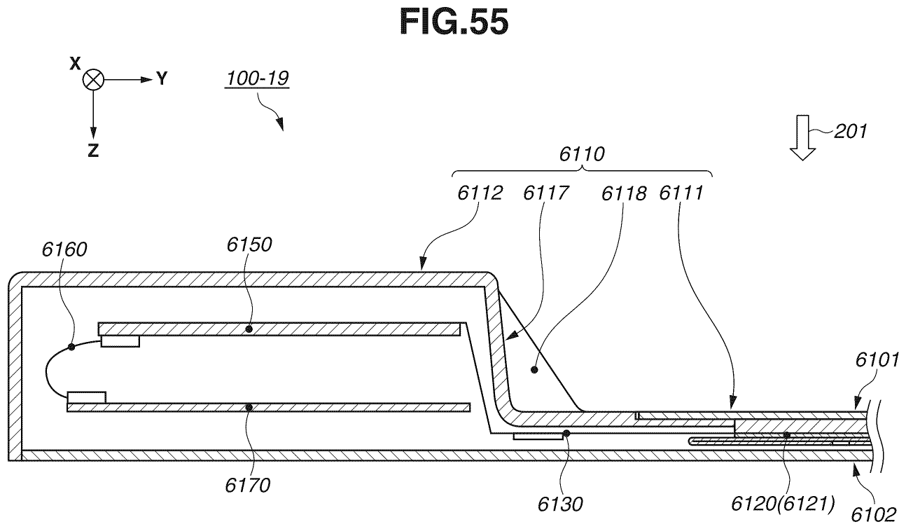

FIG. 55 is a sectional view illustrating an example of the internal configuration of the radiographic imaging apparatus 100-19 according to the nineteenth exemplary embodiment illustrated in FIG. 54, taken along line Q-Q. In this FIG. 55, components similar to those illustrated in FIGS. 48 to 54 are denoted by the same reference numerals, and a detailed description thereof will be omitted. FIG. 55 also illustrates an XYZ coordinate system corresponding to the XYZ coordinate system illustrated in FIG. 54. Specifically, the cross section taken along line Q-Q illustrated in FIG. 54 is a cross section along the Y direction.

As illustrated in FIG. 55, the radiation detection panel 6120 and the control substrate 6150 are located at different positions (heights) in the Z direction that is the incident direction of radiation 201. The flexible circuit boards 6130 thus connect the radiation detection panel 6120 and the control substrate 6150 with a gradient with respect to the Y direction that is a horizontal direction. As illustrated in FIG. 55, the flexible circuit boards 6130 are disposed at least in part in the thickness changing section 6117 of the housing 6110. The flexible circuit boards 6130 include various substrates and elements inside, and therefore need a predetermined area. The surfaces of the flexible circuit boards 6130 where the substrates and elements are disposed are therefore situated in parallel with the Y direction perpendicular to the incident direction of the radiation 201 (Z direction), for example. As the surfaces where the substrates and elements are disposed are situated in the XY-plane direction, the flexible circuit boards 6130 are connected to the control substrate 6150 with a gradient nearly parallel to the Z direction. This achieves space saving in the XY-plane direction in the radiographic imaging apparatus 100-19.

In the present exemplary embodiment, to reduce the thickness of the thin section 6111 of the housing 6110 in the Z direction, the protruding reinforcement portions 6118 are disposed outside the housing 6110, not inside the housing 6110.

According to the nineteenth exemplary embodiment, the rigidity of the thickness changing section 6117 located at the border between the thick section 6112 and the thin section 6111 of the housing 6110 can be increased without increasing the thickness of the thin section 6111 of the housing 6110 in the Z direction. This can prevent damage (including deformation and fracture) due to the concentration of mechanical stress on the thickness changing section 6117.

While the preferred seventeenth to nineteenth exemplary embodiments of the present invention have been described above, these exemplary embodiments are not restrictive, and various modifications and changes can be made without departing from the gist thereof. Moreover, the foregoing exemplary embodiments may be combined as appropriate.

The seventeenth to nineteenth exemplary embodiments of the present invention include the features described in the following supplementary notes.

### [Supplementary Note 83]

A radiographic imaging apparatus including:
a radiation detection panel that includes an effective imaging area configured to detect incident radiation; and
a housing that has an incident surface where the radiation is incident, and accommodates the radiation detection panel,
wherein the housing includes
a first thickness section that has a first thickness in a direction normal to the incident surface and includes the effective imaging area as seen in the normal direction,
a second thickness section that has a second thickness greater than the first thickness in the normal direction and does not include the effective imaging area as seen in the normal direction, and
a thickness changing section that connects the first thickness section and the second thickness section, and
wherein a protruding reinforcement portion that is disposed in contact with the thickness changing section and protrudes in the normal direction is included in at least a part of the thickness changing section.
   [Supplementary note 84]

The radiographic imaging apparatus according to supplementary note 83, wherein the protruding reinforcement portion is located at least at a border between the thickness changing section and the first thickness section.

### [Supplementary Note 85]

The radiographic imaging apparatus according to supplementary note 83 or 84, further including:
a control substrate configured to control driving of the radiation detection panel; and
a flexible circuit board configured to connect the radiation detection panel and the control substrate,
wherein at least a part of the flexible circuit board is disposed in the thickness changing section, and
wherein the protruding reinforcement portion is located at a position not overlapping the flexible circuit board as seen in the normal direction.

### [Supplementary Note 86]

The radiographic imaging apparatus according to any one of supplementary notes 83 to 85, wherein the protruding reinforcement portion is in contact with the thickness changing section from inside the housing.

### [Supplementary Note 87]

The radiographic imaging apparatus according to supplementary note 86, wherein the protruding reinforcement portion is in contact with at least a part of an inner side of a rear surface opposed to the incident surface in the housing.

### [Supplementary Note 88]

The radiographic imaging apparatus according to supplementary note 86, wherein the protruding reinforcement portion is in contact with and fixed to at least a part of an inner side of a rear surface opposed to the incident surface in the housing.

### [Supplementary Note 89]

The radiographic imaging apparatus according to any one of supplementary notes 83 to 85, wherein the protruding reinforcement portion is in contact with the thickness changing section from outside the housing.

### [Supplementary Note 90]

The radiographic imaging apparatus according to any one of supplementary notes 83 to 88, wherein the protruding reinforcement portion is disposed up to at least a part of the second thickness section.

### [Supplementary Note 91]

The radiographic imaging apparatus according to any one of supplementary notes 83 to 90, wherein a recessed reinforcement portion is disposed in a/the rear surface opposed to the incident surface in the housing.

### [Supplementary Note 92]

The radiographic imaging apparatus according to supplementary note 91, wherein the recessed reinforcement portion is disposed to extend from the first thickness section to the second thickness section as seen in the normal direction.

### [Supplementary Note 93]

A radiographic imaging system including:
the radiographic imaging apparatus according to any one of supplementary notes 83 to 92; and
a radiation generation apparatus configured to generate the radiation.

According to the features described in the foregoing supplementary notes 83 to 93, the possibility of damage of the radiographic imaging apparatus when stress is applied to the radiographic imaging apparatus can be reduced.

The present invention is not limited to the foregoing exemplary embodiments, and various modifications and changes can be made without departing from the spirit and scope of the present invention. The following claims are therefore attached to make public the scope of the present invention.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

1. A radiographic imaging apparatus comprising:
a radiation detection panel that includes an effective imaging area configured to detect radiation transmitted through a subject and incident on an incident surface; and
a housing that accommodates the radiation detection panel,
wherein the housing includes a thick section that is thick in a direction normal to the incident surface and located at one end of the housing and a thin section that is thinner than the thick section and overlaps the effective imaging area at least in part as seen in the direction normal to the incident surface, and
wherein the thick section includes a grip portion of recessed shape.

2. The radiographic imaging apparatus according to claim 1,
wherein the thick section includes a thick incident surface where the radiation is incident and a thick rear surface opposed to the thick incident surface, and
wherein the grip portion is disposed in at least either of the thick incident surface and the thick rear surface.

3. The radiographic imaging apparatus according to claim 2, wherein the grip portion includes an incident-side grip portion that is a grip portion disposed in the thick incident surface and a rear-side grip portion that is a grip portion disposed in the thick rear surface.

4. The radiographic imaging apparatus according to claim 3, wherein the incident-side grip portion has a length of 20 mm or more and the rear-side grip portion has a length of 60 mm or more in a direction along a border between the thin section and the thick section.

5. The radiographic imaging apparatus according to claim 3, wherein a depth of the rear-side grip portion from the thick rear surface is greater than a depth of the incident-side grip portion from the thick incident surface.

6. The radiographic imaging apparatus according to claim 3, wherein a sum of a depth of the incident-side grip portion from the thick incident surface and a depth of the rear-side grip portion from the thick rear surface is 5 mm or more.

7. The radiographic imaging apparatus according to claim 2,
wherein the thick section includes a thick side surface connecting the thick incident surface and the thick rear surface, and
wherein a hand access portion of recessed shape is disposed to adjoin the thick side surface and the thick rear surface.

8. The radiographic imaging apparatus according to claim 7, wherein the hand access portion includes a hand access surface adjoining the grip portion and the thick side surface.

9. The radiographic imaging apparatus according to claim 8, wherein a depth of the hand access surface from the thick rear surface is smaller than that of the grip portion.

10. The radiographic imaging apparatus according to claim 2, wherein the thick rear surface is inclined with respect to a surface of the thin section opposed to the incident surface.

11. The radiographic imaging apparatus according to claim 1,
wherein the thick section includes a control unit configured to control the radiation detection panel and a power supply unit configured to supply power to each component of the radiographic imaging apparatus, and
wherein the grip portion is located at a position overlapping at least either of the control unit and the power supply unit as seen in the direction normal to the incident surface.

12. A radiographic imaging apparatus comprising:
a radiation detection panel that includes an effective imaging area configured to detect radiation transmitted through a subject and incident on an incident surface; and
a housing that accommodates the radiation detection panel,
wherein the housing includes a thick section that is thick in a direction normal to the incident surface and located at one end of the housing, and a thin section that is thinner than the thick section and overlaps the effective imaging area at least in part as seen in the direction normal to the incident surface, and
wherein a sloped portion is disposed on at least a part of a side opposed to the thick section among a plurality of sides of the thin section, the sloped portion sloping at an end portion of the thin section.

13. A radiographic imaging apparatus comprising:
a radiation detection panel that includes an effective imaging area configured to detect radiation transmitted through a subject, the subject being irradiated with radiation from a radiation generation apparatus; and
a housing that accommodates the radiation detection panel,
wherein the housing includes
a thin section that overlaps the effective imaging area in a radiation incident direction,
a thick section that is thicker than the thin section along the radiation incident direction, and
a recognition portion that is provided in a border region between the thin section and the thick section and configured to enable recognition of a border between the thin section and the thick section, the recognition portion located in an area outside ends of the thick section in a longitudinal direction thereof.

14. A radiographic imaging apparatus comprising:
a housing including a front surface constituting an incident surface of radiation, a rear surface opposed to the front surface, and a side surface connecting the front surface and the rear surface; and
a radiation detection panel accommodated in the housing,
wherein a low-friction region with a coefficient of kinetic friction of 0.15 or less is disposed on at least either of the front surface and the rear surface of the housing.

15. A radiographic imaging apparatus comprising:
a radiation detection panel that includes an effective imaging area configured to detect radiation transmitted through a subject, the subject being irradiated with radiation from a radiation generation apparatus; and
a housing that accommodates the radiation detection panel,
wherein the housing includes
a thin section that overlaps the effective imaging area in a radiation incident direction, and
a thick section that is thicker than the thin section along the radiation incident direction, and
wherein the thick section includes a recognition portion configured to enable recognition of the effective imaging area.

16. A radiographic imaging apparatus comprising:
a radiation detection panel that includes an effective imaging area configured to detect incident radiation; and
a housing that has an incident surface where the radiation is incident, and accommodates the radiation detection panel,
wherein the housing includes
a first thickness section that has a first thickness in a direction normal to the incident surface and includes the effective imaging area as seen in the normal direction,
a second thickness section that has a second thickness greater than the first thickness in the normal direction and does not include the effective imaging area as seen in the normal direction, and
a thickness changing section that connects the first thickness section and the second thickness section, and
wherein a protruding reinforcement portion that is disposed in contact with the thickness changing section and protrudes in the normal direction is included in at least a part of the thickness changing section.
